SEQUENCE LISTING (944 sequences)

SEQ ID NO: 1 - protein, 18 aa, Artificial Sequence
  Homo sapiens Aggrecan core protein precursor epitope
AGMDMCSAGWLADRSVRY

SEQ ID NO: 2 - protein, 18 aa, Artificial Sequence
  Homo sapiens Aggrecan core protein precursor epitope
EDSEATLEVVVKGIVFHY

SEQ ID NO: 3 - protein, 18 aa, Artificial Sequence
  Homo sapiens Aggrecan core protein precursor epitope
SRVSKEKEVVLLVATEGR

SEQ ID NO: 4 - protein, 18 aa, Artificial Sequence
  Homo sapiens Aggrecan core protein precursor epitope
VVLLVATEGRVRVNSAYQ

SEQ ID NO: 5 - protein, 18 aa, Artificial Sequence
  Homo sapiens Aggrecan core protein precursor epitope
VVVKGIVFHYRAISTRYT

SEQ ID NO: 6 - protein, 9 aa, Artificial Sequence
  Homo sapiens alpha 2 type VI collagen isoform 2C2 precursor epitope
DRASFIKNL

SEQ ID NO: 7 - protein, 20 aa, Artificial Sequence
  Homo sapiens arrestin epitope
ASSTIIKEGIDRTVLGILVS

SEQ ID NO: 8 - protein, 20 aa, Artificial Sequence
  Homo sapiens arrestin epitope
ASTPTKLQESLLKKLGSNTY

SEQ ID NO: 9 - protein, 20 aa, Artificial Sequence
  Homo sapiens arrestin epitope
DRTVLGILVSYQIKVKLTVS

SEQ ID NO: 10 - protein, 20 aa, Artificial Sequence
  Homo sapiens arrestin epitope
EFARHNLKDAGEAEEGKRDK

SEQ ID NO: 11 - protein, 20 aa, Artificial Sequence
  Homo sapiens arrestin epitope
EPNHVIFKKISRDKSVTIYL

SEQ ID NO: 12 - protein, 20 aa, Artificial Sequence
  Homo sapiens arrestin epitope
FEVKAFATDSTDAEEDKIPK

SEQ ID NO: 13 - protein, 20 aa, Artificial Sequence
  Homo sapiens arrestin epitope
GFLGELTSSEVATEVPFRLM

SEQ ID NO: 14 - protein, 20 aa, Artificial Sequence
  Homo sapiens arrestin epitope
GKIKHEDTNLASSTIIKEGI

SEQ ID NO: 15 - protein, 20 aa, Artificial Sequence
  Homo sapiens arrestin epitope
GNRDYIDHVSQVQPVDGVVL

SEQ ID NO: 16 - protein, 20 aa, Artificial Sequence
  Homo sapiens arrestin epitope
KPVAMEEAQEKVPPNSTLTK

SEQ ID NO: 17 - protein, 20 aa, Artificial Sequence
  Homo sapiens arrestin epitope
KVPPNSTLTKTLTLLPLLAN

SEQ ID NO: 18 - protein, 20 aa, Artificial Sequence
  Homo sapiens arrestin epitope
LLKKLGSNTYPFLLTFPDYL

SEQ ID NO: 19 - protein, 20 aa, Artificial Sequence
  Homo sapiens arrestin epitope
LTFRRDLYFSRVQVYPPVGA

SEQ ID NO: 20 - protein, 20 aa, Artificial Sequence
  Homo sapiens arrestin epitope
MAASGKTSKSEPNHVIFKKI

SEQ ID NO: 21 - protein, 20 aa, Artificial Sequence
  Homo sapiens arrestin epitope
NRERRGIALDGKIKHEDTNL

SEQ ID NO: 22 - protein, 20 aa, Artificial Sequence
  Homo sapiens arrestin epitope
PCSVMLQPAPQDSGKSCGVD

SEQ ID NO: 23 - protein, 20 aa, Artificial Sequence
  Homo sapiens arrestin epitope
PFLLTFPDYLPCSVMLQPAP

SEQ ID NO: 24 - protein, 20 aa, Artificial Sequence
  Homo sapiens arrestin epitope
QDSGKSCGVDFEVKAFATDS

SEQ ID NO: 25 - protein, 20 aa, Artificial Sequence
  Homo sapiens arrestin epitope
QVQPVDGVVLVDPDLVKGKK

SEQ ID NO: 26 - protein, 20 aa, Artificial Sequence
  Homo sapiens arrestin epitope
RVQVYPPVGAASTPTKLQES

SEQ ID NO: 27 - protein, 20 aa, Artificial Sequence
  Homo sapiens arrestin epitope
SRDKSVTIYLGNRDYIDHVS

SEQ ID NO: 28 - protein, 20 aa, Artificial Sequence
  Homo sapiens arrestin epitope
TLTLLPLLANNRERRGIALD

SEQ ID NO: 29 - protein, 20 aa, Artificial Sequence
  Homo sapiens arrestin epitope
VATEVPFRLMHPQPEDPAKE

SEQ ID NO: 30 - protein, 20 aa, Artificial Sequence
  Homo sapiens arrestin epitope
VDPDLVKGKKVYVTLTCAFR

SEQ ID NO: 31 - protein, 20 aa, Artificial Sequence
  Homo sapiens arrestin epitope
VVLYSSDYYVKPVAMEEAQE

SEQ ID NO: 32 - protein, 20 aa, Artificial Sequence
  Homo sapiens arrestin epitope
YQIKVKLTVSGFLGELTSSE

SEQ ID NO: 33 - protein, 9 aa, Artificial Sequence
  Homo sapiens Chain B, Structure Of Insulin epitope
ALYLVCGER

SEQ ID NO: 34 - protein, 10 aa, Artificial Sequence
  Homo sapiens Chain B, Structure Of Insulin epitope
SHLVEALYLV

SEQ ID NO: 35 - protein, 9 aa, Artificial Sequence
  Homo sapiens chaperonin (HSP60) epitope
QMRPVSRVL

SEQ ID NO: 36 - protein, 9 aa, Artificial Sequence
  Homo sapiens Collagen alpha-3(IV) chain epitope
GSPATWTTR

SEQ ID NO: 37 - protein, 9 aa, Artificial Sequence
  Homo sapiens collagen, type II, alpha 1 isoform 1 precursor epitope
ARGQPGVMG

SEQ ID NO: 38 - protein, 9 aa, Artificial Sequence
  Homo sapiens DNA topoisomerase 1 epitope
KMLDHEYTT

SEQ ID NO: 39 - protein, 9 aa, Artificial Sequence
  Homo sapiens ezrin epitope
EYTAKIALL

SEQ ID NO: 40 - protein, 10 aa, Artificial Sequence
  Homo sapiens ezrin epitope
LNIYEKDDKL

SEQ ID NO: 41 - protein, 9 aa, Artificial Sequence
  Homo sapiens glial fibrillary acidic protein isoform 2 epitope
NLAQDLATV

SEQ ID NO: 42 - protein, 9 aa, Artificial Sequence
  Homo sapiens glial fibrillary acidic protein isoform 2 epitope
QLARQQVHV

SEQ ID NO: 43 - protein, 9 aa, Artificial Sequence
  Homo sapiens glucagon receptor epitope
RRRWHRWRL

SEQ ID NO: 44 - protein, 9 aa, Artificial Sequence
  Homo sapiens glucose-6-phosphatase, catalytic, related epitope
FLWSVFWLI

SEQ ID NO: 45 - protein, 15 aa, Artificial Sequence
  Homo sapiens Glutamate decarboxylase 1 epitope
NMFTYEIAPVFVLME

SEQ ID NO: 46 - protein, 13 aa, Artificial Sequence
  Homo sapiens Glutamate decarboxylase 2 epitope
IAFTSEHSHFSLK

SEQ ID NO: 47 - protein, 13 aa, Artificial Sequence
  Homo sapiens Glutamate decarboxylase 2 epitope
NFFRMVISNPAAT

SEQ ID NO: 48 - protein, 9 aa, Artificial Sequence
  Homo sapiens Glutamate decarboxylase 2 epitope
FLQDVMNIL

SEQ ID NO: 49 - protein, 9 aa, Artificial Sequence
  Homo sapiens Glutamate decarboxylase 2 epitope
LLQEYNWEL

SEQ ID NO: 50 - protein, 10 aa, Artificial Sequence
  Homo sapiens Glutamate decarboxylase 2 epitope
RMMEYGTTMV

SEQ ID NO: 51 - protein, 10 aa, Artificial Sequence
  Homo sapiens Glutamate decarboxylase 2 epitope
VMNILLQYVV

SEQ ID NO: 52 - protein, 11 aa, Artificial Sequence
  Homo sapiens Glutamate decarboxylase 2 epitope
AFTSEHSHFSL

SEQ ID NO: 53 - protein, 12 aa, Artificial Sequence
  Homo sapiens Glutamate decarboxylase 2 epitope
AFTSEHSHFSLK

SEQ ID NO: 54 - protein, 11 aa, Artificial Sequence
  Homo sapiens Glutamate decarboxylase 2 epitope
FKMFPEVKEKG

SEQ ID NO: 55 - protein, 10 aa, Artificial Sequence
  Homo sapiens Glutamate decarboxylase 2 epitope
FTSEHSHFSL

SEQ ID NO: 56 - protein, 15 aa, Artificial Sequence
  Homo sapiens Glutamate decarboxylase 2 epitope
MIARFKMFPEVKEKG

SEQ ID NO: 57 - protein, 9 aa, Artificial Sequence
  Homo sapiens Glutamate decarboxylase 2 epitope
RFKMFPEVK

SEQ ID NO: 58 - protein, 10 aa, Artificial Sequence
  Homo sapiens Glutamate decarboxylase 2 epitope
RFKMFPEVKE

SEQ ID NO: 59 - protein, 11 aa, Artificial Sequence
  Homo sapiens Glutamate decarboxylase 2 epitope
RFKMFPEVKEK

SEQ ID NO: 60 - protein, 9 aa, Artificial Sequence
  Homo sapiens Glutamate decarboxylase 2 epitope
TSEHSHFSL

SEQ ID NO: 61 - protein, 9 aa, Artificial Sequence
  Homo sapiens Glutamate decarboxylase 2 epitope
VMNILLQYV

SEQ ID NO: 62 - protein, 9 aa, Artificial Sequence
  Homo sapiens Glutamate decarboxylase 2 epitope
ELAEYLYNI

SEQ ID NO: 63 - protein, 9 aa, Artificial Sequence
  Homo sapiens Glutamate decarboxylase 2 epitope
ILMHCQTTL

SEQ ID NO: 64 - protein, 11 aa, Artificial Sequence
  Homo sapiens heat shock 27kDa protein 1 epitope
QLSSGVSEIRH

SEQ ID NO: 65 - protein, 9 aa, Artificial Sequence
  Homo sapiens HLA class I histocompatibility antigen, B-27 alpha chain precursor epitope
LRRYLENGK

SEQ ID NO: 66 - protein, 9 aa, Artificial Sequence
  Homo sapiens HLA class I histocompatibility antigen, B-7 alpha chain precursor epitope
VMAPRTVLL

SEQ ID NO: 67 - protein, 14 aa, Artificial Sequence
  Homo sapiens HLA class I histocompatibility antigen, B-7 alpha chain precursor epitope
ALNEDLRSWTAADT

SEQ ID NO: 68 - protein, 14 aa, Artificial Sequence
  Homo sapiens HLA-B27 epitope
ALNEDLSSWTAADT

SEQ ID NO: 69 - protein, 10 aa, Artificial Sequence
  Homo sapiens HLA-B27 epitope
LLRGYHQDAY

SEQ ID NO: 70 - protein, 15 aa, Artificial Sequence
  Homo sapiens HLA-B27 epitope
RVAEQLRAYLEGECV

SEQ ID NO: 71 - protein, 13 aa, Artificial Sequence
  Homo sapiens HLA-B27 epitope
WDRETQICKAKAQ

SEQ ID NO: 72 - protein, 11 aa, Artificial Sequence
  Homo sapiens insulin epitope
ALWGPDPAAAF

SEQ ID NO: 73 - protein, 10 aa, Artificial Sequence
  Homo sapiens insulin epitope
LALWGPDPAA

SEQ ID NO: 74 - protein, 11 aa, Artificial Sequence
  Homo sapiens insulin epitope
RLLPLLALLAL

SEQ ID NO: 75 - protein, 9 aa, Artificial Sequence
  Homo sapiens Insulin precursor epitope
ALWMRLLPL

SEQ ID NO: 76 - protein, 9 aa, Artificial Sequence
  Homo sapiens Insulin precursor epitope
HLVEALYLV

SEQ ID NO: 77 - protein, 10 aa, Artificial Sequence
  Homo sapiens Insulin precursor epitope
SLQKRGIVEQ

SEQ ID NO: 78 - protein, 9 aa, Artificial Sequence
  Homo sapiens Insulin precursor epitope
SLQPLALEG

SEQ ID NO: 79 - protein, 9 aa, Artificial Sequence
  Homo sapiens Insulin precursor epitope
SLYQLENYC

SEQ ID NO: 80 - protein, 10 aa, Artificial Sequence
  Homo sapiens Insulin precursor epitope
VCGERGFFYT

SEQ ID NO: 81 - protein, 8 aa, Artificial Sequence
  Homo sapiens Insulin precursor epitope
WGPDPAAA

SEQ ID NO: 82 - protein, 9 aa, Artificial Sequence
  Homo sapiens Insulin precursor epitope
FYTPKTRRE

SEQ ID NO: 83 - protein, 8 aa, Artificial Sequence
  Homo sapiens Insulin precursor epitope
GERGFFYT

SEQ ID NO: 84 - protein, 9 aa, Artificial Sequence
  Homo sapiens Insulin precursor epitope
ERGFFYTPK

SEQ ID NO: 85 - protein, 10 aa, Artificial Sequence
  Homo sapiens Insulin precursor epitope
LCGSHLVEAL

SEQ ID NO: 86 - protein, 10 aa, Artificial Sequence
  Homo sapiens Insulin precursor epitope
LVCGERGFFY

SEQ ID NO: 87 - protein, 10 aa, Artificial Sequence
  Homo sapiens Insulin precursor epitope
LYLVCGERGF

SEQ ID NO: 88 - protein, 9 aa, Artificial Sequence
  Homo sapiens Islet amyloid polypeptide precursor epitope
FLIVLSVAL

SEQ ID NO: 89 - protein, 9 aa, Artificial Sequence
  Homo sapiens Islet amyloid polypeptide precursor epitope
KLQVFLIVL

SEQ ID NO: 90 - protein, 9 aa, Artificial Sequence
  Homo sapiens islet-specific glucose-6-phosphatase-related protein epitope
FLWSVFMLI

SEQ ID NO: 91 - protein, 9 aa, Artificial Sequence
  Homo sapiens islet-specific glucose-6-phosphatase-related protein isoform 1 epitope
FLFAVGFYL

SEQ ID NO: 92 - protein, 9 aa, Artificial Sequence
  Homo sapiens islet-specific glucose-6-phosphatase-related protein isoform 1 epitope
LNIDLLWSV

SEQ ID NO: 93 - protein, 9 aa, Artificial Sequence
  Homo sapiens islet-specific glucose-6-phosphatase-related protein isoform 1 epitope
VLFGLGFAI

SEQ ID NO: 94 - protein, 9 aa, Artificial Sequence
  Homo sapiens islet-specific glucose-6-phosphatase-related protein isoform 1 epitope
NLFLFLFAV

SEQ ID NO: 95 - protein, 9 aa, Artificial Sequence
  Homo sapiens islet-specific glucose-6-phosphatase-related protein isoform 1 epitope
YLLLRVLNI

SEQ ID NO: 96 - protein, 9 aa, Artificial Sequence
  Homo sapiens keratin 6C epitope
ALQKAKQDL

SEQ ID NO: 97 - protein, 9 aa, Artificial Sequence
  Homo sapiens keratin 6C epitope
DAKNKLEGL

SEQ ID NO: 98 - protein, 9 aa, Artificial Sequence
  Homo sapiens keratin 6C epitope
GASGVGSGL

SEQ ID NO: 99 - protein, 9 aa, Artificial Sequence
  Homo sapiens keratin 6C epitope
KAKQDLARL

SEQ ID NO: 100 - protein, 9 aa, Artificial Sequence
  Homo sapiens keratin 6C epitope
KLEGLEDAL

SEQ ID NO: 101 - protein, 9 aa, Artificial Sequence
  Homo sapiens keratin 6C epitope
NMQDLVEDL

SEQ ID NO: 102 - protein, 9 aa, Artificial Sequence
  Homo sapiens keratin 6C epitope
RLLKEYQEL

SEQ ID NO: 103 - protein, 9 aa, Artificial Sequence
  Homo sapiens keratin 6C epitope
WYQTKYEEL

SEQ ID NO: 104 - protein, 20 aa, Artificial Sequence
  Homo sapiens Keratin, type I cytoskeletal 17 (Cytokeratin 17) (K17) (CK 17) (Version 2) epitope
LRRVLDELTLARTDLEMQIE

SEQ ID NO: 105 - protein, 9 aa, Artificial Sequence
  Homo sapiens Keratin, type I cytoskeletal 17 (Cytokeratin 17) (K17) (CK 17) (Version 2) epitope
ALEEANADL

SEQ ID NO: 106 - protein, 9 aa, Artificial Sequence
  Homo sapiens Keratin, type I cytoskeletal 17 (Cytokeratin 17) (K17) (CK 17) (Version 2) epitope
ANADLEVKI

SEQ ID NO: 107 - protein, 9 aa, Artificial Sequence
  Homo sapiens Keratin, type I cytoskeletal 17 (Cytokeratin 17) (K17) (CK 17) (Version 2) epitope
ARTDLEMQI

SEQ ID NO: 108 - protein, 9 aa, Artificial Sequence
  Homo sapiens Keratin, type I cytoskeletal 17 (Cytokeratin 17) (K17) (CK 17) (Version 2) epitope
ASYLDKVRA

SEQ ID NO: 109 - protein, 9 aa, Artificial Sequence
  Homo sapiens Keratin, type I cytoskeletal 17 (Cytokeratin 17) (K17) (CK 17) (Version 2) epitope
DVNGLRRVL

SEQ ID NO: 110 - protein, 9 aa, Artificial Sequence
  Homo sapiens Keratin, type I cytoskeletal 17 (Cytokeratin 17) (K17) (CK 17) (Version 2) epitope
GLRRVLDEL

SEQ ID NO: 111 - protein, 12 aa, Artificial Sequence
  Homo sapiens Keratin, type I cytoskeletal 17 (Cytokeratin 17) (K17) (CK 17) (Version 2) epitope
ISSVLAGASCPA

SEQ ID NO: 112 - protein, 9 aa, Artificial Sequence
  Homo sapiens Keratin, type I cytoskeletal 17 (Cytokeratin 17) (K17) (CK 17) (Version 2) epitope
LDKVRALEE

SEQ ID NO: 113 - protein, 9 aa, Artificial Sequence
  Homo sapiens Keratin, type I cytoskeletal 17 (Cytokeratin 17) (K17) (CK 17) (Version 2) epitope
QIEGLKEEL

SEQ ID NO: 114 - protein, 12 aa, Artificial Sequence
  Homo sapiens Keratin, type I cytoskeletal 17 (Cytokeratin 17) (K17) (CK 17) (Version 2) epitope
RALEEANADLEV

SEQ ID NO: 115 - protein, 9 aa, Artificial Sequence
  Homo sapiens Keratin, type I cytoskeletal 17 (Cytokeratin 17) (K17) (CK 17) (Version 2) epitope
RLASYLDKV

SEQ ID NO: 116 - protein, 8 aa, Artificial Sequence
  Homo sapiens Keratin, type I cytoskeletal 17 (Cytokeratin 17) (K17) (CK 17) (Version 2) epitope
SYLDKVRA

SEQ ID NO: 117 - protein, 20 aa, Artificial Sequence
  Homo sapiens Keratin, type I cytoskeletal 17 (Cytokeratin 17) (K17) (CK 17) (Version 2) epitope
SYLDKVRALEEANADLEVKI

SEQ ID NO: 118 - protein, 9 aa, Artificial Sequence
  Homo sapiens maspin epitope
GLEKIEKQL

SEQ ID NO: 119 - protein, 10 aa, Artificial Sequence
  Homo sapiens maspin epitope
MGNIDSINCK

SEQ ID NO: 120 - protein, 9 aa, Artificial Sequence
  Homo sapiens maspin epitope
YSLKLIKRL

SEQ ID NO: 121 - protein, 20 aa, Artificial Sequence
  Homo sapiens MBP protein epitope
ASQKRPSQRHGSKYLATAST

SEQ ID NO: 122 - protein, 15 aa, Artificial Sequence
  Homo sapiens MBP protein epitope
ENPVVHFFKNIVTPR

SEQ ID NO: 123 - protein, 9 aa, Artificial Sequence
  Homo sapiens MBP protein epitope
VVHFFKNIV

SEQ ID NO: 124 - protein, 19 aa, Artificial Sequence
  Homo sapiens MBP protein epitope
DENPVVHFFKNIVTPRTPP

SEQ ID NO: 125 - protein, 20 aa, Artificial Sequence
  Homo sapiens MBP protein epitope
HHPARTAHYGSLPQKSHGRT

SEQ ID NO: 126 - protein, 20 aa, Artificial Sequence
  Homo sapiens MBP protein epitope
VVHFFKNIVTPRTPPPSQGK

SEQ ID NO: 127 - protein, 21 aa, Artificial Sequence
  Homo sapiens MBP protein epitope
ASQKRPSQRHGSKYLATASTM

SEQ ID NO: 128 - protein, 20 aa, Artificial Sequence
  Homo sapiens MBP protein epitope
FKGVDAQGTLSKIFKLGGRD

SEQ ID NO: 129 - protein, 19 aa, Artificial Sequence
  Homo sapiens MBP protein epitope
RPGFGYGGRASDYKSAHKG

SEQ ID NO: 130 - protein, 38 aa, Artificial Sequence
  Homo sapiens MBP protein epitope
ASQKRPSQRHGSKYLATASTMDHARHGFLPRHRDTGIL

SEQ ID NO: 131 - protein, 9 aa, Artificial Sequence
  Homo sapiens MBP protein epitope
KYLATASTM

SEQ ID NO: 132 - protein, 20 aa, Artificial Sequence
  Homo sapiens MBP protein epitope
GLSLSRFSWGAEGQRPGFGY

SEQ ID NO: 133 - protein, 43 aa, Artificial Sequence
  Homo sapiens MBP protein epitope
FGGDRGAPKRGSGKDSHHPARTAHYGSLPQKSHGRTQDENPVV

SEQ ID NO: 134 - protein, 40 aa, Artificial Sequence
  Homo sapiens MBP protein epitope
GLSLSRFSWGAEGQRPGFGYGGRASDYKSAHKGFKGVDAQ

SEQ ID NO: 135 - protein, 9 aa, Artificial Sequence
  Homo sapiens MHC class I related protein A epitope
AAAAAIFVI

SEQ ID NO: 136 - protein, 9 aa, Artificial Sequence
  Homo sapiens Myelin basic protein epitope
SLSRFSWGA

SEQ ID NO: 137 - protein, 9 aa, Artificial Sequence
  Homo sapiens Myelin basic protein epitope
DYKSAHKGF

SEQ ID NO: 138 - protein, 19 aa, Artificial Sequence
  Homo sapiens myelin basic protein epitope
SKIFKLGGRDSRSGSPMAR

SEQ ID NO: 139 - protein, 8 aa, Artificial Sequence
  Homo sapiens myelin basic protein epitope
TPRTPPPQ

SEQ ID NO: 140 - protein, 9 aa, Artificial Sequence
  Homo sapiens myelin proteolipid protein epitope
FLYGALLLA

SEQ ID NO: 141 - protein, 9 aa, Artificial Sequence
  Homo sapiens myelin proteolipid protein epitope
KLIETYFSK

SEQ ID NO: 142 - protein, 9 aa, Artificial Sequence
  Homo sapiens Myelin-associated glycoprotein precursor epitope
LMWAKIGPV

SEQ ID NO: 143 - protein, 9 aa, Artificial Sequence
  Homo sapiens Myelin-associated glycoprotein precursor epitope
SLLLELEEV

SEQ ID NO: 144 - protein, 9 aa, Artificial Sequence
  Homo sapiens Myelin-associated glycoprotein precursor epitope
VLFSSDFRI

SEQ ID NO: 145 - protein, 9 aa, Artificial Sequence
  Homo sapiens Myosin heavy chain, skeletal muscle, adult 2 (Myosin heavy chain IIa) (MyHC-IIa) epitope
EFQKMRRDL

SEQ ID NO: 146 - protein, 9 aa, Artificial Sequence
  Homo sapiens Myosin heavy chain, skeletal muscle, adult 2 (Myosin heavy chain IIa) (MyHC-IIa) epitope
KMRRDLEEA

SEQ ID NO: 147 - protein, 12 aa, Artificial Sequence
  Homo sapiens peroxiredoxin-2 isoform a epitope
EVKLSDYKGKYV

SEQ ID NO: 148 - protein, 10 aa, Artificial Sequence
  Homo sapiens proinsulin precursor epitope
HLCGSHLVEA

SEQ ID NO: 149 - protein, 10 aa, Artificial Sequence
  Homo sapiens proinsulin precursor epitope
ALWGPDPAAA

SEQ ID NO: 150 - protein, 9 aa, Artificial Sequence
  Homo sapiens proinsulin precursor epitope
RLLPLLALL

SEQ ID NO: 151 - protein, 10 aa, Artificial Sequence
  Homo sapiens proinsulin precursor epitope
ALWMRLLPLL

SEQ ID NO: 152 - protein, 10 aa, Artificial Sequence
  Homo sapiens proinsulin precursor epitope
WMRLLPLLAL

SEQ ID NO: 153 - protein, 10 aa, Artificial Sequence
  Homo sapiens proinsulin precursor epitope
PLALEGSLQK

SEQ ID NO: 154 - protein, 10 aa, Artificial Sequence
  Homo sapiens proinsulin precursor epitope
PLLALLALWG

SEQ ID NO: 155 - protein, 9 aa, Artificial Sequence
  Homo sapiens Receptor-type tyrosine-protein phosphatase-like N precursor epitope
LLPPLLEHL

SEQ ID NO: 156 - protein, 9 aa, Artificial Sequence
  Homo sapiens Receptor-type tyrosine-protein phosphatase-like N precursor epitope
SLAAGVKLL

SEQ ID NO: 157 - protein, 9 aa, Artificial Sequence
  Homo sapiens Receptor-type tyrosine-protein phosphatase-like N precursor epitope
SLSPLQAEL

SEQ ID NO: 158 - protein, 9 aa, Artificial Sequence
  Homo sapiens Receptor-type tyrosine-protein phosphatase-like N precursor epitope
ALTAVAEEV

SEQ ID NO: 159 - protein, 10 aa, Artificial Sequence
  Homo sapiens Receptor-type tyrosine-protein phosphatase-like N precursor epitope
SLYHVYEVNL

SEQ ID NO: 160 - protein, 9 aa, Artificial Sequence
  Homo sapiens Receptor-type tyrosine-protein phosphatase-like N precursor epitope
TIADFWQMV

SEQ ID NO: 161 - protein, 9 aa, Artificial Sequence
  Homo sapiens Receptor-type tyrosine-protein phosphatase-like N precursor epitope
VIVMLTPLV

SEQ ID NO: 162 - protein, 9 aa, Artificial Sequence
  Homo sapiens Receptor-type tyrosine-protein phosphatase-like N precursor epitope
MVWESGCTV

SEQ ID NO: 163 - protein, 14 aa, Artificial Sequence
  Homo sapiens S-arrestin epitope
FLGELTSSEVATEV

SEQ ID NO: 164 - protein, 20 aa, Artificial Sequence
  Homo sapiens S-arrestin epitope
FMSDKPLHLAVSLNKEIYFH

SEQ ID NO: 165 - protein, 15 aa, Artificial Sequence
  Homo sapiens S-arrestin epitope
GEAEEGKRDKNDVDE

SEQ ID NO: 166 - protein, 20 aa, Artificial Sequence
  Homo sapiens S-arrestin epitope
GEPIPVTVTVTNNTEKTVKK

SEQ ID NO: 167 - protein, 20 aa, Artificial Sequence
  Homo sapiens S-arrestin epitope
HPQPEDPAKESYQDANLVFE

SEQ ID NO: 168 - protein, 20 aa, Artificial Sequence
  Homo sapiens S-arrestin epitope
IKAFVEQVANVVLYSSDYYV

SEQ ID NO: 169 - protein, 20 aa, Artificial Sequence
  Homo sapiens S-arrestin epitope
KSSVRLLIRKVQHAPLEMGP

SEQ ID NO: 170 - protein, 20 aa, Artificial Sequence
  Homo sapiens S-arrestin epitope
QPRAEAAWQFFMSDKPLHLA

SEQ ID NO: 171 - protein, 20 aa, Artificial Sequence
  Homo sapiens S-arrestin epitope
SYQDANLVFEEFARHNLKDA

SEQ ID NO: 172 - protein, 20 aa, Artificial Sequence
  Homo sapiens S-arrestin epitope
TDAEEDKIPKKSSVRLLIRK

SEQ ID NO: 173 - protein, 20 aa, Artificial Sequence
  Homo sapiens S-arrestin epitope
TNNTEKTVKKIKAFVEQVAN

SEQ ID NO: 174 - protein, 20 aa, Artificial Sequence
  Homo sapiens S-arrestin epitope
VQHAPLEMGPQPRAEAAWQF

SEQ ID NO: 175 - protein, 20 aa, Artificial Sequence
  Homo sapiens S-arrestin epitope
VSLNKEIYFHGEPIPVTVTV

SEQ ID NO: 176 - protein, 20 aa, Artificial Sequence
  Homo sapiens S-arrestin epitope
VYVTLTCAFRYGQEDIDVIG

SEQ ID NO: 177 - protein, 20 aa, Artificial Sequence
  Homo sapiens S-arrestin epitope
YGQEDIDVIGLTFRRDLYFS

SEQ ID NO: 178 - protein, 10 aa, Artificial Sequence
  Homo sapiens SSA protein SS-56 epitope
YTCPLCRAPV

SEQ ID NO: 179 - protein, 8 aa, Artificial Sequence
  Homo sapiens Steroid 21-hydroxylase epitope
EPLARLEL

SEQ ID NO: 180 - protein, 20 aa, Artificial Sequence
  Homo sapiens Steroid 21-hydroxylase epitope
EPLARLELFVVLTRLLQAFT

SEQ ID NO: 181 - protein, 20 aa, Artificial Sequence
  Homo sapiens Steroid 21-hydroxylase epitope
IKDDNLMPAYYKCIQEVLKT

SEQ ID NO: 182 - protein, 20 aa, Artificial Sequence
  Homo sapiens Steroid 21-hydroxylase epitope
IRDSMEPVVEQLTQEFCERM

SEQ ID NO: 183 - protein, 8 aa, Artificial Sequence
  Homo sapiens T-cell receptor V beta chain 13.1 epitope
LGRAGLTY

SEQ ID NO: 184 - protein, 9 aa, Artificial Sequence
  Homo sapiens transaldolase 1 epitope
LLFSFAQAV

SEQ ID NO: 185 - protein, 9 aa, Artificial Sequence
  Homo sapiens Vasoactive intestinal polypeptide receptor 1 precursor epitope
RRKWRRWHL

SEQ ID NO: 186 - protein, 9 aa, Artificial Sequence
  Homo sapiens Vasoactive intestinal polypeptide receptor 1 precursor epitope
RRKWRRWHL

SEQ ID NO: 187 - protein, 20 aa, Artificial Sequence
  Arachis hypogaea 2S protein 1 epitope
AHASARQQWELQGDRRCQSQ

SEQ ID NO: 188 - protein, 20 aa, Artificial Sequence
  Arachis hypogaea 2S protein 1 epitope
AKLTILVALALFLLAAHASA

SEQ ID NO: 189 - protein, 19 aa, Artificial Sequence
  Arachis hypogaea 2S protein 1 epitope
ALQQIMENQSDRLQGRQQE

SEQ ID NO: 190 - protein, 20 aa, Artificial Sequence
  Arachis hypogaea 2S protein 1 epitope
ANLRPCEQHLMQKIQRDEDS

SEQ ID NO: 191 - protein, 20 aa, Artificial Sequence
  Arachis hypogaea 2S protein 1 epitope
CNELNEFENNQRCMCEALQQ

SEQ ID NO: 192 - protein, 16 aa, Artificial Sequence
  Homo sapiens 5-hydroxytryptamine receptor 2C (5-HT-2C) (Serotonin receptor 2C) (5-HT2C) (5-HTR2C) (5HT-1C) epitope
PRGTMQAINNERKASK

SEQ ID NO: 193 - protein, 12 aa, Artificial Sequence
  Bos taurus Allergen Bos d 2 precursor epitope
DQGTCLLLTEVA

SEQ ID NO: 194 - protein, 14 aa, Artificial Sequence
  Bos taurus Allergen Bos d 2 precursor epitope
ELEKYQQLNSERGV

SEQ ID NO: 195 - protein, 13 aa, Artificial Sequence
  Bos taurus Allergen Bos d 2 precursor epitope
GERITKMTEGLAK

SEQ ID NO: 196 - protein, 14 aa, Artificial Sequence
  Bos taurus Allergen Bos d 2 precursor epitope
PGEWRIIYAAADNK

SEQ ID NO: 197 - protein, 8 aa, Artificial Sequence
  Bos taurus Allergen Bos d 2 precursor epitope
RIECINDC

SEQ ID NO: 198 - protein, 12 aa, Artificial Sequence
  Bos taurus Allergen Bos d 2 precursor epitope
VAKRQEGYVYVL

SEQ ID NO: 199 - protein, 10 aa, Artificial Sequence
  Bos taurus Allergen Bos d 2 precursor epitope
VSENMLVTYV

SEQ ID NO: 200 - protein, 12 aa, Artificial Sequence
  Bos taurus Allergen Bos d 2 precursor epitope
DQGTCLLLTEVA

SEQ ID NO: 201 - protein, 14 aa, Artificial Sequence
  Bos taurus Allergen Bos d 2 precursor epitope
ELEKYQQLNSERGV

SEQ ID NO: 202 - protein, 16 aa, Artificial Sequence
  Bos taurus Allergen Bos d 2 precursor epitope
ELEKYQQLNSERGVPN

SEQ ID NO: 203 - protein, 13 aa, Artificial Sequence
  Bos taurus Allergen Bos d 2 precursor epitope
GERITKMTEGLAK

SEQ ID NO: 204 - protein, 14 aa, Artificial Sequence
  Bos taurus Allergen Bos d 2 precursor epitope
PGEWRIIYAAADNK

SEQ ID NO: 205 - protein, 8 aa, Artificial Sequence
  Bos taurus Allergen Bos d 2 precursor epitope
RIECINDC

SEQ ID NO: 206 - protein, 12 aa, Artificial Sequence
  Bos taurus Allergen Bos d 2 precursor epitope
VAKRQEGYVYVL

SEQ ID NO: 207 - protein, 10 aa, Artificial Sequence
  Bos taurus Allergen Bos d 2 precursor epitope
VSENMLVTYV

SEQ ID NO: 208 - protein, 13 aa, Artificial Sequence
  Cryptomeria japonica Allergen Cry j 2 epitope
DIFASKNFHLQKN

SEQ ID NO: 209 - protein, 13 aa, Artificial Sequence
  Cryptomeria japonica Allergen Cry j 2 epitope
GIIAAYQNPASWK

SEQ ID NO: 210 - protein, 12 aa, Artificial Sequence
  Cryptomeria japonica Allergen Cry j 2 epitope
KLTSGKIASCLN

SEQ ID NO: 211 - protein, 12 aa, Artificial Sequence
  Cryptomeria japonica Allergen Cry j 2 epitope
QFAKLTGFTLMG

SEQ ID NO: 212 - protein, 8 aa, Artificial Sequence
  Aspergillus fumigatus allergen I/a; Asp f I/a epitope
INQQLNPK

SEQ ID NO: 213 - protein, 15 aa, Artificial Sequence
  Aspergillus fumigatus allergen I/a; Asp f I/a epitope
INQQLNPKTNKWEDK

SEQ ID NO: 214 - protein, 11 aa, Artificial Sequence
  Aspergillus fumigatus allergen I/a; Asp f I/a epitope
LNPKTNKWEDK

SEQ ID NO: 215 - protein, 8 aa, Artificial Sequence
  Aspergillus fumigatus allergen I/a; Asp f I/a epitope
INQQLNPK

SEQ ID NO: 216 - protein, 15 aa, Artificial Sequence
  Aspergillus fumigatus allergen I/a; Asp f I/a epitope
INQQLNPKTNKWEDK

SEQ ID NO: 217 - protein, 11 aa, Artificial Sequence
  Aspergillus fumigatus allergen I/a; Asp f I/a epitope
LNPKTNKWEDK

SEQ ID NO: 218 - protein, 7 aa, Artificial Sequence
  Aspergillus fumigatus allergen I/a; Asp f I/a epitope
TNKWEDK

SEQ ID NO: 219 - protein, 12 aa, Artificial Sequence
  Aspergillus fumigatus allergen I/a; Asp f I/a epitope
LNPKTNKWEDKR

SEQ ID NO: 220 - protein, 15 aa, Artificial Sequence
  Dermatophagoides farinae Allergen Mag epitope
PRLSWHQYTKRDSRE

SEQ ID NO: 221 - protein, 15 aa, Artificial Sequence
  Dermatophagoides farinae Allergen Mag epitope
TVDLISPVTKRASLK

SEQ ID NO: 222 - protein, 18 aa, Artificial Sequence
  Bos taurus Alpha-S1-casein precursor epitope
AWYYVPLGTQYTDAPSFS

SEQ ID NO: 223 - protein, 18 aa, Artificial Sequence
  Bos taurus Alpha-S1-casein precursor epitope
DAYPSGAWYYVPLGTQYT

SEQ ID NO: 224 - protein, 18 aa, Artificial Sequence
  Bos taurus Alpha-S1-casein precursor epitope
DIGSESTEDQAMEDIKQM

SEQ ID NO: 225 - protein, 6 aa, Artificial Sequence
  Bos taurus Alpha-S1-casein precursor epitope
EDIKQM

SEQ ID NO: 226 - protein, 12 aa, Artificial Sequence
  Bos taurus Alpha-S1-casein precursor epitope
EPMIGVNQELAY

SEQ ID NO: 227 - protein, 18 aa, Artificial Sequence
  Bos taurus Alpha-S1-casein precursor epitope
EPMIGVNQELAYFYPELF

SEQ ID NO: 228 - protein, 17 aa, Artificial Sequence
  Arachis hypogaea Ara h 2.01 allergen epitope
ELNEFENNQRCMCEALQ

SEQ ID NO: 229 - protein, 16 aa, Artificial Sequence
  Arachis hypogaea Ara h 2.01 allergen epitope
SQLERANLRPCEQHLM

SEQ ID NO: 230 - protein, 15 aa, Artificial Sequence
  Cryptomeria japonica Cry j 1 precursor epitope
GATRDRPLWIIFSGN

SEQ ID NO: 231 - protein, 20 aa, Artificial Sequence
  Cryptomeria japonica Cry j 1 precursor epitope
IFSGNMNIKLKMPMYIAGYK

SEQ ID NO: 232 - protein, 20 aa, Artificial Sequence
  Cryptomeria japonica Cry j 1 precursor epitope
KMPMYIAGYKTFDGRGAQVY

SEQ ID NO: 233 - protein, 20 aa, Artificial Sequence
  Cryptomeria japonica Cry j 1 precursor epitope
LGHDDAYSDDKSMKVTVAFN

SEQ ID NO: 234 - protein, 19 aa, Artificial Sequence
  Cryptomeria japonica Cry j 1 precursor epitope
SGKYEGGNIYTKKEAFNVE

SEQ ID NO: 235 - protein, 11 aa, Artificial Sequence
  Cochliobolus lunatus Cytochrome c epitope
ENPKKYIPGTK

SEQ ID NO: 236 - protein, 11 aa, Artificial Sequence
  Cochliobolus lunatus Cytochrome c epitope
GLFGRKTGSVA

SEQ ID NO: 237 - protein, 9 aa, Artificial Sequence
  Cochliobolus lunatus Cytochrome c epitope
KIGPELHGL

SEQ ID NO: 238 - protein, 12 aa, Artificial Sequence
  Cochliobolus lunatus Cytochrome c epitope
LKAGEGNKIGPE

SEQ ID NO: 239 - protein, 11 aa, Artificial Sequence
  Cochliobolus lunatus Cytochrome c epitope
LKKPKDRNDLI

SEQ ID NO: 240 - protein, 18 aa, Artificial Sequence
  Dermatophagoides farinae Der f 2 allergen epitope
GLEIDVPGIDTNACHFVK

SEQ ID NO: 241 - protein, 20 aa, Artificial Sequence
  Dermatophagoides farinae Der f 2 allergen epitope
PGIDTNACHFVKCPLVKGQQ

SEQ ID NO: 242 - protein, 19 aa, Artificial Sequence
  Dermatophagoides pteronyssinus Der p 1 allergen epitope
RFGISNYCQIYPPNANKIR

SEQ ID NO: 243 - protein, 15 aa, Artificial Sequence
  Dermatophagoides pteronyssinus Der p 1 allergen epitope
AVNIVGYSNAQGVDY

SEQ ID NO: 244 - protein, 20 aa, Artificial Sequence
  Chironomus thummi globin Ctt 3-1 epitope
FAGKDLESIKGTAPFETHAN

SEQ ID NO: 245 - protein, 11 aa, Artificial Sequence
  Chironomus thummi globin Ctt 3-1 epitope
GTAPFETHANR

SEQ ID NO: 246 - protein, 21 aa, Artificial Sequence
  Chironomus thummi globin Ctt 3-1 epitope
KGTAPFETHANRIVGFFSKII

SEQ ID NO: 247 - protein, 21 aa, Artificial Sequence
  Chironomus thummi thummi Globin CTT-III epitope
AHTDFAGAEAAWGATLDTFFG

SEQ ID NO: 248 - protein, 20 aa, Artificial Sequence
  Chironomus thummi thummi Globin CTT-III epitope
FAGKDLESIKGTAPFEIHAN

SEQ ID NO: 249 - protein, 21 aa, Artificial Sequence
  Chironomus thummi thummi Globin CTT-III epitope
VNTFVASHKPRGVTHDQLNNF

SEQ ID NO: 250 - protein, 8 aa, Artificial Sequence
  Chironomus thummi thummi Globin CTT-III precursor epitope
ADPSIMAK

SEQ ID NO: 251 - protein, 21 aa, Artificial Sequence
  Chironomus thummi thummi Globin CTT-III precursor epitope
ADPSIMAKFTQFAGKDLESIK

SEQ ID NO: 252 - protein, 5 aa, Artificial Sequence
  Chironomus thummi thummi Globin CTT-III precursor epitope
AEAAW

SEQ ID NO: 253 - protein, 20 aa, Artificial Sequence
  Chironomus thummi thummi Globin CTT-III precursor epitope
AEAAWGATLDTFFGMIFSKM

SEQ ID NO: 254 - protein, 8 aa, Artificial Sequence
  Chironomus thummi thummi Globin CTT-III precursor epitope
AGFVSYMK

SEQ ID NO: 255 - protein, 15 aa, Artificial Sequence
  Phaseolus vulgaris Glycine-rich cell wall structural protein 1.8 precursor epitope
GGYGDGGAHGGGYGG

SEQ ID NO: 256 - protein, 15 aa, Artificial Sequence
  Phleum pratense Group V allergen Phl p 5 epitope
ATPEAKYDAYVATLS

SEQ ID NO: 257 - protein, 15 aa, Artificial Sequence
  Phleum pratense Group V allergen Phl p 5 epitope
FTVFEAAFNNAIKAG

SEQ ID NO: 258 - protein, 15 aa, Artificial Sequence
  Phleum pratense Group V allergen Phl p 5 epitope
KYDAYVATLSEALRI

SEQ ID NO: 259 - protein, 15 aa, Artificial Sequence
  Phleum pratense Group V allergen Phl p 5 epitope
PANDKFTVFEAAFNN

SEQ ID NO: 260 - protein, 15 aa, Artificial Sequence
  Phleum pratense Group V allergen Phl p 5 epitope
PKGGAESSSKAALTS

SEQ ID NO: 261 - protein, 16 aa, Artificial Sequence
  Homo sapiens KIAA1224 protein epitope
DLESYLQLNCERGTWR

SEQ ID NO: 262 - protein, 15 aa, Artificial Sequence
  Lepidoglyphus destructor Lep D 2 precursor epitope
KGEALDFNYGMTIPA

SEQ ID NO: 263 - protein, 12 aa, Artificial Sequence
  Corylus avellana lipid transfer protein precursor epitope
AGLPGKCGVNIP

SEQ ID NO: 264 - protein, 12 aa, Artificial Sequence
  Corylus avellana lipid transfer protein precursor epitope
AKGIAGLNPNLA

SEQ ID NO: 265 - protein, 12 aa, Artificial Sequence
  Corylus avellana lipid transfer protein precursor epitope
CGVNIPYKISPS

SEQ ID NO: 266 - protein, 12 aa, Artificial Sequence
  Corylus avellana lipid transfer protein precursor epitope
CKGVRAVNDASR

SEQ ID NO: 267 - protein, 12 aa, Artificial Sequence
  Corylus avellana lipid transfer protein precursor epitope
CVLYLKNGGVLP

SEQ ID NO: 268 - protein, 16 aa, Artificial Sequence
  Homo sapiens Lipocalin 1 (tear prealbumin) epitope
KPVRGVKLVGRDPKNN

SEQ ID NO: 269 - protein, 15 aa, Artificial Sequence
  Dermatophagoides farinae Mag3 epitope
EFNTEFTIHADKNNL

SEQ ID NO: 270 - protein, 15 aa, Artificial Sequence
  Dermatophagoides farinae Mag3 epitope
FTIHADKNNLKMHMD

SEQ ID NO: 271 - protein, 15 aa, Artificial Sequence
  Dermatophagoides farinae Mag3 epitope
KMHMDFPNVFQADLT

SEQ ID NO: 272 - protein, 13 aa, Artificial Sequence
  Apium graveolens Major allergen Api g 1 epitope
ALFKALEAYLIAN

SEQ ID NO: 273 - protein, 12 aa, Artificial Sequence
  Apium graveolens Major allergen Api g 1 epitope
DAVVPEENIKYA

SEQ ID NO: 274 - protein, 12 aa, Artificial Sequence
  Apium graveolens Major allergen Api g 1 epitope
DILLGFIESIEN

SEQ ID NO: 275 - protein, 12 aa, Artificial Sequence
  Apium graveolens Major allergen Api g 1 epitope
GGSICKTTAIFH

SEQ ID NO: 276 - protein, 12 aa, Artificial Sequence
  Apium graveolens Major allergen Api g 1 epitope
GVQTHVLELTSS

SEQ ID NO: 277 - protein, 15 aa, Artificial Sequence
  Aspergillus fumigatus Major allergen Asp f 2 precursor epitope
FGNRPTMEAVGAYDV

SEQ ID NO: 278 - protein, 15 aa, Artificial Sequence
  Aspergillus fumigatus Major allergen Asp f 2 precursor epitope
MEAVGAYDVIVNGDK

SEQ ID NO: 279 - protein, 16 aa, Artificial Sequence
  Canis lupus familiaris Major allergen Can f 1 precursor epitope
ALEDFREFSRAKGLNQ

SEQ ID NO: 280 - protein, 16 aa, Artificial Sequence
  Canis lupus familiaris Major allergen Can f 1 precursor epitope
DQEVPEKPDSVTPMIL

SEQ ID NO: 281 - protein, 12 aa, Artificial Sequence
  Corylus avellana major allergen Cor a 1.0401 epitope
AGKEKAAGLFKA

SEQ ID NO: 282 - protein, 12 aa, Artificial Sequence
  Corylus avellana major allergen Cor a 1.0401 epitope
AGLFKAVEAYLL

SEQ ID NO: 283 - protein, 12 aa, Artificial Sequence
  Corylus avellana major allergen Cor a 1.0401 epitope
APQHFTSAENLE

SEQ ID NO: 284 - protein, 12 aa, Artificial Sequence
  Corylus avellana major allergen Cor a 1.0401 epitope
ARLFKSFVLDAD

SEQ ID NO: 285 - protein, 12 aa, Artificial Sequence
  Corylus avellana major allergen Cor a 1.0401 epitope
EIDHANFKYCYS

SEQ ID NO: 286 - protein, 13 aa, Artificial Sequence
  Daucus carota Major allergen Dau c 1 epitope
ALFKAIEAYLIAN

SEQ ID NO: 287 - protein, 16 aa, Artificial Sequence
  Equus caballus Major allergen Equ c 1 precursor epitope
DGYNVFRISEFENDEH

SEQ ID NO: 288 - protein, 16 aa, Artificial Sequence
  Equus caballus Major allergen Equ c 1 precursor epitope
DKDRPFQLFEFYAREP

SEQ ID NO: 289 - protein, 16 aa, Artificial Sequence
  Equus caballus Major allergen Equ c 1 precursor epitope
DLTKIDRCFQLRGNGV

SEQ ID NO: 290 - protein, 16 aa, Artificial Sequence
  Equus caballus Major allergen Equ c 1 precursor epitope
DRPFQLFEFYAREPDV

SEQ ID NO: 291 - protein, 16 aa, Artificial Sequence
  Equus caballus Major allergen Equ c 1 precursor epitope
DVSPEIKEEFVKIVQK

SEQ ID NO: 292 - protein, 17 aa, Artificial Sequence
  Felis catus major allergen I epitope
ENARILKNCVDAKMTEE

SEQ ID NO: 293 - protein, 17 aa, Artificial Sequence
  Felis catus major allergen I epitope
RDVDLFLTGTPDEYVEQ

SEQ ID NO: 294 - protein, 17 aa, Artificial Sequence
  Felis catus major allergen I epitope
TGTPDEYVEQVAQYKAL

SEQ ID NO: 295 - protein, 17 aa, Artificial Sequence
  Felis catus Major allergen I polypeptide chain 1 precursor epitope
DVDLFLTGTPDEYVEQV

SEQ ID NO: 296 - protein, 17 aa, Artificial Sequence
  Felis catus Major allergen I polypeptide chain 1 precursor epitope
EICPAVKRDVDLFLTGT

SEQ ID NO: 297 - protein, 16 aa, Artificial Sequence
  Felis catus Major allergen I polypeptide chain 1 precursor epitope
EQVAQYKALPVVLENA

SEQ ID NO: 298 - protein, 17 aa, Artificial Sequence
  Felis catus Major allergen I polypeptide chain 1 precursor epitope
KALPVVLENARILKNCV

SEQ ID NO: 299 - protein, 17 aa, Artificial Sequence
  Felis catus Major allergen I polypeptide chain 1 precursor epitope
LFLTGTPDEYVEQVAQY

SEQ ID NO: 300 - protein, 16 aa, Artificial Sequence
  Felis catus major allergen I, polypeptide chain 1 epitope
KENALSLLDKIYTSPL

SEQ ID NO: 301 - protein, 16 aa, Artificial Sequence
  Felis catus major allergen I, polypeptide chain 1 epitope
KMTEEDKENALSLLDK

SEQ ID NO: 302 - protein, 15 aa, Artificial Sequence
  Malus x domestica Major allergen Mal d 1 epitope
GLFKLIESYLKDHPD

SEQ ID NO: 303 - protein, 15 aa, Artificial Sequence
  Prunus avium Major allergen Pru av 1 epitope
NLFKLIETYLKGHPD

SEQ ID NO: 304 - protein, 20 aa, Artificial Sequence
  Hevea brasiliensis Major latex allergen Hev b 5 epitope
AAPAEGEKPAEEEKPITEAA

SEQ ID NO: 305 - protein, 20 aa, Artificial Sequence
  Hevea brasiliensis Major latex allergen Hev b 5 epitope
AEEEKPITEAAETATTEVPV

SEQ ID NO: 306 - protein, 20 aa, Artificial Sequence
  Hevea brasiliensis Major latex allergen Hev b 5 epitope
APAEPEAPAPETEKAEEVEK

SEQ ID NO: 307 - protein, 20 aa, Artificial Sequence
  Hevea brasiliensis Major latex allergen Hev b 5 epitope
APEADQTTPEEKPAEPEPVA

SEQ ID NO: 308 - protein, 20 aa, Artificial Sequence
  Hevea brasiliensis Major latex allergen Hev b 5 epitope
ASEQETADATPEKEEPTAAP

SEQ ID NO: 309 - protein, 11 aa, Artificial Sequence
  Dermatophagoides pteronyssinus Major mite fecal allergen Der p 1 epitope
YAYVAREQSCR

SEQ ID NO: 310 - protein, 19 aa, Artificial Sequence
  Dermatophagoides pteronyssinus Major mite fecal allergen Der p 1 epitope
ALAQTHTAIAVIIGIKDLD

SEQ ID NO: 311 - protein, 35 aa, Artificial Sequence
  Olea europaea Major pollen allergen epitope
EDIPQPPVSQFHIQGQVYCDTCRAGFITELSEFIP

SEQ ID NO: 312 - protein, 31 aa, Artificial Sequence
  Olea europaea Major pollen allergen epitope
GASLRLQCKDKENGDVTFTEVGYTRAEGLYS

SEQ ID NO: 313 - protein, 34 aa, Artificial Sequence
  Olea europaea Major pollen allergen epitope
GTTRTVNPLGFFKKEALPKCAQVYNKLGMYPPNM

SEQ ID NO: 314 - protein, 53 aa, Artificial Sequence
  Olea europaea Major pollen allergen epitope
LVERDHKNEFCEITLISSGRKDCNEIPTEGWAKPSLKFKLNTVNGTTRTVNPL

SEQ ID NO: 315 - protein, 33 aa, Artificial Sequence
  Olea europaea Major pollen allergen epitope
MLVERDHKNEFCEITLISSGRKDCNEIPTEGWA

SEQ ID NO: 316 - protein, 12 aa, Artificial Sequence
  Artemisia vulgaris Major pollen allergen Art v 1 precursor epitope
AGGSPSPPADGG

SEQ ID NO: 317 - protein, 12 aa, Artificial Sequence
  Artemisia vulgaris Major pollen allergen Art v 1 precursor epitope
AGSKLCEKTSKT

SEQ ID NO: 318 - protein, 12 aa, Artificial Sequence
  Artemisia vulgaris Major pollen allergen Art v 1 precursor epitope
CDKKCIEWEKAQ

SEQ ID NO: 319 - protein, 12 aa, Artificial Sequence
  Artemisia vulgaris Major pollen allergen Art v 1 precursor epitope
DGGSPPPPADGG

SEQ ID NO: 320 - protein, 12 aa, Artificial Sequence
  Artemisia vulgaris Major pollen allergen Art v 1 precursor epitope
EKTSKTYSGKCD

SEQ ID NO: 321 - protein, 12 aa, Artificial Sequence
  Betula pendula Major pollen allergen Bet v 1-A epitope
AARLFKAFILDG

SEQ ID NO: 322 - protein, 15 aa, Artificial Sequence
  Betula pendula Major pollen allergen Bet v 1-A epitope
AARLFKAFILDGDNL

SEQ ID NO: 323 - protein, 12 aa, Artificial Sequence
  Betula pendula Major pollen allergen Bet v 1-A epitope
AEQVKASKEMGE

SEQ ID NO: 324 - protein, 21 aa, Artificial Sequence
  Betula pendula Major pollen allergen Bet v 1-A epitope
AFILDGDNLFPKVAPQAISSV

SEQ ID NO: 325 - protein, 12 aa, Artificial Sequence
  Betula pendula Major pollen allergen Bet v 1-A epitope
AISSVENIEGNG

SEQ ID NO: 326 - protein, 15 aa, Artificial Sequence
  Betula pendula Major pollen allergen Bet v 1-A epitope
ETLLRAVESYLLAHS

SEQ ID NO: 327 - protein, 16 aa, Artificial Sequence
  Betula pendula Major pollen allergen Bet v 1-F/I epitope
GETLLRAVESYLLAHS

SEQ ID NO: 328 - protein, 20 aa, Artificial Sequence
  Chamaecyparis obtusa Major pollen allergen Cha o 1 precursor epitope
ANNNYDPWSIYAIGGSSNPT

SEQ ID NO: 329 - protein, 20 aa, Artificial Sequence
  Chamaecyparis obtusa Major pollen allergen Cha o 1 precursor epitope
ASTGVTISNNHFFNHHKVML

SEQ ID NO: 330 - protein, 20 aa, Artificial Sequence
  Chamaecyparis obtusa Major pollen allergen Cha o 1 precursor epitope
CANWVWRSTQDSFNNGAYFV

SEQ ID NO: 331 - protein, 20 aa, Artificial Sequence
  Chamaecyparis obtusa Major pollen allergen Cha o 1 precursor epitope
DAITMRNVTDVWIDHNSLSD

SEQ ID NO: 332 - protein, 20 aa, Artificial Sequence
  Chamaecyparis obtusa Major pollen allergen Cha o 1 precursor epitope
DANWDQNRMKLADCAVGFGS

SEQ ID NO: 333 - protein, 20 aa, Artificial Sequence
  Cynodon dactylon Major pollen allergen Cyn d 1 epitope
AIGDKPGPNITATYGNKWLE

SEQ ID NO: 334 - protein, 20 aa, Artificial Sequence
  Cynodon dactylon Major pollen allergen Cyn d 1 epitope
CYEIKCKEPVECSGEPVLVK

SEQ ID NO: 335 - protein, 20 aa, Artificial Sequence
  Cynodon dactylon Major pollen allergen Cyn d 1 epitope
DHGGACGYKDVDKPPFDGMT

SEQ ID NO: 336 - protein, 20 aa, Artificial Sequence
  Cynodon dactylon Major pollen allergen Cyn d 1 epitope
EGGAHLVQDDVIPANWKPDT

SEQ ID NO: 337 - protein, 20 aa, Artificial Sequence
  Cynodon dactylon Major pollen allergen Cyn d 1 epitope
FKDGLGCGACYEIKCKEPVE

SEQ ID NO: 338 - protein, 15 aa, Artificial Sequence
  Phleum pratense Major pollen allergen Phl p 4 precursor epitope
FAEYKSDYVYQPFPK

SEQ ID NO: 339 - protein, 15 aa, Artificial Sequence
  Phleum pratense Major pollen allergen Phl p 4 precursor epitope
MLLRKYGIAAENVID

SEQ ID NO: 340 - protein, 15 aa, Artificial Sequence
  Phleum pratense Major pollen allergen Phl p 4 precursor epitope
NSFKPFAEYKSDYVY

SEQ ID NO: 341 - protein, 20 aa, Artificial Sequence
  Rattus norvegicus Major urinary protein precursor epitope
ASNKREKIEENGSMRVFMQH

SEQ ID NO: 342 - protein, 20 aa, Artificial Sequence
  Rattus norvegicus Major urinary protein precursor epitope
DIKEKFAKLCEAHGITRDNI

SEQ ID NO: 343 - protein, 20 aa, Artificial Sequence
  Rattus norvegicus Major urinary protein precursor epitope
EEASSTRGNLDVAKLNGDWF

SEQ ID NO: 344 - protein, 20 aa, Artificial Sequence
  Rattus norvegicus Major urinary protein precursor epitope
EENGSMRVFMQHIDVLENSL

SEQ ID NO: 345 - protein, 20 aa, Artificial Sequence
  Rattus norvegicus Major urinary protein precursor epitope
ENSLGFKFRIKENGECRELY

SEQ ID NO: 346 - protein, 21 aa, Artificial Sequence
  Dermatophagoides farinae Mite group 2 allergen Der f 2 precursor epitope
DIKYTWNVPKIAPKSENVVVT

SEQ ID NO: 347 - protein, 17 aa, Artificial Sequence
  Dermatophagoides farinae Mite group 2 allergen Der f 2 precursor epitope
DNGVLACAIATHGKIRD

SEQ ID NO: 348 - protein, 21 aa, Artificial Sequence
  Dermatophagoides farinae Mite group 2 allergen Der f 2 precursor epitope
EALFDANQNTKTAKIEIKASL

SEQ ID NO: 349 - protein, 45 aa, Artificial Sequence
  Dermatophagoides farinae Mite group 2 allergen Der f 2 precursor epitope
QYDIKYTWNVPKIAPKSENVVVTVKLIGDNGVLACAIATHGKIRD

SEQ ID NO: 350 - protein, 19 aa, Artificial Sequence
  Dermatophagoides farinae Mite group 2 allergen Der f 2 precursor epitope
TKTAKIEIKASLDGLEIDV

SEQ ID NO: 351 - protein, 14 aa, Artificial Sequence
  Dermatophagoides pteronyssinus Mite group 2 allergen Der p 2 epitope
ASIDGLGVDVPGID

SEQ ID NO: 352 - protein, 15 aa, Artificial Sequence
  Dermatophagoides pteronyssinus Mite group 2 allergen Der p 2 epitope
FEAVQNTKTAKIEIK

SEQ ID NO: 353 - protein, 17 aa, Artificial Sequence
  Dermatophagoides pteronyssinus Mite group 2 allergen Der p 2 epitope
RGKPPQLEAVFEAVQNT

SEQ ID NO: 354 - protein, 15 aa, Artificial Sequence
  Dermatophagoides pteronyssinus Mite group 2 allergen Der p 2 precursor epitope
CHGSEPCIIHRGKPF

SEQ ID NO: 355 - protein, 27 aa, Artificial Sequence
  Dermatophagoides pteronyssinus Mite group 2 allergen Der p 2 precursor epitope
CPLVKGQQYDIKYTWNVPKIAPKSENV

SEQ ID NO: 356 - protein, 26 aa, Artificial Sequence
  Dermatophagoides pteronyssinus Mite group 2 allergen Der p 2 precursor epitope
DIKYTWNVPKIAPKSENVVVTVKVMG

SEQ ID NO: 357 - protein, 15 aa, Artificial Sequence
  Dermatophagoides pteronyssinus Mite group 2 allergen Der p 2 precursor epitope
DQVDVKDCANHEIKK

SEQ ID NO: 358 - protein, 20 aa, Artificial Sequence
  Dermatophagoides pteronyssinus Mite group 2 allergen Der p 2 precursor epitope
DQVDVKDCANHEIKKVLVPG

SEQ ID NO: 359 - protein, 15 aa, Artificial Sequence
  Dermatophagoides pteronyssinus Mite group 2 allergen Der p 2 precursor epitope
CHGSEPCIIHRGKPF

SEQ ID NO: 360 - protein, 15 aa, Artificial Sequence
  Lepidoglyphus destructor Mite group 2 allergen Lep d 2 precursor epitope
DHGVMACGTVHGQVE

SEQ ID NO: 361 - protein, 15 aa, Artificial Sequence
  Lepidoglyphus destructor Mite group 2 allergen Lep d 2 precursor epitope
GCKFIKCPVKKGEAL

SEQ ID NO: 362 - protein, 15 aa, Artificial Sequence
  Lepidoglyphus destructor Mite group 2 allergen Lep d 2 precursor epitope
GEKMTLEAKFAANQD

SEQ ID NO: 363 - protein, 15 aa, Artificial Sequence
  Lepidoglyphus destructor Mite group 2 allergen Lep d 2 precursor epitope
GEVTELDITGCSGDT

SEQ ID NO: 364 - protein, 15 aa, Artificial Sequence
  Lepidoglyphus destructor Mite group 2 allergen Lep d 2 precursor epitope
GKMTFKDCGHGEVTE

SEQ ID NO: 365 - protein, 16 aa, Artificial Sequence
  Homo sapiens Neurofilament heavy polypeptide (NF-H) (Neurofilament triplet H protein) (200 kDa neurofilament protein) epitope
YQEAIQQLDAELRNTK

SEQ ID NO: 366 - protein, 10 aa, Artificial Sequence
  Prunus persica Non-specific lipid-transfer protein 1 epitope
AAALPGKCGV

SEQ ID NO: 367 - protein, 10 aa, Artificial Sequence
  Prunus persica Non-specific lipid-transfer protein 1 epitope
ACCNGIRNVN

SEQ ID NO: 368 - protein, 10 aa, Artificial Sequence
  Prunus persica Non-specific lipid-transfer protein 1 epitope
APCIPYVRGG

SEQ ID NO: 369 - protein, 10 aa, Artificial Sequence
  Prunus persica Non-specific lipid-transfer protein 1 epitope
IRNVNNLART

SEQ ID NO: 370 - protein, 11 aa, Artificial Sequence
  Prunus persica Non-specific lipid-transfer protein 1 epitope
ISASTNCATVK

SEQ ID NO: 371 - protein, 10 aa, Artificial Sequence
  Prunus persica Non-specific lipid-transfer protein 1 epitope
NLARTTPDRQ

SEQ ID NO: 372 - protein, 10 aa, Artificial Sequence
  Gallus gallus Ovalbumin epitope
CFDVFKELKV

SEQ ID NO: 373 - protein, 10 aa, Artificial Sequence
  Gallus gallus Ovalbumin epitope
GSIGAASMEF

SEQ ID NO: 374 - protein, 18 aa, Artificial Sequence
  Gallus gallus Ovalbumin epitope
IGLFRVASMASEKMKILE

SEQ ID NO: 375 - protein, 18 aa, Artificial Sequence
  Gallus gallus Ovalbumin epitope
IKHIATNAVLFFGRCVSP

SEQ ID NO: 376 - protein, 13 aa, Artificial Sequence
  Gallus gallus Ovalbumin epitope
IMSALAMVYLGAK

SEQ ID NO: 377 - protein, 14 aa, Artificial Sequence
  Gallus gallus Ovomucoid precursor epitope
AEVDCSRFPNATDK

SEQ ID NO: 378 - protein, 14 aa, Artificial Sequence
  Gallus gallus Ovomucoid precursor epitope
ATDKEGKDVLVCNK

SEQ ID NO: 379 - protein, 17 aa, Artificial Sequence
  Gallus gallus Ovomucoid precursor epitope
AVVESNGTLTLSHFGKC

SEQ ID NO: 380 - protein, 16 aa, Artificial Sequence
  Gallus gallus Ovomucoid precursor epitope
CLLCAYSIEFGTNISK

SEQ ID NO: 381 - protein, 20 aa, Artificial Sequence
  Gallus gallus Ovomucoid precursor epitope
DNECLLCAHKVEQGASVDKR

SEQ ID NO: 382 - protein, 16 aa, Artificial Sequence
  Musa acuminata pectate lyase epitope
GHSDELTSDKSMQVTI

SEQ ID NO: 383 - protein, 16 aa, Artificial Sequence
  Zinnia violacea Pectate lyase precursor epitope
GHSDSYTQDKNMQVTI

SEQ ID NO: 384 - protein, 21 aa, Artificial Sequence
  Dermatophagoides farinae Peptidase 1 precursor (Major mite fecal allergen Der f 1) (Allergen Der f I) epitope
DGRTIIQHDNGYQPNYHAVNI

SEQ ID NO: 385 - protein, 19 aa, Artificial Sequence
  Dermatophagoides farinae Peptidase 1 precursor (Major mite fecal allergen Der f 1) (Allergen Der f I) epitope
DLRSLRTVTPIRMQGGCGS

SEQ ID NO: 386 - protein, 19 aa, Artificial Sequence
  Dermatophagoides farinae Peptidase 1 precursor (Major mite fecal allergen Der f 1) (Allergen Der f I) epitope
GCGSCWAFSGVAATESAYL

SEQ ID NO: 387 - protein, 21 aa, Artificial Sequence
  Dermatophagoides farinae Peptidase 1 precursor (Major mite fecal allergen Der f 1) (Allergen Der f I) epitope
IREALTQTHTAIAVIIGIKDL

SEQ ID NO: 388 - protein, 19 aa, Artificial Sequence
  Dermatophagoides farinae Peptidase 1 precursor (Major mite fecal allergen Der f 1) (Allergen Der f I) epitope
IRMQGGCGSCWAFSGVAAT

SEQ ID NO: 389 - protein, 19 aa, Artificial Sequence
  Euroglyphus maynei Peptidase 1 precursor (Mite group 1 allergen Eur m 1) (Allergen Eur m I) epitope
FRHYDGRTIMQHDNGYQPN

SEQ ID NO: 390 - protein, 19 aa, Artificial Sequence
  Euroglyphus maynei Peptidase 1 precursor (Mite group 1 allergen Eur m 1) (Allergen Eur m I) epitope
GRTIMQHDNGYQPNYHAVN

SEQ ID NO: 391 - protein, 19 aa, Artificial Sequence
  Euroglyphus maynei Peptidase 1 precursor (Mite group 1 allergen Eur m 1) (Allergen Eur m I) epitope
HAVNIVGYGNTQGVDYWIV

SEQ ID NO: 392 - protein, 19 aa, Artificial Sequence
  Euroglyphus maynei Peptidase 1 precursor (Mite group 1 allergen Eur m 1) (Allergen Eur m I) epitope
NKIRQALTQTHTAVAVIIG

SEQ ID NO: 393 - protein, 19 aa, Artificial Sequence
  Euroglyphus maynei Peptidase 1 precursor (Mite group 1 allergen Eur m 1) (Allergen Eur m I) epitope
PYVAREQSCHRPNAQRYGL

SEQ ID NO: 394 - protein, 15 aa, Artificial Sequence
  Phleum pratense Phl p 3 allergen epitope
AVQVTFTVQKGSDPK

SEQ ID NO: 395 - protein, 15 aa, Artificial Sequence
  Phleum pratense Phl p 3 allergen epitope
EEWEPLTKKGNVWEV

SEQ ID NO: 396 - protein, 15 aa, Artificial Sequence
  Phleum pratense Phl p 3 allergen epitope
FTVQKGSDPKKLVLD

SEQ ID NO: 397 - protein, 15 aa, Artificial Sequence
  Phleum pratense Phl p 3 allergen epitope
FTVQKGSDPKKLVLN

SEQ ID NO: 398 - protein, 15 aa, Artificial Sequence
  Phleum pratense Phl p 3 allergen epitope
GSDPKKLVLDIKYTR

SEQ ID NO: 399 - protein, 15 aa, Artificial Sequence
  Apis mellifera Phospholipase A2 precursor epitope
CDCDDKFYDCLKNSA

SEQ ID NO: 400 - protein, 12 aa, Artificial Sequence
  Apis mellifera Phospholipase A2 precursor epitope
CLHYTVDKSKPK

SEQ ID NO: 401 - protein, 15 aa, Artificial Sequence
  Apis mellifera Phospholipase A2 precursor epitope
CRTHDMCPDVMSAGE

SEQ ID NO: 402 - protein, 18 aa, Artificial Sequence
  Apis mellifera Phospholipase A2 precursor epitope
DTISSYFVGKMYFNLIDT

SEQ ID NO: 403 - protein, 18 aa, Artificial Sequence
  Apis mellifera Phospholipase A2 precursor epitope
ERTEGRCLHYTVDKSKPK

SEQ ID NO: 404 - protein, 16 aa, Artificial Sequence
  Spiroplasma citri plectrovirus spv1-r8a2b orf 14 transmembrane protein epitope
HVIEVQQINSERSWFF

SEQ ID NO: 405 - protein, 20 aa, Artificial Sequence
  Lolium perenne pollen allergen epitope
CGYKDVDKAPFNGMTGCGNT

SEQ ID NO: 406 - protein, 20 aa, Artificial Sequence
  Lolium perenne pollen allergen epitope
GAGPKDNGGACGYKDVDKAP

SEQ ID NO: 407 - protein, 20 aa, Artificial Sequence
  Lolium perenne pollen allergen epitope
SEVEDVIPEGWKADTSYSAK

SEQ ID NO: 408 - protein, 20 aa, Artificial Sequence
  Lolium perenne pollen allergen epitope
VEKGSNPNYLAILVKYVDGD

SEQ ID NO: 409 - protein, 20 aa, Artificial Sequence
  Lolium perenne pollen allergen epitope
YPDDTKPTFHVEKGSNPNYL

SEQ ID NO: 410 - protein, 16 aa, Artificial Sequence
  Ambrosia artemisiifolia Pollen allergen Amb a 1.1 precursor epitope
GAGDENIEDRGMLATV

SEQ ID NO: 411 - protein, 16 aa, Artificial Sequence
  Ambrosia artemisiifolia Pollen allergen Amb a 1.1 precursor epitope
GAGDENIEDRGMLATV

SEQ ID NO: 412 - protein, 16 aa, Artificial Sequence
  Ambrosia artemisiifolia Pollen allergen Amb a 2 precursor epitope
GASDTHFQDLKMHVTL

SEQ ID NO: 413 - protein, 16 aa, Artificial Sequence
  Ambrosia artemisiifolia Pollen allergen Amb a 2 precursor epitope
GASDTHFQDLKMHVTL

SEQ ID NO: 414 - protein, 11 aa, Artificial Sequence
  Ambrosia artemisiifolia var. elatior Pollen allergen Amb a 3 epitope
EEAYHACDIKD

SEQ ID NO: 415 - protein, 15 aa, Artificial Sequence
  Ambrosia artemisiifolia var. elatior Pollen allergen Amb a 3 epitope
GKVYLVGGPELGGWK

SEQ ID NO: 416 - protein, 15 aa, Artificial Sequence
  Ambrosia artemisiifolia var. elatior Pollen allergen Amb a 3 epitope
LGGWKLQSDPRAYAL

SEQ ID NO: 417 - protein, 15 aa, Artificial Sequence
  Ambrosia artemisiifolia var. elatior Pollen allergen Amb a 3 epitope
PGGPDRFTLLTPGSH

SEQ ID NO: 418 - protein, 15 aa, Artificial Sequence
  Ambrosia artemisiifolia var. elatior Pollen allergen Amb a 5 epitope
AYCCSDPGRYCPWQV

SEQ ID NO: 419 - protein, 20 aa, Artificial Sequence
  Ambrosia artemisiifolia var. elatior Pollen allergen Amb a 5 epitope
CGEKRAYCCSDPGRYCPWQV

SEQ ID NO: 420 - protein, 17 aa, Artificial Sequence
  Ambrosia artemisiifolia var. elatior Pollen allergen Amb a 5 epitope
DPGRYCPWQVVCYESSE

SEQ ID NO: 421 - protein, 20 aa, Artificial Sequence
  Ambrosia artemisiifolia var. elatior Pollen allergen Amb a 5 epitope
DPGRYCPWQVVCYESSEICS

SEQ ID NO: 422 - protein, 15 aa, Artificial Sequence
  Ambrosia artemisiifolia var. elatior Pollen allergen Amb a 5 epitope
GNVCGEKRAYCCSDP

SEQ ID NO: 423 - protein, 15 aa, Artificial Sequence
  Ambrosia artemisiifolia var. elatior Pollen allergen Amb a 5 epitope
LVPCAWAGNVCGEKR

SEQ ID NO: 424 - protein, 20 aa, Artificial Sequence
  Ambrosia artemisiifolia var. elatior Pollen allergen Amb a 5 epitope
LVPCAWAGNVCGEKRAYCCS

SEQ ID NO: 425 - protein, 15 aa, Artificial Sequence
  Ambrosia artemisiifolia var. elatior Pollen allergen Amb a 5 epitope
VCYESSEICSKKCGK

SEQ ID NO: 426 - protein, 20 aa, Artificial Sequence
  Ambrosia trifida Pollen allergen Amb t 5 precursor epitope
CGKVGKYCCSPIGKYCVCYD

SEQ ID NO: 427 - protein, 20 aa, Artificial Sequence
  Ambrosia trifida Pollen allergen Amb t 5 precursor epitope
DDGLCYEGTNCGKVGKYCCS

SEQ ID NO: 428 - protein, 12 aa, Artificial Sequence
  Ambrosia trifida Pollen allergen Amb t 5 precursor epitope
GKYCVCYDSKAI

SEQ ID NO: 429 - protein, 14 aa, Artificial Sequence
  Ambrosia trifida Pollen allergen Amb t 5 precursor epitope
PIGKYCVCYDSKAI

SEQ ID NO: 430 - protein, 20 aa, Artificial Sequence
  Ambrosia trifida Pollen allergen Amb t 5 precursor epitope
PIGKYCVCYDSKAICNKNCT

SEQ ID NO: 431 - protein, 14 aa, Artificial Sequence
  Ambrosia trifida Pollen allergen Amb t 5 precursor epitope
VCYDSKAICNKNCT

SEQ ID NO: 432 - protein, 15 aa, Artificial Sequence
  Betula pendula pollen allergen Bet v 1 epitope
HEVKAEQVKATKEMG

SEQ ID NO: 433 - protein, 20 aa, Artificial Sequence
  Poa pratensis Pollen allergen KBG 60 precursor epitope
AANKYKTFVATFGAASNKAF

SEQ ID NO: 434 - protein, 20 aa, Artificial Sequence
  Poa pratensis Pollen allergen KBG 60 precursor epitope
AAPANDKFTVFEAAFNDAIK

SEQ ID NO: 435 - protein, 20 aa, Artificial Sequence
  Poa pratensis Pollen allergen KBG 60 precursor epitope
AAVDSSKAALTSKLDAAYKL

SEQ ID NO: 436 - protein, 20 aa, Artificial Sequence
  Poa pratensis Pollen allergen KBG 60 precursor epitope
AEEVKATPAGELQVIDKVDA

SEQ ID NO: 437 - protein, 20 aa, Artificial Sequence
  Poa pratensis Pollen allergen KBG 60 precursor epitope
AFKVAATAANAAPANDKFTV

SEQ ID NO: 438 - protein, 20 aa, Artificial Sequence
  Lolium perenne Pollen allergen Lol p 1 precursor epitope
AFGSMAKKGEEQNVRSAGEL

SEQ ID NO: 439 - protein, 20 aa, Artificial Sequence
  Lolium perenne Pollen allergen Lol p 1 precursor epitope
AGELELQFRRVKCKYPDDTK

SEQ ID NO: 440 - protein, 20 aa, Artificial Sequence
  Lolium perenne Pollen allergen Lol p 1 precursor epitope
AKSTWYGKPTGAGPKDNGGA

SEQ ID NO: 441 - protein, 20 aa, Artificial Sequence
  Lolium perenne Pollen allergen Lol p 1 precursor epitope
APYHFDLSGHAFGSMAKKGE

SEQ ID NO: 442 - protein, 12 aa, Artificial Sequence
  Lolium perenne Pollen allergen Lol p 1 precursor epitope
IAPYHFDLSGHA

SEQ ID NO: 443 - protein, 20 aa, Artificial Sequence
  Lolium perenne Pollen allergen Lol p VA precursor epitope
AALTKAITAMTQAQKAGKPA

SEQ ID NO: 444 - protein, 20 aa, Artificial Sequence
  Lolium perenne Pollen allergen Lol p VA precursor epitope
AANAAPTNDKFTVFESAFNK

SEQ ID NO: 445 - protein, 20 aa, Artificial Sequence
  Lolium perenne Pollen allergen Lol p VA precursor epitope
ADKFKIFEAAFSESSKGLLA

SEQ ID NO: 446 - protein, 20 aa, Artificial Sequence
  Lolium perenne Pollen allergen Lol p VA precursor epitope
AFSESSKGLLATSAAKAPGL

SEQ ID NO: 447 - protein, 20 aa, Artificial Sequence
  Lolium perenne Pollen allergen Lol p VA precursor epitope
AYAATVAAAPEVKYAVFEAA

SEQ ID NO: 448 - protein, 12 aa, Artificial Sequence
  Phleum pratense Pollen allergen Phl p 1 epitope
ACSGEPVVVHIT

SEQ ID NO: 449 - protein, 12 aa, Artificial Sequence
  Phleum pratense Pollen allergen Phl p 1 epitope
AEDVIPEGWKAD

SEQ ID NO: 450 - protein, 12 aa, Artificial Sequence
  Phleum pratense Pollen allergen Phl p 1 epitope
AGELELQFRRVK

SEQ ID NO: 451 - protein, 12 aa, Artificial Sequence
  Phleum pratense Pollen allergen Phl p 1 epitope
DKWIELKESWGA

SEQ ID NO: 452 - protein, 12 aa, Artificial Sequence
  Phleum pratense Pollen allergen Phl p 1 epitope
DKWLDAKSTWYG

SEQ ID NO: 453 - protein, 12 aa, Artificial Sequence
  Phleum pratense Pollen allergen Phl p 1 precursor epitope
FEIKCTKPEACS

SEQ ID NO: 454 - protein, 12 aa, Artificial Sequence
  Phleum pratense Pollen allergen Phl p 1 precursor epitope
YHFDLSGHAFGA

SEQ ID NO: 455 - protein, 15 aa, Artificial Sequence
  Phleum pratense Pollen allergen Phl p 1 precursor epitope
ELKESWGAIWRIDTP

SEQ ID NO: 456 - protein, 15 aa, Artificial Sequence
  Phleum pratense Pollen allergen Phl p 1 precursor epitope
EPIAPYHFDLSGHAF

SEQ ID NO: 457 - protein, 15 aa, Artificial Sequence
  Phleum pratense Pollen allergen Phl p 1 precursor epitope
FEIKCTKPEACSGEP

SEQ ID NO: 458 - protein, 15 aa, Artificial Sequence
  Phleum pratense Pollen allergen Phl p 1 precursor epitope
WGAIWRIDTPDKLTG

SEQ ID NO: 459 - protein, 15 aa, Artificial Sequence
  Phleum pratense Pollen allergen Phl p 11 epitope
RYANPIAFFRKEPLK

SEQ ID NO: 460 - protein, 15 aa, Artificial Sequence
  Phleum pratense Pollen allergen Phl p 2 epitope
EHGSDEWVAMTKGEG

SEQ ID NO: 461 - protein, 15 aa, Artificial Sequence
  Phleum pratense Pollen allergen Phl p 2 epitope
EWVAMTKGEGGVWTF

SEQ ID NO: 462 - protein, 15 aa, Artificial Sequence
  Phleum pratense Pollen allergen Phl p 2 epitope
GVWTFDSEEPLQGPF

SEQ ID NO: 463 - protein, 15 aa, Artificial Sequence
  Phleum pratense Pollen allergen Phl p 2 epitope
KNVFDDVVPEKYTIG

SEQ ID NO: 464 - protein, 15 aa, Artificial Sequence
  Phleum pratense Pollen allergen Phl p 2 epitope
LQGPFNFRFLTEKGM

SEQ ID NO: 465 - protein, 15 aa, Artificial Sequence
  Phleum pratense Pollen allergen Phl p 4 epitope
FKPFAEYKSDYVYEP

SEQ ID NO: 466 - protein, 15 aa, Artificial Sequence
  Phleum pratense Pollen allergen Phl p 4 epitope
FPKEVWEQIFSTWLL

SEQ ID NO: 467 - protein, 15 aa, Artificial Sequence
  Phleum pratense Pollen allergen Phl p 4 epitope
FVHLGHRDNIEDDLL

SEQ ID NO: 468 - protein, 15 aa, Artificial Sequence
  Phleum pratense Pollen allergen Phl p 4 epitope
GIVVAWKVRLLPVPP

SEQ ID NO: 469 - protein, 15 aa, Artificial Sequence
  Phleum pratense Pollen allergen Phl p 4 epitope
NRNNTFKPFAEYKSD

SEQ ID NO: 470 - protein, 12 aa, Artificial Sequence
  Phleum pratense Pollen allergen Phl p 5a epitope
EVKYTVFETALK

SEQ ID NO: 471 - protein, 19 aa, Artificial Sequence
  Phleum pratense Pollen allergen Phl p 5a epitope
NAGFKAALAGAGVQPADKY

SEQ ID NO: 472 - protein, 27 aa, Artificial Sequence
  Phleum pratense Pollen allergen Phl p 5b precursor epitope
AAGKATTEEQKLIEDINVGFKAAVAAA

SEQ ID NO: 473 - protein, 33 aa, Artificial Sequence
  Phleum pratense Pollen allergen Phl p 5b precursor epitope
AAGKATTEEQKLIEDINVGFKAAVAAAASVPAA

SEQ ID NO: 474 - protein, 19 aa, Artificial Sequence
  Phleum pratense Pollen allergen Phl p 5b precursor epitope
AAVAAAASVPAADKFKTFE

SEQ ID NO: 475 - protein, 27 aa, Artificial Sequence
  Phleum pratense Pollen allergen Phl p 5b precursor epitope
AKFDSFVASLTEALRVIAGALEVHAVK

SEQ ID NO: 476 - protein, 19 aa, Artificial Sequence
  Phleum pratense Pollen allergen Phl p 5b precursor epitope
AMSEVQKVSQPATGAATVA

SEQ ID NO: 477 - protein, 20 aa, Artificial Sequence
  Chamaecyparis obtusa Polygalacturonase epitope
ARWKNSKIWLQFAQLTDFNL

SEQ ID NO: 478 - protein, 20 aa, Artificial Sequence
  Chamaecyparis obtusa Polygalacturonase epitope
AVLLVPANKKFFVNNLVFRG

SEQ ID NO: 479 - protein, 20 aa, Artificial Sequence
  Chamaecyparis obtusa Polygalacturonase epitope
DGTIVAQPDPARWKNSKIWL

SEQ ID NO: 480 - protein, 20 aa, Artificial Sequence
  Chamaecyparis obtusa Polygalacturonase epitope
FFVNNLVFRGPCQPHLSFKV

SEQ ID NO: 481 - protein, 20 aa, Artificial Sequence
  Chamaecyparis obtusa Polygalacturonase epitope
FGECEGVKIQGLKIKAPRDS

SEQ ID NO: 482 - protein, 15 aa, Artificial Sequence
  Cryptomeria japonica Polygalacturonase precursor epitope
AAYQNPASWKNNRIW

SEQ ID NO: 483 - protein, 15 aa, Artificial Sequence
  Cryptomeria japonica Polygalacturonase precursor epitope
ACKKPSAMLLVPGNK

SEQ ID NO: 484 - protein, 15 aa, Artificial Sequence
  Cryptomeria japonica Polygalacturonase precursor epitope
AIKFDFSTGLIIQGL

SEQ ID NO: 485 - protein, 15 aa, Artificial Sequence
  Cryptomeria japonica Polygalacturonase precursor epitope
AINIFNVEKYGAVGD

SEQ ID NO: 486 - protein, 15 aa, Artificial Sequence
  Cryptomeria japonica Polygalacturonase precursor epitope
ANGYFSGHVIPACKN

SEQ ID NO: 487 - protein, 16 aa, Artificial Sequence
  Arabidopsis thaliana Probable pectate lyase 18 precursor epitope
GHSDTYSRDKNMQVTI

SEQ ID NO: 488 - protein, 15 aa, Artificial Sequence
  Phleum pratense Profilin-2/4 epitope
LGHDGTVWAQSADFP

SEQ ID NO: 489 - protein, 20 aa, Artificial Sequence
  Hevea brasiliensis Pro-hevein precursor epitope
DEYCSPDHNCQSNCKDSGEG

SEQ ID NO: 490 - protein, 20 aa, Artificial Sequence
  Hevea brasiliensis Pro-hevein precursor epitope
EQCGRQAGGKLCPNNLCCSQ

SEQ ID NO: 491 - protein, 43 aa, Artificial Sequence
  Hevea brasiliensis Pro-hevein precursor epitope
EQCGRQAGGKLCPNNLCCSQWGWCGSTDEYCSPDHNCQSNCKD

SEQ ID NO: 492 - protein, 20 aa, Artificial Sequence
  Hevea brasiliensis Pro-hevein precursor epitope
KLCPNNLCCSQWGWCGSTDE

SEQ ID NO: 493 - protein, 20 aa, Artificial Sequence
  Hevea brasiliensis Pro-hevein precursor epitope
NGGLDLDVNVFRQLDTDGKG

SEQ ID NO: 494 - protein, 10 aa, Artificial Sequence
  Prunus persica pru p 1 epitope
GKCGVSIPYK

SEQ ID NO: 495 - protein, 10 aa, Artificial Sequence
  Prunus persica pru p 1 epitope
ITCGQVSSSL

SEQ ID NO: 496 - protein, 10 aa, Artificial Sequence
  Prunus persica pru p 1 epitope
SIPYKISAST

SEQ ID NO: 497 - protein, 15 aa, Artificial Sequence
  Prunus persica pru p 1 epitope
DRQAACNCLKQLSAS

SEQ ID NO: 498 - protein, 15 aa, Artificial Sequence
  Prunus persica pru p 1 epitope
VNPNNAAALPGKCGV

SEQ ID NO: 499 - protein, 16 aa, Artificial Sequence
  Arabidopsis thaliana Putative pectate lyase 17 precursor epitope
GHNDNFVKDVKMKVTV

SEQ ID NO: 500 - protein, 16 aa, Artificial Sequence
  Homo sapiens RAD51-like 1 isoform 1 epitope
TRLILQYLDSERRQIL

SEQ ID NO: 501 - protein, 16 aa, Artificial Sequence
  Aspergillus fumigatus Ribonuclease mitogillin precursor epitope
DPGPARVIYTYPNKVF

SEQ ID NO: 502 - protein, 20 aa, Artificial Sequence
  Aspergillus fumigatus Ribonuclease mitogillin precursor epitope
ATWTCINQQLNPKTNKWEDK

SEQ ID NO: 503 - protein, 20 aa, Artificial Sequence
  Aspergillus fumigatus Ribonuclease mitogillin precursor epitope
HYLLEFPTFPDGHDYKFDSK

SEQ ID NO: 504 - protein, 20 aa, Artificial Sequence
  Aspergillus fumigatus Ribonuclease mitogillin precursor epitope
KFDSKKPKEDPGPARVIYTY

SEQ ID NO: 505 - protein, 20 aa, Artificial Sequence
  Aspergillus fumigatus Ribonuclease mitogillin precursor epitope
LIKGRTPIKFGKADCDRPPK

SEQ ID NO: 506 - protein, 20 aa, Artificial Sequence
  Aspergillus fumigatus Ribonuclease mitogillin precursor epitope
SYPHWFTNGYDGNGKLIKGR

SEQ ID NO: 507 - protein, 19 aa, Artificial Sequence
  Hevea brasiliensis Rubber elongation factor protein epitope
AEDEDNQQGQGEGLKYLGF

SEQ ID NO: 508 - protein, 19 aa, Artificial Sequence
  Hevea brasiliensis Rubber elongation factor protein epitope
FSNVYLFAKDKSGPLQPGV

SEQ ID NO: 509 - protein, 19 aa, Artificial Sequence
  Hevea brasiliensis Rubber elongation factor protein epitope
KFVDSTVVASVTIIDRSLP

SEQ ID NO: 510 - protein, 19 aa, Artificial Sequence
  Hevea brasiliensis Rubber elongation factor protein epitope
QPGVDIIEGPVKNVAVPLY

SEQ ID NO: 511 - protein, 19 aa, Artificial Sequence
  Hevea brasiliensis Rubber elongation factor protein epitope
RSLPPIVKDASIQVVSAIR

SEQ ID NO: 512 - protein, 17 aa, Artificial Sequence
  Bos taurus Serum albumin precursor epitope
DDSPDLPKLKPDPNTLC

SEQ ID NO: 513 - protein, 20 aa, Artificial Sequence
  Bos taurus Serum albumin precursor epitope
EKDAIPENLPPLTADFAEDK

SEQ ID NO: 514 - protein, 9 aa, Artificial Sequence
  Bos taurus Serum albumin precursor epitope
ESHAGCEKS

SEQ ID NO: 515 - protein, 10 aa, Artificial Sequence
  Bos taurus Serum albumin precursor epitope
HPEYAVSVLL

SEQ ID NO: 516 - protein, 9 aa, Artificial Sequence
  Bos taurus Serum albumin precursor epitope
LSLILNRLC

SEQ ID NO: 517 - protein, 12 aa, Artificial Sequence
  Hevea brasiliensis Small rubber particle protein epitope
DFVRAAGVYAVD

SEQ ID NO: 518 - protein, 12 aa, Artificial Sequence
  Hevea brasiliensis Small rubber particle protein epitope
KYLDFVRAAGVY

SEQ ID NO: 519 - protein, 12 aa, Artificial Sequence
  Hevea brasiliensis Small rubber particle protein epitope
NVVKTVVTPVYY

SEQ ID NO: 520 - protein, 12 aa, Artificial Sequence
  Hevea brasiliensis Small rubber particle protein epitope
PRIVLDVASSVF

SEQ ID NO: 521 - protein, 12 aa, Artificial Sequence
  Hevea brasiliensis Small rubber particle protein epitope
QGYRVSSYLPLL

SEQ ID NO: 522 - protein, 15 aa, Artificial Sequence
  Glycine max Stress-induced protein SAM22 epitope
ALFKAIEAYLLAHPD

SEQ ID NO: 523 - protein, 15 aa, Artificial Sequence
  Cryptomeria japonica Sugi basic protein precursor epitope
AFNVENGNATPQLTK

SEQ ID NO: 524 - protein, 15 aa, Artificial Sequence
  Cryptomeria japonica Sugi basic protein precursor epitope
ANNNYDPWTIYAIGG

SEQ ID NO: 525 - protein, 15 aa, Artificial Sequence
  Cryptomeria japonica Sugi basic protein precursor epitope
AYSDDKSMKVTVAFN

SEQ ID NO: 526 - protein, 15 aa, Artificial Sequence
  Cryptomeria japonica Sugi basic protein precursor epitope
CGQRMPRARYGLVHV

SEQ ID NO: 527 - protein, 15 aa, Artificial Sequence
  Cryptomeria japonica Sugi basic protein precursor epitope
CSNWVWQSTQDVFYN

SEQ ID NO: 528 - protein, 20 aa, Artificial Sequence
  Trichophyton rubrum Tri r 2 allergen epitope
ADFSNYGAVVDVYAPGKDIT

SEQ ID NO: 529 - protein, 20 aa, Artificial Sequence
  Trichophyton rubrum Tri r 2 allergen epitope
AKGVSLVAVKVLDCDGSGSN

SEQ ID NO: 530 - protein, 20 aa, Artificial Sequence
  Trichophyton rubrum Tri r 2 allergen epitope
ASNQAAKAISDAGIFMAVAA

SEQ ID NO: 531 - protein, 20 aa, Artificial Sequence
  Trichophyton rubrum Tri r 2 allergen epitope
DCNGHGTHVAGTVGGTKYGL

SEQ ID NO: 532 - protein, 20 aa, Artificial Sequence
  Trichophyton rubrum Tri r 2 allergen epitope
DPSAGKGVTAYIIDTGIDID

SEQ ID NO: 533 - protein, 12 aa, Artificial Sequence
  Vespula vulgaris Venom allergen 5 precursor epitope
ACKYGSLKPNCG

SEQ ID NO: 534 - protein, 12 aa, Artificial Sequence
  Vespula vulgaris Venom allergen 5 precursor epitope
CNYGPSGNFMNE

SEQ ID NO: 535 - protein, 12 aa, Artificial Sequence
  Vespula vulgaris Venom allergen 5 precursor epitope
DVAKYQVGQNVA

SEQ ID NO: 536 - protein, 12 aa, Artificial Sequence
  Vespula vulgaris Venom allergen 5 precursor epitope
EKWHKHYLVCNY

SEQ ID NO: 537 - protein, 12 aa, Artificial Sequence
  Vespula vulgaris Venom allergen 5 precursor epitope
ELAYVAQVWANQ

SEQ ID NO: 538 - protein, 15 aa, Artificial Sequence
  Corylus avellana 11S globulin-like protein epitope
AFQISREEARRLKYN

SEQ ID NO: 539 - protein, 12 aa, Artificial Sequence
  Carya illinoinensis 11S legumin protein epitope
EESQRQSQQGQR

SEQ ID NO: 540 - protein, 18 aa, Artificial Sequence
  Fagopyrum esculentum 13S globulin epitope
DAHQPTRRVRKGDVVALP

SEQ ID NO: 541 - protein, 12 aa, Artificial Sequence
  Fagopyrum esculentum 13S globulin seed storage protein 1 precursor (Legumin-like protein 1) epitope
FKQNVNRPSRAD

SEQ ID NO: 542 - protein, 12 aa, Artificial Sequence
  Fagopyrum esculentum 13S globulin seed storage protein 3 precursor (Legumin-like protein 3) (Allergen Fag e 1) epitope
DISTKEAFRLKN

SEQ ID NO: 543 - protein, 12 aa, Artificial Sequence
  Anacardium occidentale 2s albumin epitope
CQRQFEEQQRFR

SEQ ID NO: 544 - protein, 10 aa, Artificial Sequence
  Sesamum indicum 2S seed storage protein 1 epitope
HFRECCNEIR

SEQ ID NO: 545 - protein, 10 aa, Artificial Sequence
  Sesamum indicum 2S seed storage protein 1 precursor epitope
CMQWMRSMRG

SEQ ID NO: 546 - protein, 14 aa, Artificial Sequence
  Bertholletia excelsa 2S sulfur-rich seed storage protein precursor (Allergen Ber e 1) epitope
CRCEGLRMMMMRMQ

SEQ ID NO: 547 - protein, 40 aa, Artificial Sequence
  Homo sapiens 52 kDa Ro protein epitope
LEKDEREQLRILGEKEAKLAQQSQALQELISELDRRCHSS

SEQ ID NO: 548 - protein, 10 aa, Artificial Sequence
  Homo sapiens 52-kD SS-A/Ro autoantigen epitope
QEKLQVALGE

SEQ ID NO: 549 - protein, 21 aa, Artificial Sequence
  Homo sapiens 5-hydroxytryptamine (serotonin) receptor 4 epitope
GIIDLIEKRKFNQNSNSTYCV

SEQ ID NO: 550 - protein, 10 aa, Artificial Sequence
  Homo sapiens 60 kDa heat shock protein, mitochondrial precursor epitope
DGVAVLKVGG

SEQ ID NO: 551 - protein, 22 aa, Artificial Sequence
  Homo sapiens 60 kDa SS-A/Ro ribonucleoprotein epitope
ELYKEKALSVETEKLLKYLEAV

SEQ ID NO: 552 - protein, 22 aa, Artificial Sequence
  Homo sapiens 60S acidic ribosomal protein P0 epitope
AKVEAKEESEESDEDMGFGLFD

SEQ ID NO: 553 - protein, 13 aa, Artificial Sequence
  Homo sapiens 60S acidic ribosomal protein P2 epitope
EESDDDMGFGLFD

SEQ ID NO: 554 - protein, 50 aa, Artificial Sequence
  Homo sapiens 64 Kd autoantigen epitope
ATKKEEEKKGGDRNTGLSRDKDKKREEMKEVAKKEDDEKVKGERRNTDTR

SEQ ID NO: 555 - protein, 19 aa, Artificial Sequence
  Homo sapiens 65 kDa heat shock protein epitope
ALLRCIPALDSLTPANEDC

SEQ ID NO: 556 - protein, 14 aa, Artificial Sequence
  Homo sapiens Acetylcholine receptor subunit alpha precursor epitope
AINPESDQPDLSNF

SEQ ID NO: 557 - protein, 21 aa, Artificial Sequence
  Cynodon dactylon acidic Cyn d 1 isoallergen isoform 1 precursor epitope
QDDVIPEDWKPDTVYKSKIQF

SEQ ID NO: 558 - protein, 50 aa, Artificial Sequence
  Cynodon dactylon acidic Cyn d 1 isoallergen isoform 3 precursor epitope
EEDKLRKAGELMLQFRRVKCEYPSDTKITFHVEKGSSPNYLALLVKYAAG

SEQ ID NO: 559 - protein, 8 aa, Artificial Sequence
  Homo sapiens acidic ribosomal phosphoprotein (P0) epitope
AAAAAPAK

SEQ ID NO: 560 - protein, 15 aa, Artificial Sequence
  Homo sapiens acidic ribosomal phosphoprotein (P1) epitope
ESEESDDDMGFGLFD

SEQ ID NO: 561 - protein, 15 aa, Artificial Sequence
  Homo sapiens acidic ribosomal phosphoprotein (P2) epitope
APAAGSAPAAAEEKK

SEQ ID NO: 562 - protein, 16 aa, Artificial Sequence
  Homo sapiens Adrenergic, beta-2-, receptor, surface epitope
HWYRATHQEAINCYAN

SEQ ID NO: 563 - protein, 10 aa, Artificial Sequence
  Homo sapiens Alanyl-tRNA synthetase, cytoplasmic epitope
FIDEPRRRPI

SEQ ID NO: 564 - protein, 7 aa, Artificial Sequence
  Bos taurus albumin epitope
PVESKVT

SEQ ID NO: 565 - protein, 13 aa, Artificial Sequence
  Juglans regia Albumin seed storage protein epitope
GLRGEEMEEMVQS

SEQ ID NO: 566 - protein, 18 aa, Artificial Sequence
  Cochliobolus lunatus alcohol dehydrogenase epitope
AVNGDWPLPTKLPLVGGH

SEQ ID NO: 567 - protein, 37 aa, Artificial Sequence
  Penicillium chrysogenum alkaline serine protease epitope
ANVVQRNAPSWGLSRISSKKSGATDYVYDSTAGEGIV

SEQ ID NO: 568 - protein, 10 aa, Artificial Sequence
  Arachis hypogaea allergen epitope
DDQCQRQLQR

SEQ ID NO: 569 - protein, 15 aa, Artificial Sequence
  Anacardium occidentale allergen Ana o 2 epitope
EESEDEKRRWGQRDN

SEQ ID NO: 570 - protein, 10 aa, Artificial Sequence
  Arachis hypogaea Allergen Ara h 1, clone P41B precursor epitope
AKSSPYQKKT

SEQ ID NO: 571 - protein, 15 aa, Artificial Sequence
  Arachis hypogaea allergen Arah3/Arah4 epitope
AGVALSRLVLRRNAL

SEQ ID NO: 572 - protein, 10 aa, Artificial Sequence
  Arachis hypogaea allergen Arah6 epitope
DRQMVQHFKR

SEQ ID NO: 573 - protein, 11 aa, Artificial Sequence
  Periplaneta americana Allergen Cr-PI epitope
IPKGKKGGQAY

SEQ ID NO: 574 - protein, 8 aa, Artificial Sequence
  Aspergillus fumigatus allergen I/a; Asp f I/a epitope
INQQLNPK

SEQ ID NO: 575 - protein, 10 aa, Artificial Sequence
  Arachis hypogaea Allergen II epitope
DRLQGRQQEQ

SEQ ID NO: 576 - protein, 15 aa, Artificial Sequence
  Lens culinaris allergen Len c 1.0101 epitope
AINASSDLNLIGFGI

SEQ ID NO: 577 - protein, 12 aa, Artificial Sequence
  Dermatophagoides farinae Allergen Mag epitope
DVELSLRSSDIA

SEQ ID NO: 578 - protein, 31 aa, Artificial Sequence
  Penicillium chrysogenum Allergen Pen n 18 epitope
AHIKKSKKGDKKFKGSVANMSLGGGSSRTLD

SEQ ID NO: 579 - protein, 14 aa, Artificial Sequence
  Sinapis alba Allergen Sin a 1 epitope
QGPHVISRIYQTAT

SEQ ID NO: 580 - protein, 12 aa, Artificial Sequence
  Ziziphus mauritiana allergen Ziz m 1 epitope
KTNYSSSIILEY

SEQ ID NO: 581 - protein, 37 aa, Artificial Sequence
  Fagopyrum tataricum allergenic protein epitope
DISTEEAYKLKNGRQEVEVFRPFQSRYEKEEEKERER

SEQ ID NO: 582 - protein, 14 aa, Artificial Sequence
  Homo sapiens alpha 2 interferon epitope
EVVRAEIMRSFSLS

SEQ ID NO: 583 - protein, 30 aa, Artificial Sequence
  Bos taurus alpha S1 casein epitope
EDQAMEDIKQMEAESISSSEEIVPNSVEQK

SEQ ID NO: 584 - protein, 10 aa, Artificial Sequence
  Triticum aestivum Alpha/beta-gliadin A-II precursor epitope
QVSFQQPQQQ

SEQ ID NO: 585 - protein, 10 aa, Artificial Sequence
  Triticum aestivum Alpha/beta-gliadin A-V epitope
LALQTLPAMC

SEQ ID NO: 586 - protein, 10 aa, Artificial Sequence
  Homo sapiens alpha-1 type IV collagen epitope
SRCQVCMRRT

SEQ ID NO: 587 - protein, 12 aa, Artificial Sequence
  Homo sapiens alpha1A-voltage-dependent calcium channel epitope
EDSDEDEFQITE

SEQ ID NO: 588 - protein, 15 aa, Artificial Sequence
  Homo sapiens alpha-2 type XI collagen epitope
GSLDSLRREIEQMRR

SEQ ID NO: 589 - protein, 20 aa, Artificial Sequence
  Bos taurus alpha2(I) collagen epitope
LPGLKGHNGLQGLPGLAGHH

SEQ ID NO: 590 - protein, 5 aa, Artificial Sequence
  Triticum aestivum Alpha-amylase inhibitor 0.28 precursor (CIII) (WMAI-1) epitope
AYPDV

SEQ ID NO: 591 - protein, 17 aa, Artificial Sequence
  Homo sapiens Alpha-enolase epitope
KIHAREIFDSRGNPTVE

SEQ ID NO: 592 - protein, 15 aa, Artificial Sequence
  Homo sapiens alpha-fibrinogen precursor epitope
GPRVVERHQSACKDS

SEQ ID NO: 593 - protein, 12 aa, Artificial Sequence
  Triticum aestivum Alpha-gliadin epitope
LGQGSFRPSQQN

SEQ ID NO: 594 - protein, 10 aa, Artificial Sequence
  Bos taurus Alpha-lactalbumin epitope
KDLKGYGGVS

SEQ ID NO: 595 - protein, 14 aa, Artificial Sequence
  Bos taurus Alpha-lactalbumin precursor epitope
KCEVFRELKDLKGY

SEQ ID NO: 596 - protein, 20 aa, Artificial Sequence
  Bos taurus alpha-S1-casein epitope
LNENLLRFFVAPFPQVFGKE

SEQ ID NO: 597 - protein, 10 aa, Artificial Sequence
  Bos taurus Alpha-S1-casein precursor epitope
AMEDIKQMEA

SEQ ID NO: 598 - protein, 10 aa, Artificial Sequence
  Bos taurus Alpha-S2-casein precursor epitope
ENLCSTFCKE

SEQ ID NO: 599 - protein, 7 aa, Artificial Sequence
  Homo sapiens anti-beta-amyloid peptide immunoglobulin heavy chain variable region epitope
AHIWWND

SEQ ID NO: 600 - protein, 24 aa, Artificial Sequence
  Homo sapiens Aquaporin-4 epitope
FCPDVEFKRRFKEAFSKAAQQTKG

SEQ ID NO: 601 - protein, 15 aa, Artificial Sequence
  Arachis hypogaea Ara h 2.01 allergen epitope
CCNELNEFENNQRCM

SEQ ID NO: 602 - protein, 20 aa, Artificial Sequence
  Homo sapiens ATP-dependent DNA helicase 2 subunit 2 epitope
EEASGSSVTAEEAKKFLAPK

SEQ ID NO: 603 - protein, 28 aa, Artificial Sequence
  Homo sapiens autoantigen epitope
EIRVRLQSASPSTRWTELDDVKRLLKGS

SEQ ID NO: 604 - protein, 16 aa, Artificial Sequence
  Homo sapiens Band 3 anion transport protein epitope
LFKPPKYHPDVPYVKR

SEQ ID NO: 605 - protein, 15 aa, Artificial Sequence
  Glycine max Bd 30K (34 kDa maturing seed protein) epitope
EDWGEDGYIWIQRNT

SEQ ID NO: 606 - protein, 7 aa, Artificial Sequence
  Homo sapiens Bence Jones protein HAG epitope
AWHQQQP

SEQ ID NO: 607 - protein, 8 aa, Artificial Sequence
  Betula pendula Bet v 4 epitope
FARANRGL

SEQ ID NO: 608 - protein, 9 aa, Artificial Sequence
  Musa acuminata beta-1, 3-glucananse epitope
GLFYPNKQP

SEQ ID NO: 609 - protein, 15 aa, Artificial Sequence
  Hevea brasiliensis beta-1,3-glucanase epitope
GLFFPDKRPKYNLNF

SEQ ID NO: 610 - protein, 12 aa, Artificial Sequence
  Olea europaea beta-1,3-glucanase-like protein epitope
AGRNSWNCDFSQ

SEQ ID NO: 611 - protein, 6 aa, Artificial Sequence
  Homo sapiens beta-2-glycoprotein 1 precursor epitope
LKTPRV

SEQ ID NO: 612 - protein, 6 aa, Artificial Sequence
  Homo sapiens beta-2-glycoprotein I epitope
TLRVYK

SEQ ID NO: 613 - protein, 13 aa, Artificial Sequence
  Bos taurus beta-casein epitope
QSKVLPVPQKAVP

SEQ ID NO: 614 - protein, 12 aa, Artificial Sequence
  Bos taurus Beta-casein precursor epitope
DELQDKIHPFAQ

SEQ ID NO: 615 - protein, 10 aa, Artificial Sequence
  Bos taurus Beta-lactoglobulin epitope
AQKKIIAEKT

SEQ ID NO: 616 - protein, 16 aa, Artificial Sequence
  Bos taurus Beta-lactoglobulin precursor epitope
AASDISLLDAQSAPLR

SEQ ID NO: 617 - protein, 7 aa, Artificial Sequence
  Homo sapiens Botulinum neurotoxin type E epitope
WKAPSSP

SEQ ID NO: 618 - protein, 19 aa, Artificial Sequence
  Homo sapiens bullous pemphigoid antigen epitope
KSTAKDCTFKPDFEMTVKE

SEQ ID NO: 619 - protein, 20 aa, Artificial Sequence
  Homo sapiens Bullous pemphigoid antigen 1, isoforms 1/2/3/4/5/8 epitope
LTDTKTGLHFNINEAIEQGT

SEQ ID NO: 620 - protein, 8 aa, Artificial Sequence
  Fagopyrum esculentum BW 16kDa allergen epitope
EGVRDLKE

SEQ ID NO: 621 - protein, 17 aa, Artificial Sequence
  Homo sapiens calcium channel, alpha 1A subunit isoform 3 epitope
GNIGIDVEDEDSDEDEF

SEQ ID NO: 622 - protein, 15 aa, Artificial Sequence
  Homo sapiens Calpastatin epitope
AVCRTSMCSIQSAPP

SEQ ID NO: 623 - protein, 18 aa, Artificial Sequence
  Homo sapiens Calreticulin precursor epitope
KEQFLDGDGWTSRWIESK

SEQ ID NO: 624 - protein, 13 aa, Artificial Sequence
  Homo sapiens Ca-sensing receptor epitope
FVAQNKIDSLNLD

SEQ ID NO: 625 - protein, 9 aa, Artificial Sequence
  Homo sapiens Caspase-8 precursor epitope
DRNGTHLDA

SEQ ID NO: 626 - protein, 14 aa, Artificial Sequence
  Homo sapiens centromere protein A isoform a epitope
GPSRRGPSLGASSH

SEQ ID NO: 627 - protein, 10 aa, Artificial Sequence
  Homo sapiens centromere protein B, 80kDa epitope
MGPKRRQLTF

SEQ ID NO: 628 - protein, 10 aa, Artificial Sequence
  Homo sapiens centromere protein-A epitope
EAPRRRSPSP

SEQ ID NO: 629 - protein, 17 aa, Artificial Sequence
  Betula pendula Chain A, Birch Pollen Profilin epitope
AQSSSFPQFKPQEITGI

SEQ ID NO: 630 - protein, 5 aa, Artificial Sequence
  Homo sapiens Chain A, Crystal Structure Of The Glycosylated Five-Domain Human Beta2-Glycoprotein I Purified From Blood Plasma epitope
RGGMR

SEQ ID NO: 631 - protein, 7 aa, Artificial Sequence
  Homo sapiens Chain H, Three-Dimensional Structure Of A Human Immunoglobulin With A Hinge Deletion epitope
ALPAPIE

SEQ ID NO: 632 - protein, 27 aa, Artificial Sequence
  Homo sapiens cholesterol side-chain cleavage enzyme P450scc (EC 1.14.15.67) epitope
FDPENFDPTRWLSKDKNITYFRNLGFG

SEQ ID NO: 633 - protein, 10 aa, Artificial Sequence
  Homo sapiens citrate synthase epitope
ALKHLPNDPM

SEQ ID NO: 634 - protein, 5 aa, Artificial Sequence
  Homo sapiens claudin 11 epitope
AHRET

SEQ ID NO: 635 - protein, 11 aa, Artificial Sequence
  Homo sapiens Coagulation factor VIII precursor epitope
APDDRSYKSQY

SEQ ID NO: 636 - protein, 20 aa, Artificial Sequence
  Oncorhynchus mykiss collagen a2(I) epitope
MKGLRGHGGLQGMPGPNGPS

SEQ ID NO: 637 - protein, 14 aa, Artificial Sequence
  Homo sapiens Collagen alpha-1(II) chain epitope
ARGAQGPPGATGFP

SEQ ID NO: 638 - protein, 8 aa, Artificial Sequence
  Homo sapiens collagen alpha-1(VII) chain precursor epitope
GTLHVVQR

SEQ ID NO: 639 - protein, 23 aa, Artificial Sequence
  Homo sapiens Collagen alpha-1(XVII) chain epitope
RSILPYGDSMDRIEKDRLQGMAP

SEQ ID NO: 640 - protein, 15 aa, Artificial Sequence
  Homo sapiens Collagen alpha-3(IV) chain epitope
TAIPSCPEGTVPLYS

SEQ ID NO: 641 - protein, 8 aa, Artificial Sequence
  Homo sapiens collagen VII epitope
IIWRSTQG

SEQ ID NO: 642 - protein, 46 aa, Artificial Sequence
  Bos taurus collagen, type I, alpha 2 epitope
APGPDGNNGAQGPPGLQGVQGGKGEQGPAGPPGFQGLPGPAGTAGE

SEQ ID NO: 643 - protein, 9 aa, Artificial Sequence
  Homo sapiens collagen, type II, alpha 1 epitope
PPGPTGASG

SEQ ID NO: 644 - protein, 11 aa, Artificial Sequence
  Homo sapiens collagen, type II, alpha 1 isoform 1 precursor epitope
ARGLTGRPGDA

SEQ ID NO: 645 - protein, 11 aa, Artificial Sequence
  Homo sapiens collagen, type II, alpha 1 isoform 2 precursor epitope
LVGPRGERGFP

SEQ ID NO: 646 - protein, 9 aa, Artificial Sequence
  Homo sapiens Complement C1q subcomponent subunit A epitope
KGEQGEPGA

SEQ ID NO: 647 - protein, 10 aa, Artificial Sequence
  Homo sapiens Condensin-2 complex subunit D3 epitope
PTPETGPLQR

SEQ ID NO: 648 - protein, 15 aa, Artificial Sequence
  Arachis hypogaea Conglutin-7 precursor epitope
AAHASARQQWELQGD

SEQ ID NO: 649 - protein, 8 aa, Artificial Sequence
  Periplaneta americana Cr-PII allergen epitope
IRSWFGLP

SEQ ID NO: 650 - protein, 11 aa, Artificial Sequence
  Cochliobolus lunatus Cytochrome c epitope
ENPKKYIPGTK

SEQ ID NO: 651 - protein, 10 aa, Artificial Sequence
  Rattus norvegicus Cytochrome P450 3A1 epitope
DMVLNETLRL

SEQ ID NO: 652 - protein, 15 aa, Artificial Sequence
  Homo sapiens cytoskeleton-associated protein 5 isoform b epitope
CQALVRMLAKKPGWK

SEQ ID NO: 653 - protein, 9 aa, Artificial Sequence
  Dermatophagoides farinae Der f 2 epitope
IATHAKIRD

SEQ ID NO: 654 - protein, 15 aa, Artificial Sequence
  Dermatophagoides farinae Der f 7 allergen epitope
HIGGLSILDPIFGVL

SEQ ID NO: 655 - protein, 43 aa, Artificial Sequence
  Dermatophagoides pteronyssinus Der p 1 allergen epitope
AREQSCRRPNAQRFGISNYCQIYPPNANKIREALAQTHSAIAV

SEQ ID NO: 656 - protein, 15 aa, Artificial Sequence
  Dermatophagoides pteronyssinus Der p 7 allergen polypeptide epitope
HIGGLSILDPIFAVL

SEQ ID NO: 657 - protein, 12 aa, Artificial Sequence
  Homo sapiens Desmoglein-1 epitope
REWIKFAAACRE

SEQ ID NO: 658 - protein, 12 aa, Artificial Sequence
  Homo sapiens Desmoglein-3 precursor epitope
REWVKFAKPCRE

SEQ ID NO: 659 - protein, 30 aa, Artificial Sequence
  Homo sapiens desmoglein-3 preproprotein epitope
SQEPAGTPMFLLSRNTGEVRTLTNSLDREQ

SEQ ID NO: 660 - protein, 12 aa, Artificial Sequence
  Homo sapiens desmoplakin epitope
GNSSYSYSYSFS

SEQ ID NO: 661 - protein, 20 aa, Artificial Sequence
  Homo sapiens desmoplakin isoform II epitope
LVDRKTGSQYDIQDAIDKGL

SEQ ID NO: 662 - protein, 18 aa, Artificial Sequence
  Homo sapiens dihydrolipoamide S-acetyltransferase (E2 component of pyruvate dehydrogenase complex), isoform CRA_a epitope
AEIETDKATIGFEVQEEG

SEQ ID NO: 663 - protein, 17 aa, Artificial Sequence
  Homo sapiens DNA topoisomerase 1 epitope
GVPIEKIYNKTQREKFA

SEQ ID NO: 664 - protein, 20 aa, Artificial Sequence
  Homo sapiens DNA topoisomerase I epitope
ELDGQEYVVEFDFLGKDSIR

SEQ ID NO: 665 - protein, 20 aa, Artificial Sequence
  Homo sapiens DNA topoisomerase II beta epitope
HPMLPNYKNFKGTIQELGQN

SEQ ID NO: 666 - protein, 7 aa, Artificial Sequence
  Homo sapiens DNA-directed RNA polymerase II subunit RPB1 epitope
YSPTSPS

SEQ ID NO: 667 - protein, 36 aa, Artificial Sequence
  Homo sapiens E3 ubiquitin-protein ligase TRIM9 isoform 2 epitope
AFNKTGVSPYSKTLVLQTSEGKALQQYPSERELRGI

SEQ ID NO: 668 - protein, 39 aa, Artificial Sequence
  Candida albicans Enolase 1 (2-phosphoglycerate dehydratase) (2-phospho-D-glycerate hydro-lyase) epitope
QAANDSYAAGWGVMVSHRSGETEDTFIADLSVGLRSGQI

SEQ ID NO: 669 - protein, 16 aa, Artificial Sequence
  Homo sapiens enolase 1 variant epitope
KIHAREIFDSRGNPTV

SEQ ID NO: 670 - protein, 21 aa, Artificial Sequence
  Hevea brasiliensis ENSP-like protein epitope
FPLITCCGYGGKYNFSVTAPC

SEQ ID NO: 671 - protein, 20 aa, Artificial Sequence
  Homo sapiens envoplakin epitope
AGETKPSSSLSIGSIISKSP

SEQ ID NO: 672 - protein, 12 aa, Artificial Sequence
  Fagopyrum esculentum Fag e 1 epitope
AVVLKAGNEGLE

SEQ ID NO: 673 - protein, 3 aa, Artificial Sequence
  Homo sapiens Fas AMA epitope
CVP

SEQ ID NO: 674 - protein, 18 aa, Artificial Sequence
  Homo sapiens FGA protein epitope
SRALAREVDLKDYEDQQK

SEQ ID NO: 675 - protein, 20 aa, Artificial Sequence
  Homo sapiens FGB protein epitope
ARGHRPLDKKREEAPSLRPA

SEQ ID NO: 676 - protein, 19 aa, Artificial Sequence
  Homo sapiens fibrin beta epitope
ANKYQISVNKYRGTAGNAL

SEQ ID NO: 677 - protein, 15 aa, Artificial Sequence
  Homo sapiens fibrinogen alpha chain isoform alpha preproprotein epitope
DSPGSGNARPNNPDW

SEQ ID NO: 678 - protein, 17 aa, Artificial Sequence
  Homo sapiens Fibrinogen alpha chain precursor epitope
FLAEGGGVRGPRVVERH

SEQ ID NO: 679 - protein, 20 aa, Artificial Sequence
  Homo sapiens fibrinogen alpha chain preproprotein, isoform alpha epitope
DHEGTHSTKRGHAKSRPVRG

SEQ ID NO: 680 - protein, 15 aa, Artificial Sequence
  Homo sapiens fibrinogen beta chain epitope
PRKQCSKEDGGGWWY

SEQ ID NO: 681 - protein, 17 aa, Artificial Sequence
  Homo sapiens fibrinogen beta chain, isoform CRA_d epitope
NEEGFFSARGHRPLDKK

SEQ ID NO: 682 - protein, 24 aa, Artificial Sequence
  Homo sapiens fibrinogen beta chain, isoform CRA_i epitope
EEAPSLRPAPPPISGGGYRARPAK

SEQ ID NO: 683 - protein, 6 aa, Artificial Sequence
  Homo sapiens Fibronectin precursor epitope
LTSRPA

SEQ ID NO: 684 - protein, 18 aa, Artificial Sequence
  Homo sapiens filaggrin epitope
DSGHRGYSGSQASDNEGH

SEQ ID NO: 685 - protein, 8 aa, Artificial Sequence
  Homo sapiens Follistatin-related protein 1 epitope
LKFVEQNE

SEQ ID NO: 686 - protein, 10 aa, Artificial Sequence
  Homo sapiens Forkhead box protein E3 epitope
PTPAPGPGRR

SEQ ID NO: 687 - protein, 7 aa, Artificial Sequence
  Homo sapiens GAD65 autoantigen glutamic acid decarboxylase epitope
APAMIPP

SEQ ID NO: 688 - protein, 10 aa, Artificial Sequence
  Triticum aestivum Gamma-gliadin precursor epitope
LQPQQPFPQQ

SEQ ID NO: 689 - protein, 21 aa, Artificial Sequence
  Chironomus thummi thummi Globin CTT-III epitope
AHTDFAGAEAAWGATLDTFFG

SEQ ID NO: 690 - protein, 11 aa, Artificial Sequence
  Chironomus thummi thummi Globin CTT-III precursor epitope
GVTHDQLNNFR

SEQ ID NO: 691 - protein, 23 aa, Artificial Sequence
  Chironomus thummi thummi Globin CTT-IV precursor epitope
KAHTDFAGAEAAWGATLDAFFGM

SEQ ID NO: 692 - protein, 35 aa, Artificial Sequence
  Chironomus thummi thummi Globin CTT-VI precursor epitope
IVSFLSEVISLAGSDANIPAIQNLAKELATSHKPR

SEQ ID NO: 693 - protein, 35 aa, Artificial Sequence
  Chironomus thummi thummi Globin CTT-VIII epitope
IVGFFSEVIGLIGNPENRPALKTLIDGLASSHKAR

SEQ ID NO: 694 - protein, 9 aa, Artificial Sequence
  Hevea brasiliensis Glucan endo-1,3-beta-glucosidase, basic vacuolar isoform epitope
AWLAQFVLP

SEQ ID NO: 695 - protein, 20 aa, Artificial Sequence
  Homo sapiens glutamate decarboxylase epitope
FRERQSSKNLLSCENSDRDA

SEQ ID NO: 696 - protein, 20 aa, Artificial Sequence
  Homo sapiens Glutamate decarboxylase 1 epitope
MASSTPSSSATSSNAGADPN

SEQ ID NO: 697 - protein, 19 aa, Artificial Sequence
  Homo sapiens Glutamate decarboxylase 2 epitope
PGSGFWSFGSEDGSGDSEN

SEQ ID NO: 698 - protein, 15 aa, Artificial Sequence
  Homo sapiens glutamate receptor, ionotropic, N-methyl D-aspartate 2A epitope
SVSYDDWDYSLEARV

SEQ ID NO: 699 - protein, 14 aa, Artificial Sequence
  Homo sapiens glutathione peroxidase-GI epitope
NEHPVFAYLKDKLP

SEQ ID NO: 700 - protein, 14 aa, Artificial Sequence
  Triticum aestivum Glutenin, high molecular weight subunit DX5 epitope
AQGQQPGQGQQGQQ

SEQ ID NO: 701 - protein, 5 aa, Artificial Sequence
  Triticum aestivum Glutenin, high molecular weight subunit DX5 precursor epitope
QQPGQ

SEQ ID NO: 702 - protein, 5 aa, Artificial Sequence
  Triticum aestivum Glutenin, low molecular weight subunit precursor epitope
QQQPP

SEQ ID NO: 703 - protein, 15 aa, Artificial Sequence
  Phaseolus vulgaris Glycine-rich cell wall structural protein 1.8 precursor epitope
GGYGDGGAHGGGYGG

SEQ ID NO: 704 - protein, 15 aa, Artificial Sequence
  Arachis hypogaea Glycinin epitope
ALSRLVLRRNALRRP

SEQ ID NO: 705 - protein, 13 aa, Artificial Sequence
  Glycine max Glycinin G1 precursor epitope
GAIVTVKGGLSVI

SEQ ID NO: 706 - protein, 15 aa, Artificial Sequence
  Glycine max Glycinin G2 precursor epitope
ALSRCTLNRNALRRP

SEQ ID NO: 707 - protein, 15 aa, Artificial Sequence
  Holcus lanatus group V allergen epitope
ANVPPADKYKTFEAA

SEQ ID NO: 708 - protein, 38 aa, Artificial Sequence
  Homo sapiens Gu protein epitope
IDAPKPKKMKKEKEMNGETREKSPKLKNGFPHPEPDCN

SEQ ID NO: 709 - protein, 17 aa, Artificial Sequence
  Homo sapiens H1 histone family, member 0 epitope
KEIKKVATPKKASKPKK

SEQ ID NO: 710 - protein, 12 aa, Artificial Sequence
  Homo sapiens heat shock 60kDa protein 1 (chaperonin) epitope
AYAKDVKFGADA

SEQ ID NO: 711 - protein, 6 aa, Artificial Sequence
  Homo sapiens Heat shock protein HSP 90-beta epitope
GLELPE

SEQ ID NO: 712 - protein, 15 aa, Artificial Sequence
  Homo sapiens high mobility group protein 17 epitope
KKAPAKKGEKVPKGK

SEQ ID NO: 713 - protein, 22 aa, Artificial Sequence
  Homo sapiens High mobility group protein B1 epitope
AKGKPDAAKKGVVKAEKSKKKK

SEQ ID NO: 714 - protein, 15 aa, Artificial Sequence
  Homo sapiens high-mobility group box 2 epitope
FEDMAKSDKARYDRE

SEQ ID NO: 715 - protein, 43 aa, Artificial Sequence
  Homo sapiens histidyl-tRNA synthetase, cytoplasmic epitope
AERAALEELVKLQGERVRGLKQQKASAELIEEEVAKLLKLKAQ

SEQ ID NO: 716 - protein, 16 aa, Artificial Sequence
  Homo sapiens Histone H1.4 epitope
SETAPAAPAAPAPAEK

SEQ ID NO: 717 - protein, 15 aa, Artificial Sequence
  Homo sapiens histone H1b epitope
KPKAAKPKKAAAKKK

SEQ ID NO: 718 - protein, 20 aa, Artificial Sequence
  Homo sapiens Histone H2A.Z epitope
GKAKTKAVSRSQRAGLQFPV

SEQ ID NO: 719 - protein, 15 aa, Artificial Sequence
  Homo sapiens histone H3 epitope
LPFQRLVREIAQDFK

SEQ ID NO: 720 - protein, 10 aa, Artificial Sequence
  Homo sapiens Histone H3-like centromeric protein A epitope
KPEAPRRRSP

SEQ ID NO: 721 - protein, 8 aa, Artificial Sequence
  Homo sapiens HLA class I histocompatibility antigen, B-27 alpha chain precursor epitope
KAKAQTDR

SEQ ID NO: 722 - protein, 16 aa, Artificial Sequence
  Homo sapiens HLA-B27 epitope
AKAQTDREDLRTLLRY

SEQ ID NO: 723 - protein, 20 aa, Artificial Sequence
  Homo sapiens HLA-DR3 epitope
RPDAEYWNSQKDLLEQKRGR

SEQ ID NO: 724 - protein, 15 aa, Artificial Sequence
  Homo sapiens HMG-17 epitope
DGKAKVKDEPQRRSA

SEQ ID NO: 725 - protein, 13 aa, Artificial Sequence
  Homo sapiens HNRNPA2B1 protein epitope
ETTEESLRNYYEQ

SEQ ID NO: 726 - protein, 35 aa, Artificial Sequence
  Homo sapiens hypothetical protein epitope
ANEDAAQGIANWDAVQDIANEDGFHGIDIEDAAQG

SEQ ID NO: 727 - protein, 12 aa, Artificial Sequence
  Oryza sativa Japonica Group hypothetical protein epitope
AFNHFGIQLVQR

SEQ ID NO: 728 - protein, 20 aa, Artificial Sequence
  Homo sapiens Ig alpha-1 chain C region epitope
PVPSTPPTPSPSTPPTPSPS

SEQ ID NO: 729 - protein, 7 aa, Artificial Sequence
  Homo sapiens Ig gamma-1 chain C region epitope
KFNWYVD

SEQ ID NO: 730 - protein, 7 aa, Artificial Sequence
  Homo sapiens Ig gamma-3 chain C region epitope
DGSFFLY

SEQ ID NO: 731 - protein, 7 aa, Artificial Sequence
  Homo sapiens Ig heavy chain V-III region (ART) epitope
CSVMHEG

SEQ ID NO: 732 - protein, 16 aa, Artificial Sequence
  Homo sapiens Ig lambda chain V-II region MGC epitope
SGLQAEDEADYYCSSY

SEQ ID NO: 733 - protein, 7 aa, Artificial Sequence
  Homo sapiens Ig L-chain V-region epitope
APSVTLF

SEQ ID NO: 734 - protein, 7 aa, Artificial Sequence
  Homo sapiens Immunoglobulin heavy chain epitope
DKSRWQE

SEQ ID NO: 735 - protein, 16 aa, Artificial Sequence
  Homo sapiens immunoglobulin light chain epitope
KATLVCLISDFYPGAV

SEQ ID NO: 736 - protein, 7 aa, Artificial Sequence
  Homo sapiens immunoglobulin light chain variable region epitope
AGEKVTM

SEQ ID NO: 737 - protein, 3 aa, Artificial Sequence
  Homo sapiens Insulin precursor epitope
TSI

SEQ ID NO: 738 - protein, 14 aa, Artificial Sequence
  Homo sapiens Integrin alpha-6 epitope
LKRDMKSAHLLPEH

SEQ ID NO: 739 - protein, 18 aa, Artificial Sequence
  Homo sapiens Integrin beta-3 precursor epitope
CAPESIEFPVSEARVLED

SEQ ID NO: 740 - protein, 14 aa, Artificial Sequence
  Homo sapiens interferon alpha 2 epitope
CDLPQTHSLGSRRT

SEQ ID NO: 741 - protein, 14 aa, Artificial Sequence
  Homo sapiens interferon alpha A epitope
EDSILAVRKYFQRI

SEQ ID NO: 742 - protein, 12 aa, Artificial Sequence
  Homo sapiens interferon beta precursor epitope
HLKRYYGRILHY

SEQ ID NO: 743 - protein, 14 aa, Artificial Sequence
  Homo sapiens interferon-alpha 2 epitope
LMLLAQMRRISLFS

SEQ ID NO: 744 - protein, 37 aa, Artificial Sequence
  Homo sapiens Islet amyloid polypeptide precursor epitope
MGILKLQVFLIVLSVALNHLKATPIESHQVEKRKCNT

SEQ ID NO: 745 - protein, 14 aa, Artificial Sequence
  Bos taurus Kappa-casein precursor epitope
AKYIPIQYVLSRYP

SEQ ID NO: 746 - protein, 10 aa, Artificial Sequence
  Homo sapiens Ku antigen epitope
RGDGPFRLGG

SEQ ID NO: 747 - protein, 15 aa, Artificial Sequence
  Homo sapiens leukotriene B4 receptor 2 epitope
GRGNGDPGGGMEKDG

SEQ ID NO: 748 - protein, 14 aa, Artificial Sequence
  Homo sapiens liver histone H1e epitope
IKKVATPKKASPKK

SEQ ID NO: 749 - protein, 23 aa, Artificial Sequence
  Homo sapiens Lupus La protein epitope
AQPGSGKGKVQFQGKKTKFASDD

SEQ ID NO: 750 - protein, 30 aa, Artificial Sequence
  Homo sapiens lymphocyte activation gene 3 protein precursor epitope
GPPAAAPGHPLAPGPHPAAPSSWGPRPRRY

SEQ ID NO: 751 - protein, 10 aa, Artificial Sequence
  Homo sapiens m3 muscarinic cholinergic receptor epitope
EPTITFGTAI

SEQ ID NO: 752 - protein, 20 aa, Artificial Sequence
  Alternaria alternata Major allergen Alt a 1 precursor epitope
ADPVTTEGDYVVKISEFYGR

SEQ ID NO: 753 - protein, 15 aa, Artificial Sequence
  Anisakis simplex Major allergen Ani s 1 epitope
CKMPDRGACALGKKP

SEQ ID NO: 754 - protein, 13 aa, Artificial Sequence
  Aspergillus fumigatus Major allergen Asp f 1 epitope
LNPKTNKWEDKRY

SEQ ID NO: 755 - protein, 10 aa, Artificial Sequence
  Aspergillus fumigatus Major allergen Asp f 2 epitope
AHILRWGNES

SEQ ID NO: 756 - protein, 20 aa, Artificial Sequence
  Bos taurus major allergen beta-lactoglobulin epitope
LQKWENDECAQKKIIAEKTK

SEQ ID NO: 757 - protein, 14 aa, Artificial Sequence
  Felis catus Major allergen I polypeptide chain 1 precursor epitope
DAKMTEEDKENALS

SEQ ID NO: 758 - protein, 14 aa, Artificial Sequence
  Felis catus Major allergen I polypeptide chain 2 precursor epitope
EPERTAMKKIQDCY

SEQ ID NO: 759 - protein, 11 aa, Artificial Sequence
  Felis catus major allergen I, polypeptide chain 1 epitope
LLDKIYTSPLC

SEQ ID NO: 760 - protein, 29 aa, Artificial Sequence
  Turbo cornutus major allergen Tur c1 - Turbo cornutus epitope
LEDELLAEKEKYKAISDELDQTFAELAGY

SEQ ID NO: 761 - protein, 25 aa, Artificial Sequence
  Dermatophagoides pteronyssinus major house dust allergen epitope
LAHRNQSLDLAEQELVDCASQHGCH

SEQ ID NO: 762 - protein, 9 aa, Artificial Sequence
  Hevea brasiliensis Major latex allergen Hev b 5 epitope
APPASEQET

SEQ ID NO: 763 - protein, 43 aa, Artificial Sequence
  Dermatophagoides pteronyssinus Major mite fecal allergen Der p 1 epitope
AREQSCRRPNAQRFGISNYCQIYPPNANKIREALAQPQRYCRH

SEQ ID NO: 764 - protein, 12 aa, Artificial Sequence
  Olea europaea Major pollen allergen epitope
FTEVGYTRAEGL

SEQ ID NO: 765 - protein, 10 aa, Artificial Sequence
  Betula pendula Major pollen allergen Bet v 1-A epitope
DGDNLFPKVA

SEQ ID NO: 766 - protein, 11 aa, Artificial Sequence
  Chamaecyparis obtusa Major pollen allergen Cha o 1 precursor epitope
WRSTQDSFNNG

SEQ ID NO: 767 - protein, 17 aa, Artificial Sequence
  Corylus avellana Major pollen allergen Cor a 1 epitope
YVLDGDKLLPKVAPQAL

SEQ ID NO: 768 - protein, 27 aa, Artificial Sequence
  Holcus lanatus Major pollen allergen Hol l 1 precursor epitope
AKSTWYGKPTGAGPKDNGGACGYKDVD

SEQ ID NO: 769 - protein, 12 aa, Artificial Sequence
  Juniperus ashei Major pollen allergen Jun a 1 precursor epitope
AFNQFGPNAGQR

SEQ ID NO: 770 - protein, 34 aa, Artificial Sequence
  Olea europaea major pollen allergen Ole e 1 epitope
SGRKDCNEIPTEGWVKPSLKFILNTVNGTTRTVN

SEQ ID NO: 771 - protein, 9 aa, Artificial Sequence
  Malus x domestica mal d 3 epitope
RTTADRQTA

SEQ ID NO: 772 - protein, 15 aa, Artificial Sequence
  Homo sapiens MBP protein epitope
ENPVVHFFKNIVTPR

SEQ ID NO: 773 - protein, 30 aa, Artificial Sequence
  Homo sapiens melanin-concentrating hormone receptor 1, isoform CRA_a epitope
AEHASRMSVLRAKPMSNSQRLLLLSPGSPP

SEQ ID NO: 774 - protein, 16 aa, Artificial Sequence
  Homo sapiens Melanocyte protein Pmel 17 precursor epitope
QVPTTEVVGTTPGQAP

SEQ ID NO: 775 - protein, 6 aa, Artificial Sequence
  Homo sapiens MHC classII HLA-DRB1 epitope
EQRRAA

SEQ ID NO: 776 - protein, 20 aa, Artificial Sequence
  Homo sapiens MHC HLA-DR1-beta epitope
RPDAEYWNSQKDLLEQRRAA

SEQ ID NO: 777 - protein, 16 aa, Artificial Sequence
  Blomia tropicalis Mite allergen Blo t 5 epitope
EEAQTLSKILLKDLKE

SEQ ID NO: 778 - protein, 5 aa, Artificial Sequence
  Dermatophagoides farinae Mite group 2 allergen Der f 2 precursor epitope
DPCII

SEQ ID NO: 779 - protein, 15 aa, Artificial Sequence
  Dermatophagoides pteronyssinus Mite group 2 allergen Der p 2 precursor epitope
DQVDVKDCANHEIKK

SEQ ID NO: 780 - protein, 33 aa, Artificial Sequence
  Lepidoglyphus destructor Mite group 2 allergen Lep d 2 precursor epitope
AANQDTAKVTIKVLAKVAGTTIQVPGLETDGCK

SEQ ID NO: 781 - protein, 6 aa, Artificial Sequence
  Triticum aestivum monomeric alpha-amylase inhibitor epitope
AASVPE

SEQ ID NO: 782 - protein, 21 aa, Artificial Sequence
  Homo sapiens Muscarinic acetylcholine receptor M1 epitope
QYLVGERTVLAGQCYIQFLSQ

SEQ ID NO: 783 - protein, 17 aa, Artificial Sequence
  Homo sapiens myelin associated glycoprotein epitope
DSYTLTEELAYAEIRVK

SEQ ID NO: 784 - protein, 13 aa, Artificial Sequence
  Homo sapiens Myelin basic protein epitope
IVTPRTPPPSQGK

SEQ ID NO: 785 - protein, 26 aa, Artificial Sequence
  Homo sapiens myelin oligodendrocyte glycoprotein epitope
ALVGDEVELPCRISPGKNATGMELGW

SEQ ID NO: 786 - protein, 5 aa, Artificial Sequence
  Homo sapiens myelin oligodendrocyte glycoprotein isoform alpha6 precursor epitope
HRTFE

SEQ ID NO: 787 - protein, 5 aa, Artificial Sequence
  Homo sapiens myelin proteolipid protein epitope
ADARM

SEQ ID NO: 788 - protein, 21 aa, Artificial Sequence
  Homo sapiens Myelin-associated glycoprotein precursor epitope
GHWGAWMPSSISAFEGTCVSI

SEQ ID NO: 789 - protein, 26 aa, Artificial Sequence
  Homo sapiens Myelin-oligodendrocyte glycoprotein precursor epitope
GQFRVIGPRHPIRALVGDEVELPCRI

SEQ ID NO: 790 - protein, 8 aa, Artificial Sequence
  Homo sapiens Myeloblastin precursor epitope
AHRPPSPA

SEQ ID NO: 791 - protein, 10 aa, Artificial Sequence
  Homo sapiens Myeloperoxidase epitope
GSASPMELLS

SEQ ID NO: 792 - protein, 20 aa, Artificial Sequence
  Homo sapiens Myosin-11 epitope
ALKTELEDTLDSTATQQELR

SEQ ID NO: 793 - protein, 8 aa, Artificial Sequence
  Homo sapiens Neurofilament heavy polypeptide (NF-H) (Neurofilament triplet H protein) (200 kDa neurofilament protein) epitope
AKSPEKAK

SEQ ID NO: 794 - protein, 16 aa, Artificial Sequence
  Homo sapiens nicotinic acetylcholine receptor alpha subunit|AChR alpha subunit epitope
EVNQIVTTNVRLKQQW

SEQ ID NO: 795 - protein, 10 aa, Artificial Sequence
  Homo sapiens Non-histone chromosomal protein HMG-17 epitope
VKDEPQRRSA

SEQ ID NO: 796 - protein, 9 aa, Artificial Sequence
  Prunus armeniaca Non-specific lipid-transfer protein 1 epitope
VNPNNAAAL

SEQ ID NO: 797 - protein, 15 aa, Artificial Sequence
  Prunus armeniaca Non-specific lipid-transfer protein 1 (LTP 1) (Major allergen Pru ar 3) epitope
LARTTPDRRTACNCL

SEQ ID NO: 798 - protein, 18 aa, Artificial Sequence
  Prunus domestica Non-specific lipid-transfer protein 1 (LTP 1) (Major allergen Pru d 3) epitope
LARTTADRRAACNCLKQL

SEQ ID NO: 799 - protein, 15 aa, Artificial Sequence
  Malus x domestica Non-specific lipid-transfer protein precursor (LTP) (Allergen Mal d 3) epitope
ADRQTACNCLKNLAG

SEQ ID NO: 800 - protein, 10 aa, Artificial Sequence
  Homo sapiens NR2 subunit NMDA receptor epitope
DWEYSVWLSN

SEQ ID NO: 801 - protein, 8 aa, Artificial Sequence
  Homo sapiens nuclear autoantigen Sp-100 isoform 1 epitope
EVFISAPR

SEQ ID NO: 802 - protein, 12 aa, Artificial Sequence
  Olea europaea Ole e 1 protein epitope
EDVPQPPVSQFH

SEQ ID NO: 803 - protein, 25 aa, Artificial Sequence
  Olea europaea Ole e 1.0102 protein epitope
EDVPQPPVSQFHIQGQVYCDTCRAG

SEQ ID NO: 804 - protein, 10 aa, Artificial Sequence
  Triticum aestivum Omega gliadin storage protein epitope
QQPQQSFPQQ

SEQ ID NO: 805 - protein, 7 aa, Artificial Sequence
  Triticum aestivum omega-5 gliadin epitope
QQFHQQQ

SEQ ID NO: 806 - protein, 35 aa, Artificial Sequence
  Aspergillus fumigatus Oryzin precursor epitope
ASNTSPASAPNALTVAAINKSNARASFSNYGSVVD

SEQ ID NO: 807 - protein, 9 aa, Artificial Sequence
  Gallus gallus Ovalbumin epitope
CFDVFKELK

SEQ ID NO: 808 - protein, 10 aa, Artificial Sequence
  Gallus gallus Ovomucoid epitope
CNFCNAVVES

SEQ ID NO: 809 - protein, 14 aa, Artificial Sequence
  Gallus gallus Ovomucoid precursor epitope
AEVDCSRFPNATDK

SEQ ID NO: 810 - protein, 10 aa, Artificial Sequence
  Glycine max P34 probable thiol protease precursor epitope
ASWDWRKKGV

SEQ ID NO: 811 - protein, 10 aa, Artificial Sequence
  Glycine max P34 probable thiol protease precursor; Gly m 1 epitope
PQEFSKKTYQ

SEQ ID NO: 812 - protein, 9 aa, Artificial Sequence
  Homo sapiens p70 autoantigen epitope
EALTKHFQD

SEQ ID NO: 813 - protein, 20 aa, Artificial Sequence
  Homo sapiens PADI-H protein epitope
KAASGSTGDQKVQISYYGPK

SEQ ID NO: 814 - protein, 9 aa, Artificial Sequence
  Parietaria judaica Par j epitope
GTSSCRLVP

SEQ ID NO: 815 - protein, 47 aa, Artificial Sequence
  Blomia tropicalis Paramyosin epitope
EKLRDQKEALARENKKLADDLAEAKSQLNDAHRRIHEQEIEIKRLEN

SEQ ID NO: 816 - protein, 8 aa, Artificial Sequence
  Gadus morhua callarias Parvalbumin beta epitope
AAEAACFK

SEQ ID NO: 817 - protein, 15 aa, Artificial Sequence
  Salmo salar parvalbumin like 1 epitope
ADIKTALEARKAADT

SEQ ID NO: 818 - protein, 12 aa, Artificial Sequence
  Juniperus ashei Pathogenesis-related protein precursor epitope
ADINAVCPSELK

SEQ ID NO: 819 - protein, 12 aa, Artificial Sequence
  Nicotiana tabacum Pectate lyase epitope
AYNHFGKRLDQR

SEQ ID NO: 820 - protein, 12 aa, Artificial Sequence
  Musa acuminata AAA Group pectate lyase 2 epitope
AFNHFGEGLIQR

SEQ ID NO: 821 - protein, 15 aa, Artificial Sequence
  Farfantepenaeus aztecus Pen a 1 allergen epitope
ANIQLVEKDKALSNA

SEQ ID NO: 822 - protein, 43 aa, Artificial Sequence
  Dermatophagoides pteronyssinus Peptidase 1 precursor (Major mite fecal allergen Der p 1) (Allergen Der p I) epitope
AREQSCRRPNAQRFGISNYCQIYPPNVNKIREALAQTHSAIAV

SEQ ID NO: 823 - protein, 3 aa, Artificial Sequence
  Homo sapiens pericentriolar material 1 protein epitope
KDC

SEQ ID NO: 824 - protein, 20 aa, Artificial Sequence
  Homo sapiens Periplakin epitope
IHDRKSGKKFSIEEALQSGR

SEQ ID NO: 825 - protein, 45 aa, Artificial Sequence
  Apis mellifera Phospholipase A2 precursor epitope
LIDTKCYKLEHPVTGCGERTEGRCLHYTVDKSKPKVYQWFDLRKY

SEQ ID NO: 826 - protein, 14 aa, Artificial Sequence
  Myrmecia pilosula Pilosulin-1 precursor (Major allergen Myr p 1) (Myr p I) epitope
KEAIPMAVEMAKSQ

SEQ ID NO: 827 - protein, 8 aa, Artificial Sequence
  Homo sapiens plasma protease C1 inhibitor precursor epitope
ASAISVAR

SEQ ID NO: 828 - protein, 6 aa, Artificial Sequence
  Homo sapiens platelet glycoprotein IIIa epitope
RARAKW

SEQ ID NO: 829 - protein, 12 aa, Artificial Sequence
  Homo sapiens plexin domain containing 1, isoform CRA_b epitope
NCSWCHVLQRCS

SEQ ID NO: 830 - protein, 15 aa, Artificial Sequence
  Homo sapiens PM/Scl 100kD nucleolar protein epitope
CIAAKKIKQSVGNKS

SEQ ID NO: 831 - protein, 8 aa, Artificial Sequence
  Betula pendula Polcalcin Bet v 4 epitope
FGRANRGL

SEQ ID NO: 832 - protein, 36 aa, Artificial Sequence
  Phleum pratense Polcalcin Phl p 7 (Calcium-binding pollen allergen Phl p 7) (P7) epitope
ADDMERIFKRFDTNGDGKISLSELTDALRTLGSTSA

SEQ ID NO: 833 - protein, 25 aa, Artificial Sequence
  Lolium perenne pollen allergen epitope
EGGTKSEVEDVIPEGWKADTSYSAK

SEQ ID NO: 834 - protein, 12 aa, Artificial Sequence
  Ambrosia artemisiifolia Pollen allergen Amb a 1.4 epitope
AFNKFTDNVDQR

SEQ ID NO: 835 - protein, 8 aa, Artificial Sequence
  Ambrosia artemisiifolia Pollen allergen Amb a 2 precursor epitope
MPRCRFGF

SEQ ID NO: 836 - protein, 15 aa, Artificial Sequence
  Ambrosia artemisiifolia var. elatior Pollen allergen Amb a 3 epitope
CDIKDPIRLEPGGPD

SEQ ID NO: 837 - protein, 31 aa, Artificial Sequence
  Betula pendula pollen allergen Bet v 1 epitope
KAEQVKASKEMGETLLRAVESYLLAHSDAYN

SEQ ID NO: 838 - protein, 20 aa, Artificial Sequence
  Poa pratensis Pollen allergen KBG 60 precursor epitope
AANKYKTFVATFGAASNKAF

SEQ ID NO: 839 - protein, 25 aa, Artificial Sequence
  Lolium perenne Pollen allergen Lol p 2-A (Lol p II-A) epitope
EKGMRNVFDDVVPADFKVGTTYKPE

SEQ ID NO: 840 - protein, 27 aa, Artificial Sequence
  Lolium perenne Pollen allergen Lol p 3 (Lol p III) epitope
KGGMKNVFDEVIPTAFTVGKTYTPEYN

SEQ ID NO: 841 - protein, 12 aa, Artificial Sequence
  Lolium perenne Pollen allergen Lol p VA precursor epitope
AAEGATPEAKYD

SEQ ID NO: 842 - protein, 15 aa, Artificial Sequence
  Phleum pratense Pollen allergen Phl p 1 precursor epitope
APYHFDLSGHAFGAM

SEQ ID NO: 843 - protein, 8 aa, Artificial Sequence
  Zea mays pollen allergen Phl p 11 epitope
RDRARVPL

SEQ ID NO: 844 - protein, 12 aa, Artificial Sequence
  Phleum pratense pollen allergen Phl pI epitope
IPKVPPGPNITA

SEQ ID NO: 845 - protein, 20 aa, Artificial Sequence
  Cryptomeria japonica Polygalacturonase precursor epitope
GQCKWVNGREICNDRDRPTA

SEQ ID NO: 846 - protein, 30 aa, Artificial Sequence
  Parietaria judaica Probable non-specific lipid-transfer protein epitope
QETCGTMVRALMPCLPFVQGKEKEPSKGCC

SEQ ID NO: 847 - protein, 10 aa, Artificial Sequence
  Parietaria judaica Probable non-specific lipid-transfer protein 2 epitope
AEVPKKCDIK

SEQ ID NO: 848 - protein, 30 aa, Artificial Sequence
  Parietaria judaica Probable non-specific lipid-transfer protein 2 precursor epitope
EACGKVVQDIMPCLHFVKGEEKEPSKECCS

SEQ ID NO: 849 - protein, 12 aa, Artificial Sequence
  Solanum lycopersicum Probable pectate lyase P59 epitope
AFNHFGKRLIQR

SEQ ID NO: 850 - protein, 11 aa, Artificial Sequence
  Homo sapiens profilaggrin epitope
GGQGSRHQQAR

SEQ ID NO: 851 - protein, 10 aa, Artificial Sequence
  Cucumis melo profilin epitope
AFRLEEIAAI

SEQ ID NO: 852 - protein, 56 aa, Artificial Sequence
  Glycine max Profilin-1 epitope
WAQSTDFPQFKPEEITAIMNDFNEPGSLAPTGLYLGGTKYMVIQGEPGAVIRGKKG

SEQ ID NO: 853 - protein, 43 aa, Artificial Sequence; identical to SEQ ID NO: 491
  Hevea brasiliensis Pro-hevein precursor epitope

SEQ ID NO: 854 - protein, 15 aa, Artificial Sequence
  Homo sapiens Proliferating cell nuclear antigen epitope
LKYYLAPKIEDEEGS

SEQ ID NO: 855 - protein, 9 aa, Artificial Sequence
  Homo sapiens Proline-rich transmembrane protein 2 epitope
HSEAETGPP

SEQ ID NO: 856 - protein, 15 aa, Artificial Sequence
  Homo sapiens proteasome (prosome, macropain) activator subunit 3 (PA28 gamma; Ki), isoform CRA_a epitope
LDGPTYKRRLDECEE

SEQ ID NO: 857 - protein, 13 aa, Artificial Sequence
  Homo sapiens protein tyrosine phosphatase-like autoantigen epitope
GAHGDTTPEYQDL

SEQ ID NO: 858 - protein, 20 aa, Artificial Sequence
  Homo sapiens protein-arginine deiminase type-4 epitope
AFFPNMVNMLVLGKHLGIPK

SEQ ID NO: 859 - protein, 15 aa, Artificial Sequence
  Homo sapiens proteinase 3 epitope
CATRLFPDFFTRVAL

SEQ ID NO: 860 - protein, 10 aa, Artificial Sequence
  Prunus persica pru p 1 epitope
GKCGVSIPYK

SEQ ID NO: 861 - protein, 15 aa, Artificial Sequence
  Prunus dulcis prunin 1 precursor epitope
EESQQSSQQGRQQEQ

SEQ ID NO: 862 - protein, 15 aa, Artificial Sequence
  Prunus dulcis prunin 2 precursor epitope
DSQPQQFQQQQQQQQ

SEQ ID NO: 863 - protein, 11 aa, Artificial Sequence
  Hesperocyparis arizonica putative allergen Cup a 1 epitope
WRFTRDAFTNG

SEQ ID NO: 864 - protein, 11 aa, Artificial Sequence
  Homo sapiens Putative HTLV-1-related endogenous sequence (p25) epitope
PTRAPSGPRPP

SEQ ID NO: 865 - protein, 8 aa, Artificial Sequence
  Homo sapiens Putative small nuclear ribonucleoprotein polypeptide E-like protein 1 epitope
EIHSKTKS

SEQ ID NO: 866 - protein, 18 aa, Artificial Sequence
  Homo sapiens Receptor tyrosine-protein kinase erbB-2 precursor epitope
PESFDGDPASNTAPLQPE

SEQ ID NO: 867 - protein, 13 aa, Artificial Sequence
  Homo sapiens Receptor-type tyrosine-protein phosphatase-like N precursor epitope
KERLAALGPEGAH

SEQ ID NO: 868 - protein, 11 aa, Artificial Sequence
  Homo sapiens recombinant IgG2 heavy chain epitope
EPQVVTLPPSR

SEQ ID NO: 869 - protein, 20 aa, Artificial Sequence
  Homo sapiens Replication protein A 32 kDa subunit epitope
RSFQNKKSLVAFKIMPLEDM

SEQ ID NO: 870 - protein, 10 aa, Artificial Sequence
  Aspergillus fumigatus Ribonuclease mitogillin precursor epitope
FPTFPDGHDY

SEQ ID NO: 871 - protein, 23 aa, Artificial Sequence
  Homo sapiens ribosomal protein L7 epitope
ELKIKRLRKKFAQKMLRKARRKL

SEQ ID NO: 872 - protein, 14 aa, Artificial Sequence
  Homo sapiens ribosomal protein P2 epitope
SEESDDDMGFGLFD

SEQ ID NO: 873 - protein, 12 aa, Artificial Sequence
  Mangifera indica ripening-related pectate lyase epitope
AYNHFGEGLIQR

SEQ ID NO: 874 - protein, 21 aa, Artificial Sequence
  Homo sapiens RNA binding protein, autoantigenic (hnRNP-associated with lethal yellow homolog (mouse)), isoform CRA_c epitope
GGGGGGSGGGGSGGGGGGGSS

SEQ ID NO: 875 - protein, 21 aa, Artificial Sequence
  Homo sapiens Ro ribonucleoprotein epitope
DGYVWQVTDMNRLHRFLCFGS

SEQ ID NO: 876 - protein, 10 aa, Artificial Sequence
  Hevea brasiliensis Rubber elongation factor protein epitope
AEDEDNQQGQ

SEQ ID NO: 877 - protein, 14 aa, Artificial Sequence
  Homo sapiens S-arrestin epitope
FLGELTSSEVATEV

SEQ ID NO: 878 - protein, 15 aa, Artificial Sequence
  Juglans regia seed storage protein epitope
DDNGLEETICTLRLR

SEQ ID NO: 879 - protein, 15 aa, Artificial Sequence
  Arachis hypogaea seed storage protein SSP2 epitope
CGLRAPQRCDLDVES

SEQ ID NO: 880 - protein, 8 aa, Artificial Sequence
  Gallus gallus serine (or cysteine) proteinase inhibitor, clade B (ovalbumin), member 3 epitope
RPNATYSL

SEQ ID NO: 881 - protein, 8 aa, Artificial Sequence
  Gallus gallus Serum albumin epitope
QSRATLGI

SEQ ID NO: 882 - protein, 17 aa, Artificial Sequence
  Bos taurus Serum albumin precursor epitope
DDSPDLPKLKPDPNTLC

SEQ ID NO: 883 - protein, 8 aa, Artificial Sequence
  Homo sapiens small nuclear ribonucleoprotein epitope
PPPGIRGP

SEQ ID NO: 884 - protein, 8 aa, Artificial Sequence
  Homo sapiens small nuclear ribonucleoprotein B′ epitope
PPPGMRGP

SEQ ID NO: 885 - protein, 24 aa, Artificial Sequence
  Homo sapiens small nuclear ribonucleoprotein D1 polypeptide epitope
KMTLKNREPVQLETLSIRGNRIRY

SEQ ID NO: 886 - protein, 23 aa, Artificial Sequence
  Homo sapiens small nuclear ribonucleoprotein D2 isoform 1 epitope
GKKKSKPVNKDRYISKMFLRGDS

SEQ ID NO: 887 - protein, 8 aa, Artificial Sequence
  Homo sapiens small nuclear ribonucleoprotein F epitope
EEEEDGEM

SEQ ID NO: 888 - protein, 8 aa, Artificial Sequence
  Homo sapiens small nuclear ribonucleoprotein G epitope
WSKAHPPE

SEQ ID NO: 889 - protein, 9 aa, Artificial Sequence
  Homo sapiens small nuclear ribonucleoprotein polypeptide A epitope
AMKISFAKK

SEQ ID NO: 890 - protein, 7 aa, Artificial Sequence
  Homo sapiens small nuclear ribonucleoprotein polypeptide B epitope
PPGMRPP

SEQ ID NO: 891 - protein, 12 aa, Artificial Sequence
  Homo sapiens small nuclear ribonucleoprotein polypeptide B/B′ isoform B epitope
MGRGAPPPGMMG

SEQ ID NO: 892 - protein, 7 aa, Artificial Sequence
  Homo sapiens small nuclear ribonucleoprotein polypeptide C, isoform CRA_b epitope
APGMRPP

SEQ ID NO: 893 - protein, 15 aa, Artificial Sequence
  Homo sapiens small nuclear ribonucleoprotein polypeptide D3 epitope
AARGRGRGMGRGNIF

SEQ ID NO: 894 - protein, 12 aa, Artificial Sequence
  Homo sapiens small nuclear ribonucleoprotein polypeptide N variant epitope
VGRATPPPGIMA

SEQ ID NO: 895 - protein, 23 aa, Artificial Sequence
  Homo sapiens Small nuclear ribonucleoprotein Sm D1 epitope
GRGRGRGRGRGRGRGRGRGGPRR

SEQ ID NO: 896 - protein, 8 aa, Artificial Sequence
  Homo sapiens Small nuclear ribonucleoprotein Sm D2 epitope
EELQKREE

SEQ ID NO: 897 - protein, 8 aa, Artificial Sequence
  Homo sapiens Small nuclear ribonucleoprotein-associated proteins B and B′ epitope
RGVGGPSQ

SEQ ID NO: 898 - protein, 10 aa, Artificial Sequence
  Hevea brasiliensis Small rubber particle protein epitope
AEEVEEERLK

SEQ ID NO: 899 - protein, 20 aa, Artificial Sequence
  Homo sapiens Smoothelin epitope
GSTMMQTKTFSSSSSSKKMG

SEQ ID NO: 900 - protein, 15 aa, Artificial Sequence
  Homo sapiens snRNP polypeptide B epitope
PPGMRPPMGPMGIPP

SEQ ID NO: 901 - protein, 14 aa, Artificial Sequence
  Homo sapiens spectrin, alpha, non-erythrocytic 1 (alpha-fodrin), isoform CRA_e epitope
FQFFQRDAEELEKW

SEQ ID NO: 902 - protein, 43 aa, Artificial Sequence
  Homo sapiens steroid 17-alpha-hydroxylase/17,20 lyase epitope
EVPDDGQLPSLEGIPKVVFLIDSFKVKIKVRQAWREAQAEGST

SEQ ID NO: 903 - protein, 15 aa, Artificial Sequence
  Homo sapiens Sucrase-isomaltase, intestinal epitope
DFTYDQVAFNGLPQF

SEQ ID NO: 904 - protein, 12 aa, Artificial Sequence
  Cryptomeria japonica Sugi basic protein precursor epitope
DALTLRTATNIW

SEQ ID NO: 905 - protein, 48 aa, Artificial Sequence
  Aspergillus fumigatus Superoxide dismutase epitope
YTLPPLPYPYDALQPYISQQIMELHHKKHHQTYVNGLNAALEAQKKAA

SEQ ID NO: 906 - protein, 11 aa, Artificial Sequence
  Homo sapiens T cell receptor beta variable 20 epitope
RSLDFQATTMF

SEQ ID NO: 907 - protein, 18 aa, Artificial Sequence
  Homo sapiens T cell receptor beta variable 5 epitope
ALGQGPQFIFQYYEEEER

SEQ ID NO: 908 - protein, 15 aa, Artificial Sequence
  Homo sapiens Tax1-binding protein 1 epitope
EFKKRFSDATSKAHQ

SEQ ID NO: 909 - protein, 16 aa, Artificial Sequence
  Homo sapiens T-cell receptor beta chain epitope
QPLKEQPALNDSRYCL

SEQ ID NO: 910 - protein, 16 aa, Artificial Sequence
  Homo sapiens T-cell receptor beta chain C region epitope
SATFWQNPRNHFRCQV

SEQ ID NO: 911 - protein, 16 aa, Artificial Sequence
  Homo sapiens T-cell receptor beta chain V region YT35 epitope
CKPISGHNSLFWYRQT

SEQ ID NO: 912 - protein, 10 aa, Artificial Sequence
  Homo sapiens T-cell receptor beta-chain (V1-D-J-C) precursor epitope
SPRSGDLSVY

SEQ ID NO: 913 - protein, 21 aa, Artificial Sequence
  Homo sapiens TCR V-beta 6.1 epitope
LGQGPEFLIYFQGTGAADDSG

SEQ ID NO: 914 - protein, 10 aa, Artificial Sequence
  Homo sapiens TCR V-beta 6.3 epitope
DPISGHVSLF

SEQ ID NO: 915 - protein, 20 aa, Artificial Sequence
  Homo sapiens Thyroglobulin epitope
PPARALKRSLWVEVDLLIGS

SEQ ID NO: 916 - protein, 19 aa, Artificial Sequence
  Homo sapiens Thyroid peroxidase epitope
GLPRLETPADLSTAIASRS

SEQ ID NO: 917 - protein, 15 aa, Artificial Sequence
  Homo sapiens thyroid stimulating hormone receptor epitope
EIIGFGQELKNPQEE

SEQ ID NO: 918 - protein, 16 aa, Artificial Sequence
  Homo sapiens thyroid stimulating hormone receptor variant epitope
EEQEDEIIGFGQELKN

SEQ ID NO: 919 - protein, 20 aa, Artificial Sequence
  Homo sapiens Thyrotropin receptor epitope
GQELKNPQEETLQAFDSHYD

SEQ ID NO: 920 - protein, 15 aa, Artificial Sequence
  Homo sapiens transaldolase 1 epitope
AAAQMPAYQELVEEA

SEQ ID NO: 921 - protein, 20 aa, Artificial Sequence
  Trichophyton rubrum Tri r 2 allergen epitope
DCNGHGTHVAGTVGGTKYGL

SEQ ID NO: 922 - protein, 15 aa, Artificial Sequence
  Homo sapiens trinucleotide repeat containing 6A, isoform CRA_b epitope
AFLSVDHLGGGGESM

SEQ ID NO: 923 - protein, 15 aa, Artificial Sequence
  Homo sapiens trinucleotide repeat containing 6A, isoform CRA_c epitope
WGSSSVGPQALSKSG

SEQ ID NO: 924 - protein, 36 aa, Artificial Sequence
  Homo sapiens tripartite motif-containing 67 epitope
LGGGAGGGGDHADKLSLYSETDSGYGSYTPSLKSPN

SEQ ID NO: 925 - protein, 10 aa, Artificial Sequence
  Triticum aestivum Triticum aestivum proteins epitope
LPQQQIPQQP

SEQ ID NO: 926 - protein, 9 aa, Artificial Sequence
  Penaeus tropomyosin epitope
FLAEEADRK

SEQ ID NO: 927 - protein, 20 aa, Artificial Sequence
  Homo sapiens TSHR protein epitope
CHQEEDFRVTCKDIQRIPSL

SEQ ID NO: 928 - protein, 14 aa, Artificial Sequence
  Homo sapiens tubulin beta-6 chain epitope
AACDPRHGRYLTVA

SEQ ID NO: 929 - protein, 10 aa, Artificial Sequence
  Homo sapiens tumor necrosis factor ligand superfamily member 6 epitope
EWEDTYGIVL

SEQ ID NO: 930 - protein, 20 aa, Artificial Sequence
  Paralichthys olivaceus type 1 collagen alpha 2 epitope
MKGLRGHPGLQGMPGPSGPS

SEQ ID NO: 931 - protein, 10 aa, Artificial Sequence
  Triticum aestivum type 1 non-specific lipid transfer protein precursor epitope
ARGTPLKCGV

SEQ ID NO: 932 - protein, 5 aa, Artificial Sequence
  Homo sapiens U1 small nuclear ribonucleoprotein 70 kDa epitope
ERKRR

SEQ ID NO: 933 - protein, 26 aa, Artificial Sequence
  Homo sapiens U1 small nuclear ribonucleoprotein A epitope
AGAARDALQGFKITQNNAMKISFAKK

SEQ ID NO: 934 - protein, 8 aa, Artificial Sequence
  Homo sapiens U1 small nuclear ribonucleoprotein C epitope
PAPGMRPP

SEQ ID NO: 935 - protein, 18 aa, Artificial Sequence
  Anisakis simplex UA3-recognized allergen epitope
MCQCVQKYGTEFCKKRLA

SEQ ID NO: 936 - protein, 9 aa, Artificial Sequence
  Homo sapiens unnamed protein product epitope
AFQQGKIPP

SEQ ID NO: 937 - protein, 8 aa, Artificial Sequence
  Juglans nigra vicilin seed storage protein epitope
SFEDQGRR

SEQ ID NO: 938 - protein, 15 aa, Artificial Sequence
  Anacardium occidentale Vicilin-like protein epitope
AIMGPPTKFSFSLFL

SEQ ID NO: 939 - protein, 10 aa, Artificial Sequence
  Juglans regia vicilin-like protein precursor epitope
DQRSQEERER

SEQ ID NO: 940 - protein, 20 aa, Artificial Sequence
  Homo sapiens Vimentin epitope
RLRSSVPGVRLLQDSVDFSL

SEQ ID NO: 941 - protein, 15 aa, Artificial Sequence
  Homo sapiens von Willebrand factor epitope
HCQICHCDVVNLTCE

SEQ ID NO: 942 - protein, 45 aa, Artificial Sequence
  Homo sapiens von Willebrand factor-cleaving protease precursor epitope
PSHFQQSCLQALEPQAVSSYLSPGAPLKGRPPSPGFQRQRQRQRR

SEQ ID NO: 943 - protein, 15 aa, Artificial Sequence
  Homo sapiens XRCC4 protein epitope
VSKDDSIISSLDVTD

SEQ ID NO: 944 - protein, 8 aa, Artificial Sequence
  OVA epitope
SIINFEKL

(19) United States
(12) Patent Application Publication
(10) Pub. No.: US 2013/0058902 A1
(43) Pub. Date: Mar. 7, 2013

(54) DENDRITIC CELL SUBSETS FOR GENERATING INDUCED TOLEROGENIC DENDRITIC CELLS AND RELATED COMPOSITIONS AND METHODS

(71) Applicants: Takashi Kei Kishimoto, Lexington, MA (US); Roberto A. Maldonado, Jamaica Plain, MA (US)

(72) Inventors: Takashi Kei Kishimoto, Lexington, MA (US); Roberto A. Maldonado, Jamaica Plain, MA (US)

(73) Assignee: Selecta Biosciences, Inc., Watertown, MA (US)

(21) Appl. No.: 13/457,900

(22) Filed: Apr. 27, 2012

Related U.S. Application Data
(60) Provisional application No. 61/531,103, filed on Sep. 6, 2011.
(60) Provisional application No. 61/531,106, filed on Sep. 6, 2011.
(60) Provisional application No. 61/531,109, filed on Sep. 6, 2011.
(60) Provisional application No. 61/531,112, filed on Sep. 6, 2011.
(60) Provisional application No. 61/531,115, filed on Sep. 6, 2011.
(60) Provisional application No. 61/531,121, filed on Sep. 6, 2011.
(60) Provisional application No. 61/531,124, filed on Sep. 6, 2011.
(60) Provisional application No. 61/531,127, filed on Sep. 6, 2011.
(60) Provisional application No. 61/531,131, filed on Sep. 6, 2011.
(60) Provisional application No. 61/531,140, filed on Sep. 6, 2011.
(60) Provisional application No. 61/531,231, filed on Sep. 6, 2011.

(51) Int. Cl. C12N 5/0784; A61P 37/06; A61K 35/14

(52) U.S. Cl. 424/93.7; 435/325; 435/375

(57) ABSTRACT

Disclosed are induced tolerogenic dendritic cells (itDCs) produced from dendritic cell subsets that possess a desired physiological characteristic, as well as related compositions and methods.

### RELATED APPLICATIONS

This application claims the benefit under 35 U.S.C. §119 of U.S. provisional application 61/531,103; U.S. provisional application 61/531,106; U.S. provisional application 61/531,109; U.S. provisional application 61/531,112; U.S. provisional application 61/531,115; U.S. provisional application 61/531,121; U.S. provisional application 61/531,124; U.S. provisional application 61/531,127; U.S. provisional application 61/531,131; U.S. provisional application 61/531,140; and U.S. provisional application 61/531,231; all filed Sep. 6, 2011, the entire contents of each of which are incorporated herein by reference.

### FIELD OF THE INVENTION

This invention relates to methods of generating induced tolerogenic dendritic cell (itDC) compositions from dendritic cell subsets that possess a desired physiological characteristic. The itDCs produced or the cells from these subsets can be combined with antigen to produce antigen-specific itDCs. The antigens may comprise MHC Class I-restricted and/or MHC Class II-restricted and/or B cell epitopes. The itDCs can be used to shift to tolerogenic immune responses specific to antigens. The methods and compositions provided, therefore, can be used to generate a tolerogenic immune response in a subject that is experiencing or at risk of experiencing undesired immune responses against an antigen.

### BACKGROUND OF THE INVENTION

Conventional strategies for generating immunosuppression associated with an undesired immune response are based on broad-acting immunosuppressive drugs. Additionally, in order to maintain immunosuppression, immunosuppressant drug therapy is generally a life-long proposition. Unfortunately, the use of broad-acting immunosuppressants are associated with a risk of severe side effects, such as tumors, infections, nephrotoxicity and metabolic disorders. Accordingly, new immunosuppressant therapies would be beneficial.

### SUMMARY OF THE INVENTION

In one aspect, a method comprising isolating cells of a dendritic cell subset that possess a desired physiological characteristic, and generating induced tolerogenic dendritic cells (itDCs) from the cells of the dendritic cell subset is provided.

In one embodiment, the cells of the dendritic cell subset are XCR1+ dendritic cells. In another embodiment, the cells of the dendritic cell subset are plasmacytoid dendritic cells. In another embodiment, the cells of the dendritic cell subset are CD103+ dendritic cells. In another embodiment, the cells of the dendritic cell subset are not enriched for XCR1+ dendritic cells and/or plasmacytoid and/or CD103+ dendritic cells.

In another embodiment, the cells of the dendritic cell subset are isolated from blood, cord blood, lymphatic fluid, lymph nodes, bone marrow or spleen. In another embodiment, the cells of the dendritic cell subset are cells differentiated in vitro.

In another embodiment, the generating comprises contacting the cells of the dendritic cell subset ex vivo with at least one agent that promotes respirostatic tolerance. In another embodiment, the generating comprises contacting the cells of the dendritic cell subset ex vivo for less than 10 h with a purinergic receptor antagonist, an mTOR inhibitor, a statin or an agent that disrupts mitochondrial electron transport. In another embodiment, the generating comprises contacting the cells of the dendritic cell subset ex vivo with at least one agent that causes the cells to increase expression of costimulatory molecules while retaining a tolerogenic phenotype upon stimulation with at least one TLR agonist. In another embodiment, the generating comprises contacting the cells of the dendritic cell subset ex vivo with at least one agent that causes the cells to have the characteristic of i) inducing Foxp3 expression in naïve T cells ex vivo, ii) deleting effector T cells ex vivo and/or iii) converting FoxP3− effector T cells to FoxP3+ effector T cells ex vivo. In another embodiment, the generating comprises contacting dendritic cells differentiated in vitro with at least one agent that causes the cells to have the characteristic of i) inducing Foxp3 expression in naïve T cells ex vivo, ii) deleting effector T cells ex vivo and/or iii) converting FoxP3− effector T cells to FoxP3+ effector T cells ex vivo. In another embodiment, the cells are treated for 1, 2, 3, 4, 5, 6, 7 or more days. In another embodiment, the generating comprises combining the differentiated dendritic cells or cells of the dendritic cell subset with an antigen. This may occur, before or after the treatment with the at least one agent.

In another embodiment, the antigen comprises one or more epitopes of a therapeutic protein, a transplantable graft, an autoantigen or an allergen, or is associated with an inflammatory disease, an autoimmune disease, an allergy, organ or tissue rejection or graft versus host disease. In another embodiment, the one or more epitopes comprise MHC Class I-restricted and/or MHC Class II-restricted and/or B cell epitopes. In another embodiment, the antigen comprises a therapeutic protein, an autoantigen or an allergen, or is associated with a transplantable graft, an inflammatory disease, an autoimmune disease, an allergy, organ or tissue rejection or graft versus host disease.

In another embodiment, the method further comprises collecting the itDCs. In another embodiment, the method further comprises making a dosage form comprising the collected itDCs. In another embodiment, the method further comprises making the collected itDCs available or dosage form available to a subject for administration. In another embodiment, the method further comprises including a transplantable graft or therapeutic protein with the collected itDCs or dosage form. In another embodiment, the method further comprises administering the itDCs or dosage form to a subject. In another embodiment, the methods further comprises assessing the generation of an undesired immune response or a desired immune response with the itDCs. In one embodiment, the assessing is performed in vitro. In another embodiment, the assessing is performed in vivo. In another embodiment, the assessing is performed with a sample obtained from the subject.

In another embodiment, the itDCs are in or are administered in an amount effective to reduce the generation of an undesired immune response or generate a desired immune response.

In another embodiment, the undesired immune response is the undesired proliferation and/or activity of antigen-specific CD4+ and/or CD8+ T cells. In another embodiment, the undesired immune response is the undesired production of antigen-specific antibodies. In another embodiment, the desired immune response is the generation of antigen-specific regulatory T cells or regulatory B cells. In another embodiment, the regulatory T cells are CD4+ and/or CD8+ regulatory T cells.

In another aspect, a method comprising administering to a subject dendritic cell subset-enriched itDCs in an amount effective to reduce the generation of an undesired immune response or to generate a desired immune response in the subject is provided. In another aspect, a method comprising reducing the generation of an undesired immune response or generating a desired immune response in a subject by administering dendritic cell subset-enriched itDCs to the subject is provided. In another aspect, a method comprising administering to a subject dendritic cell subset-enriched itDCs according to a protocol that was previously shown to reduce the generation of an undesired immune response or to generate a desired immune response in one or more test subjects is provided.

In another embodiment, the method further comprises providing or identifying the subject. In another embodiment, the method further comprises assessing the immune response in the subject prior to and/or after the administration of the dendritic cell subset-enriched itDCs.

In another embodiment, the undesired immune response is the undesired proliferation and/or activity of antigen-specific CD4+ and/or CD8+ T cells. In another embodiment, the undesired immune response is the undesired production of antigen-specific antibodies. In another embodiment, the desired immune response is the generation of antigen-specific regulatory T cells or regulatory B cells. In another embodiment, the regulatory T cells are CD4+ and/or CD8+ regulatory T cells.

In another embodiment, the dendritic cell subset-enriched itDCs are enriched for XCR1+ dendritic cells. In another embodiment, the dendritic cell subset-enriched itDCs are enriched for plasmacytoid dendritic cells. In another embodiment, the dendritic cell subset-enriched itDCs are enriched for CD103+ dendritic cells. In another embodiment, the dendritic cell subset-enriched itDCs are not enriched for XCR1+ dendritic cells and/or plasmacytoid and/or CD103+ dendritic cells. In another embodiment, the dendritic cell subset-enriched itDCs are enriched for BDCA1+ DCs, XCR1(BDCA3)+DCs, plasmacytoid DCs, BDCA1+ DCs negative for CD14, CD16, and CD19 and positive for HLA-DR and BDCA1, XCR1+ DCs positive for HLA-DR and XCR1(BDCA3), and/or plasmacytoid DCs positive for HLA-DR and CD123.

In another embodiment, the dendritic cell subset-enriched itDCs are specific for an antigen. In another embodiment, the antigen comprises a therapeutic protein, a transplantable graft, an autoantigen or an allergen, or is associated with an inflammatory disease, an autoimmune disease, an allergy, organ or tissue rejection or graft versus host disease.

In another embodiment, the subject has or is at risk of having an inflammatory disease, an autoimmune disease, an allergy, organ or tissue rejection or graft versus host disease. In another embodiment, the subject has undergone or will undergo transplantation. In another embodiment, the subject has or is at risk of having an undesired immune response against a therapeutic protein that is being administered or will be administered to the subject.

In another embodiment, the method further comprises administering a transplantable graft or therapeutic protein, In another embodiment, one or more maintenance doses of the itDCs are administered to the subject.

In another embodiment, the administering of the antigen-specific itDCs or the transplantable graft or therapeutic protein, when administered as a cell-based therapy, is by parenteral, intraarterial, intranasal or intravenous administration or by injection to lymph nodes or anterior chamber of the eye or by local administration to an organ or tissue of interest. In another embodiment, the administering is by subcutaneous, intrathecal, intraventricular, intramuscular, intraperitoneal, intracoronary, intrapancreatic, intrahepatic or bronchial injection.

In another aspect, a method comprising administering the dendritic cell subset-enriched itDCs produced by any of the methods provided herein is provided.

In another aspect, a composition comprising dendritic cell subset-enriched itDCs is provided. In another embodiment, the dendritic cell subset-enriched itDCs are enriched for XCR1+ dendritic cells. In another embodiment, the dendritic cell subset-enriched itDCs are enriched for plasmacytoid dendritic cells. In another embodiment, the dendritic cell subset-enriched itDCs are enriched for CD103+ dendritic cells. In another embodiment, the dendritic cell subset-enriched itDCs are not enriched for XCR1+ dendritic cells and/or plasmacytoid and/or CD103+ dendritic cells. In another embodiment, the dendritic cell subset-enriched itDCs are enriched for BDCA1+ DCs, XCR1(BDCA3)+DCs, plasmacytoid DCs, BDCA1+ DCs negative for CD14, CD16, and CD19 and positive for HLA-DR and BDCA1, XCR1+ DCs positive for HLA-DR and XCR1(BDCA3), and/or plasmacytoid DCs positive for HLA-DR and CD123.

In another embodiment, the dendritic cell subset-enriched itDCs are also specific for an antigen. In another embodiment, the antigen comprises a therapeutic protein, a transplantable graft, an autoantigen or an allergen, or is associated with an inflammatory disease, an autoimmune disease, an allergy, organ or tissue rejection or graft versus host disease.

In another aspect, a composition comprising the dendritic cell subset-enriched itDCs produced by any of the methods provided herein is provided. In another aspect, a composition comprising dendritic cell subset-enriched itDCs, wherein the dendritic cell subset-enriched itDCs are as defined in any of the compositions and methods provided herein is provided.

In another embodiment, the composition further comprises a transplantable graft or therapeutic protein. In another embodiment, the composition further comprises a pharmaceutically acceptable excipient.

In another aspect, a dosage form comprising any of the compositions provided herein is provided.

In another aspect, a process for producing a composition comprising dendritic cell subset-enriched itDCs comprising the steps of isolating cells of a dendritic cell subset that possess a desired physiological characteristic, and generating induced tolerogenic dendritic cells (itDCs) from the cells of the dendritic cell subset is provided. In another embodiment, the process comprises the steps as defined in any of the methods provided herein.

In another aspect, subset-enriched itDCs or a dosage form comprising subset-enriched itDCs obtainable by any of the methods or processes provided herein are provided.

In another aspect, any of the subset-enriched itDCs, compositions or dosage forms provided herein may be for use in therapy or prophylaxis.

In another aspect, any of the subset-enriched itDCs, compositions or dosage forms provided herein may be for use in a method of reducing the generation of an undesired immune response or generating a desired immune response in a subject, for the treatment or prophylaxis of an inflammatory disease, an autoimmune disease, an allergy, organ or tissue rejection or graft versus host disease or in any of the methods provided herein.

In another aspect, a use of any of the subset-enriched itDCs, compositions or dosage forms provided herein for the manufacture of a medicament for use in a method of reducing the generation of an undesired immune response or generating a desired immune response in a subject, for the treatment or prophylaxis of an inflammatory disease, an autoimmune disease, an allergy, organ or tissue rejection or graft versus host disease or in any of the methods provided herein is provided.

In another aspect, a dosage form comprising any of the compositions provided herein is provided.

In embodiments of any of the compositions provided herein, the composition may further comprise an agent that enhances the migratory behavior (e.g., to an organ or tissue of interest) of the itDCs, including the antigen-specific itDCs. In embodiments of any of the methods provided herein, the method may further comprise administering an agent that enhances the migratory behavior of the itDCs.

In embodiments of any of the compositions and methods provided herein, the itDCs are circulating itDCs. In other embodiments, the circulating itDCs are CD103+, CD11b+, XCR1+ or plasmacytoid itDCs and/or are not CD8α+. In other embodiments, the circulating itDCs are CD103+ itDCs. In embodiments of any of the compositions and methods provided herein, the itDCs are not XCR1+ and/or CD8α+ itDCs. In other embodiments of any of the composition and methods provided herein, the itDCs are not derived from XCR1+ and/or CD8α+ DCs.

In an embodiment of any of the compositions and methods provided herein, the antigen-specific itDCs or antigens comprise substantially no B cell epitopes.

In an embodiment of any of the compositions and methods provided herein, the antigens are peptides. Such antigens, in some embodiments, comprise at least an epitope as described anywhere herein but may also comprise additional amino acids that flank one or both ends of the epitope. In embodiments, the antigens comprise a whole antigenic protein. These antigens may be combined with the itDCs, or precursors thereof, to ultimately form the antigen-specific itDCs.

In an embodiment of any of the compositions and methods provided herein, the antigen comprise multiple types of antigens. In some embodiments, the antigens comprise multiple types of peptides that comprise the same epitopic sequence or different epitopic sequences.

### BRIEF DESCRIPTION OF THE FIGURES

FIG. 1 demonstrates that antigen-specific itDCs of a specific subset effectively reduce the specific killing of cells expressing antigen.

FIG. 2 demonstrates that antigen-specific itDCs of a specific subset effectively reduce the production of antigen-specific antibodies.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a cell” includes a mixture of two or more such cells or a plurality of such cells, reference to “a DNA molecule” includes a mixture of two or more such DNA molecules or a plurality of such DNA molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

### A. INTRODUCTION

It has been found that subsets of dendritic cells that possess a desired physiological characteristic can be used to generate itDCs. It has also been found that itDCs generated from a dendritic cell subsets can also be loaded with antigen and used to modulate immune responses. Thus, specific subsets can be chosen depending on the context for immune modulation depending on the desired characteristics. As shown in the Examples, CD103+ itDCs loaded with antigen from ovalbumin were successfully used to reduce the antigen-specific killing of cells as well as the production of antigen-specific antibodies. Such a subset has a broad distribution and would be expected to be useful in a number of treatment contexts. It is expected that other subsets can also be used to generate itDCs, such as antigen-specific itDCs, and modulate immune responses in some embodiments.

Generally, the itDCs generated are dendritic cell subset-enriched itDCs. The itDCs provided herein can result in the stimulation of beneficial immune responses, such as beneficial tolerogenic immune response development specific to antigens. Antigen-specific itDCs may be produced by combining cells of the dendritic cell subset or the itDCs produced therefrom with antigen. The itDCs, including the antigen-specific itDCs, provided are expected to be useful, for example, for promoting tolerogenic immune responses in subjects who have or are at risk of having an allergy, autoimmune disease, an inflammatory disease, organ or tissue rejection or graft versus host disease. The itDCs are also expected to be useful for promoting tolerogenic immune responses in subjects who have undergone or will undergo transplantation. This invention is also expected to be useful for promoting tolerogenic immune responses in subjects that have received, are receiving or will receive a therapeutic protein against which undesired immune responses are generated or are expected to be generated. The present invention, in some embodiments, prevents or suppresses such undesired immune responses that may neutralize the beneficial effect of certain therapeutic treatments.

The inventors have unexpectedly and surprisingly discovered that the problems and limitations noted above can be overcome by practicing the invention disclosed herein. In particular, the inventors have unexpectedly discovered that it is possible to produce itDCs by isolating cells of a dendritic cell subset that possesses a desired physiological characteristic and generating itDCs from the cells of the dendritic cell subset. In embodiments, the dendritic cell subset or itDCs produced therefrom can be combined with antigen. The antigens may comprise MHC Class I-restricted and/or MHC Class II-restricted and/or B cell epitopes. In some embodiments, the antigen of the antigen-specific itDCs comprise substantially no B cell epitopes, such as when the presence of such B cell epitopes may result in or exacerbate an undesired immune response.

It follows that antigen-specific itDCs can also be generated and that these antigen-specific itDCs can generate tolerogenic immune responses specific to antigens. The antigens may be combined in the form of the antigen itself or a fragment or derivative thereof or in the form of one or more cells that express the antigen. The antigen, therefore, may be in the form of live cells in their native cellular form or they may be processed into a form suitable for uptake before combining. In embodiments, the processing comprises obtaining a cell suspension, a cell lysate, a cell homogenate, cell exosomes, cell debris, conditioned medium, or a partially purified protein preparation from the cells that express the antigen. In other embodiments, the processing comprises obtaining proteins, protein fragments, fusion proteins, peptides, peptide mimeotypes, altered peptides, fusion peptides from materials obtained from the cells. In other embodiments, the antigen is combined in the presence of an agent that enhances the uptake, processing or presentation of antigens. In some embodiments, naïve cells of a dendritic cell subset, or itDCs produced therefrom (and in embodiments also naïve), are combined with the antigens as provided above and elsewhere herein.

The itDCs, including the antigen-specific itDCs, can be administered to a subject in order to ameliorate an undesired immune response or to generate a desired immune response. In one aspect, a method comprising administering to a subject dendritic cell subset-enriched itDCs in an amount effective to reduce the generation of an undesired immune response or to generate a desired immune response in the subject is provided. In another aspect, a method comprising reducing the generation of an undesired immune response or generating a desired immune response in a subject by administering dendritic cell subset-enriched itDCs to the subject is provided. In yet another aspect, a method comprising administering to a subject dendritic cell subset-enriched itDCs according to a protocol that was previously shown to reduce the generation of an undesired immune response or to generate a desired immune response in one or more test subjects is provided. The methods provided, in some embodiments, may further comprise administering a transplantable graft or therapeutic protein.

Compositions and dosage form of the itDCs are also provided. In embodiments, the compositions may also include a therapeutic protein or a transplantable graft. In other embodiments, the therapeutic protein or transplantable graft may be administered to a subject prior to, concomitantly with or after the administration of the itDCs. In embodiments, the itDCs provided may be administered as one or more maintenance doses, such as to a subject that has been receiving, is receiving or will receive a therapeutic protein or transplantable graft or that is exposed to or will be exposed to an allergen. In embodiments, the compositions provided are administered such that the generation of tolerogenic immune response occurs for a certain length of time. Examples of such lengths of time are provided elsewhere herein.

The compositions and dosage forms provided can be administered to any subject in need of undesired immune response reduction or desired immune response generation. Such a subject may be one that has or is at risk of having an inflammatory disease, an autoimmune disease, an allergy, organ or tissue rejection or graft versus host disease. Such a subject may also be one that has undergone or will undergo transplantation. Such a subject may also be one that has experienced, is experiencing or is expected to experience an undesired immune response to a therapeutic protein.

The invention will now be described in more detail below.

### B. DEFINITIONS

“Administering” or “administration” means providing a material to a subject in a manner that is pharmacologically useful.

“Allergens” are any substances that can cause an undesired (e.g., a Type 1 hypersensitive) immune response (i.e., allergic response or reaction) in a subject. Allergens include, but are not limited to, plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Food allergens include, but are not limited to milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Insect sting allergens include allergens that are or are associated with bee stings, wasp stings, hornet stings, yellow jacket stings, etc. Insect allergens also include house dust mite allergens (e.g., Der P1 antigen) and cockroach allergens. Drug allergens include allergens that are or are associated with antibiotics, NSAIDs, anaesthetics, etc. Pollen allergens include grass allergens, tree allergens, weed allergens, flower allergens, etc. Subjects that develop or are at risk of developing an undesired immune response to any of the allergens provided herein may be treated with any of the compositions and methods provided herein. Subjects that may be treated with any of the compositions and methods provided also include those who have or are at risk of having an allergy to any of the allergens provided. “Allergens associated with an allergy” are allergens that generate an undesired immune response that results in, or would be expected by a clinician to result in, alone or in combination with other allergens, an allergic response or reaction or a symptom of an allergic response or reaction in a subject.

It is intended that epitopes of an allergen may be presented by the itDCs as provided herein. The epitopes themselves may be combined with the DCs or proteins, polypeptides, peptides, etc. that comprise these epitopes may be combined with the DCs. Thus an allergen itself or a portion thereof that comprises the epitopes may be combined with the DCs in the methods and compositions provided herein. The epitopes in the compositions and methods provided herein can be presented for recognition by cells of the immune system such as by, for example, T cells. Such epitopes may normally be recognized by and trigger an immune response in a T cell via presentation by a major histocompatability complex molecule (MHC), but in the compositions provided herein the presentation of such epitopes by the itDCs can result in tolerogenic immune responses. In some embodiments, substantially no B cell epitopes are presented, such as when the inclusion of the B cell epitopes would exacerbate an undesired immune response and thus, the allergens or portions thereof, in some embodiments, substantially comprise no B cell epitopes.

An “allergy” also referred to herein as an “allergic condition,” is any condition where there is an undesired (e.g., a Type 1 hypersensitive) immune response (i.e., allergic response or reaction) to a substance. Such substances are referred to herein as allergens. Allergies or allergic conditions include, but are not limited to, allergic asthma, hay fever, hives, eczema, plant allergies, bee sting allergies, pet allergies, latex allergies, mold allergies, cosmetic allergies, food allergies, allergic rhinitis or coryza, topic allergic reactions, anaphylaxis, atopic dermatitis, hypersensitivity reactions and other allergic conditions. The allergic reaction may be the result of an immune reaction to any allergen. In some embodiments, the allergy is a food allergy. Food allergies include, but are not limited to, milk allergies, egg allergies, nut allergies, fish allergies, shellfish allergies, soy allergies or wheat allergies.

“Amount effective” in the context of a composition or dosage form for administration to a subject refers to an amount of the composition or dosage form that produces one or more desired immune responses in the subject, for example, a reduction in the generation of an undesired immune response, such as undesired CD8+ T cell immune responses. Therefore, in some embodiments, an amount effective is any amount of a composition provided herein that produces one or more of these desired immune responses. This amount can be for in vitro or in vivo purposes. For in vivo purposes, the amount can be one that a clinician would believe may have a clinical benefit for a subject in need of antigen-specific tolerization. Such subjects include those that have or are at risk of having an inflammatory disease, an autoimmune disease, an allergy, organ or tissue rejection or graft versus host disease. Such subjects also include those that have undergone or will undergo transplantation. Such subjects further include those that have experienced, are experiencing or are expected to experience an undesired immune response against a therapeutic protein.

Amounts effective can involve only reducing the level of an undesired immune response, although in some embodiments, it involves preventing an undesired immune response altogether. Amounts effective can also involve delaying the occurrence of an undesired immune response. An amount that is effective can also be an amount of a composition provided herein that produces a desired therapeutic endpoint or a desired therapeutic result. Amounts effective, preferably, result in a tolerogenic immune response in a subject to an antigen. The achievement of any of the foregoing can be monitored by routine methods.

In some embodiments of any of the compositions and methods provided, the amount effective is one in which the desired immune response persists in the subject for at least 1 week, at least 2 weeks, at least 1 month, at least 2 months, at least 3 months, at least 4 months, at least 5 months, at least 6 months, at least 9 months, at least 1 year, at least 2 years, at least 5 years, or longer. In other embodiments of any of the compositions and methods provided, the amount effective is one which produces a measurable desired immune response, for example, a measurable decrease in an immune response (e.g., to a specific antigen), for at least 1 week, at least 2 weeks, at least 1 month, at least 2 months, at least 3 months, at least 4 months, at least 5 months, at least 6 months, at least 9 months, at least 1 year, at least 2 years, at least 5 years, or longer.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

In some embodiments, doses of the itDCs in the compositions of the invention can range from a single cell to about 10¹² cells. In some embodiments, the number of itDCs administered to a subject can range from about 1 cell/kg body weight to about 10⁸ cells/kg. In some embodiments, the number of itDCs administered is the smallest number that produces a desired immune response in the subject. In some embodiments, the dose is the largest number of itDCs that can be administered without generating an undesired effect in the subject, for example, an undesired side effect. Useful doses include, in some embodiments, cell populations of greater than 10², 10³, 10⁴, 10⁵, 10⁶, 10⁷, 10⁸, 10⁹ or 10¹⁰ itDCs per dose. Other examples of useful doses include from about 1×10⁴ to about 1×10⁶, about 1×10⁶ to about 1×10⁸ or about 1×10⁸ to about 1×10¹⁰ itDCs per dose.

“Antigen” means a B cell antigen or T cell antigen. “Type(s) of antigens” means molecules that share the same, or substantially the same, antigenic characteristics. In some embodiments, antigens may be proteins, polypeptides, peptides, lipoproteins, glycolipids, polynucleotides, polysaccharides or are contained or expressed in, on or by cells. In some embodiments, such as when the antigens are not well defined or characterized, the antigens may be contained within a cell or tissue preparation, cell debris, cell exosomes, conditioned media, etc. and are provided as such. An antigen can be combined with the DCs in the same form as what a subject is exposed to that causes an undesired immune response but may also be a fragment or derivative thereof. When a fragment or derivative, however, a desired immune response to the form encountered by such a subject is the preferable result with the compositions and methods provided.

“Antigen-specific” refers to any immune response that results from the presence of the antigen, or portion thereof, or that generates molecules that specifically recognize or bind the antigen, such as antibodies that specifically bind the antigen or portion thereof. For example, where the immune response is antigen-specific T cell proliferation and/or activity, the proliferation and/or activity results from recognition of the antigen, or portion thereof, generally in complex with MHC molecules.

“Antigens associated” with a disease, disorder or condition provided herein are antigens that can generate an undesired immune response against, as a result of, or in conjunction with, the disease, disorder or condition; the cause of the disease, disorder or condition (or a symptom or effect thereof); and/or can generate an undesired immune response that is a symptom, result or effect of the disease, disorder or condition. Preferably, in some embodiment use of an antigen associated with a disease, disorder or condition, etc. on the itDCs in the compositions and methods provided herein will lead to a tolerogenic immune response against the antigen and/or the cells in, by or on which the antigen is expressed. In one embodiment, the antigen associated with a disease, disorder or condition, etc. described herein can when presented by the described itDCs lead to a tolerogenic immune response that is specific to the disease, disorder or condition, etc. The antigens can be in the same form as expressed in a subject with the disease, disorder or condition but may also be a fragment or derivative thereof. When a fragment or derivative, however, a desired immune response to the form expressed in such a subject is the preferable result with the compositions and methods provided.

In one embodiment, the antigen is an antigen associated with an inflammatory disease, autoimmune disease, organ or tissue rejection or graft versus host disease. Such antigens include autoantigens, such as myelin basic protein, collagen (e.g., collagen type 11), human cartilage gp 39, chromogranin A, gp130-RAPS, proteolipid protein, fibrillarin, nuclear proteins, nucleolar proteins (e.g., small nucleolar protein), thyroid stimulating factor receptor, histones, glycoprotein gp 70, ribosomal proteins, pyruvate dehydrogenase dehydrolipoamide acetyltransferase, hair follicle antigens, human tropomyosin isoform 5, mitochondrial proteins, pancreatic β-cell proteins, myelin oligodendrocyte glycoprotein, insulin, glutamic acid decarboxylase (GAD), gluten and fragments or derivatives thereof. Other autoantigens are provided in Table 1 below.

Antigens also include those associated with organ or tissue rejection. Examples of such antigens include, but are not limited to, antigens from allogeneic cells, e.g., antigens from an allogeneic cell extract, and antigens from other cells, such as endothelial cell antigens.

Antigens also include those associated with an allergy. Such antigens may include allergens, which are described elsewhere herein.

Antigens also include those associated with a transplantable graft. Such antigens are associated with a transplantable graft, or an undesired immune response in a recipient of a transplantable graft that is generated as a result of the introduction of the transplantable graft in the recipient, that can be presented for recognition by cells of the immune system and that can generate an undesired immune response. Transplant antigens include those associated with organ or tissue rejection or graft versus host disease. Transplant antigens may be obtained or derived from cells of a biological material or from information related to a transplantable graft. Transplant antigens generally include proteins, polypeptides, peptides, lipoproteins, glycolipids, polynucleotides or are contained or expressed in cells. Information related to a transplantable graft is any information about a transplantable graft that can be used to obtain or derive transplant antigens. Such information includes information about antigens that would be expected to be present in or on cells of a transplantable graft such as, for example, sequence information, types or classes of antigens and/or their MHC Class I, MHC Class II or B cell presentation restrictions. Such information may also include information about the type of transplantable graft (e.g, autograft, allograft, xenograft), the molecular and cellular composition of the graft, the bodily location from which the graft is derived or to which the graft to be transplanted (e.g., whole or partial organ, skin, bone, nerves, tendon, neurons, blood vessels, fat, cornea, etc.).

Antigens also include antigens associated with a therapeutic protein that can be presented for recognition by cells of the immune system and that can generate an undesired immune response against the therapeutic protein. Therapeutic protein antigens generally include proteins, polypeptides, peptides, lipoproteins, or are contained or expressed in, by or on cells.

Antigens can be antigens that are fully defined or characterized. However, in some embodiments, an antigen is not fully defined or characterized. Antigens, therefore, also include those that are contained within a cell or tissue preparation, cell debris, cell exosome or conditioned media and can be delivered in such form in some embodiments.

“Antigen-specific itDCs” refers to itDCs that present antigens and modulate immune responses specific to the antigens. As provided herein, the antigen-specific itDCs are those obtained or derived from the cells of a dendritic cell subset and that present antigen after the cells of a dendritic cell subset or itDCs produced therefrom are combined with antigen. Such antigens may comprise MHC Class I-restricted and/or MHC Class II-restricted and/or B cell epitopes. In some embodiments, antigen-specific itDCs are generated by antigen-loading of itDCs, for example, naïve itDCs that have not been exposed to an antigen. In some embodiments, antigen-specific itDCs are administered to a subject and induce a tolerogenic reaction to the antigen in the subject. Antigen-loading is achieved, in some embodiments, by combining itDCs with the antigen (provided in any of the forms provided herein). In some embodiments, the antigens combined with the itDCs are externally loadable and do not require uptake and intracellular processing by the itDCs for presentation.

“Assessing an immune response” refers to any measurement or determination of the level, presence or absence, reduction, increase in, etc. of an immune response in vitro or in vivo. Such measurements or determinations may be performed on one or more samples obtained from a subject. Such assessing can be performed with any of the methods provided herein or otherwise known in the art.

An “at risk” subject is one in which a health practitioner believes has a chance of having a disease, disorder or condition as provided herein, or is one a health practitioner believes has a chance of experiencing an undesired immune response as provided herein.

An “autoimmune disease” is any disease where the immune system mounts an undesired immune response against self (e.g., one or more autoantigens). In some embodiments, an autoimmune disease comprises an aberrant destruction of cells of the body as part of the self-targeted immune response. In some embodiments, the destruction of self manifests in the malfunction of an organ, for example, the colon or pancreas. Examples of autoimmune diseases are described elsewhere herein. Additional autoimmune diseases will be known to those of skill in the art and the invention is not limited in this respect.

“B cell antigen” means any antigen that is or recognized by and triggers an immune response in a B cell (e.g., an antigen that is specifically recognized by a B cell or a receptor thereon). In some embodiments, an antigen that is a T cell antigen is also a B cell antigen. In other embodiments, the T cell antigen is not also a B cell antigen. B cell antigens include, but are not limited to proteins, peptides, etc.

“Cells processed into a form suitable for uptake by the itDCs” refers to cells that were treated or processed to a form suitable for antigen-loading of itDCs, such as naïve itDCs. In embodiments, the processing comprises obtaining a cell suspension, a cell lysate, a cell homogenate, cell exosomes, cell debris, conditioned medium, or a partially purified protein preparation. In other embodiments, the processing comprises obtaining proteins, protein fragments, fusion proteins, peptides, peptide mimeotypes, altered peptides, fusion peptides from the cells. In some embodiments, the processing includes an enrichment of cells from a cell population that displays a relevant antigen. In some embodiments, the enrichment results in a cell population that is at least 80%, at least 90%, at least 95%, at least 98%, at least 99% or 100% homogeneous in regard to an antigen of interest (i.e., the aforementioned percentages refer to the percent of cells in a population that express an antigen of interest). In some embodiments, the processing includes a purification of the cells, for example, from a mixed population of cells, or from a culture medium. In some embodiments, the processing comprises lysis of the cells to generate a crude cell lysate comprising antigen of interest. In some embodiments, the purification comprises fusing the cells to naïve itDCs, for example, by methods of electric pulse or chemical-induced cell fusion that are known to those of skill in the art. Additional methods of processing cells into a form suitable for uptake by itDCs are known to those of skill in the art and the invention is not limited in this respect.

“Circulating itDCs” refers to itDCs that are capable of circulating in the peripheral blood or migrating to one or more organs or tissues in a subject, or that are produced from dendritic cells that are capable of circulating in the peripheral blood or migrating to one or more organs or tissues in a subject. Examples of types of circulating itDCs include itDCs that are CD103+, CD11b+, XCR1+ or plasmacytoid itDCs. Another example of a type of circulating itDCs are those that are not CD8α+. Still another example of such itDCs are migratory itDCs. In other embodiments, the circulating itDCs are CD103+ itDCs. Methods and reagents for enrichment of DCs for particular subsets are known in the art. Non-limiting examples of such methods are sorting by fluorescence-activated cell sorting (FACS) and magnetic cell sorting (MACS). Both technologies involve binding agents, for example, antibodies, that bind to a surface marker characterisitic for a population of DCs of interest, for example, CD103, CD11b, XCR1, etc. and a separation step in which cells that bind to the binding agent are separated from cells that do not bind the binding agent. Populations of DC subsets can be isolated from various sources known to those of skill in the art, including, but not limited to, blood, e.g., peripheral blood or cord blood; lymphatic fluid; lymph nodes; bone marrow; thymus, liver or spleen.

The term “combining” refers to actively contacting one material, such as a population of cells with another material, such as another population of cells, or processed forms thereof, thus creating a mix or combination of materials, cell populations and/or processed forms. The term includes, in some embodiments, a combination under conditions that do not result in cell fusion. In other embodiments, the term includes contacting under conditions under which at least some of the cells of one population fuse with some of the cells of another population. Preferably, the combining of itDCs, or precursors thereof, with antigens of interest (provided in any of the forms provided herein) comprises contacting the itDCs, or precursors thereof, ex vivo.

“Concomitantly” means administering two or more substances to a subject in a manner that is correlated in time, preferably sufficiently correlated in time so as to provide a modulation in an immune response. In embodiments, concomitant administration may occur through administration of two or more substances in the same dosage form. In other embodiments, concomitant administration may encompass administration of two or more substances in different dosage forms, but within a specified period of time, preferably within 1 month, more preferably within 1 week, still more preferably within 1 day, and even more preferably within 1 hour.

“Dendritic cells,” also referred to herein as “DCs,” are antigen-presenting immune cells that process antigenic material and present it to other cells of the immune system, most notably to T cells. Immature DCs function to capture and process antigens. When DCs endocytose antigens, they process the antigens into smaller fragments, generally peptides, that are displayed on the DC surface, where they are presented to, for example, antigen-specific T cells through MHC molecules. After uptake of antigens, DCs migrate to the lymph nodes. Immature dendritic cells are characterized by high endocytic and micropinocytotic function. During maturation, DCs can be prompted by various signals, including signaling through Toll-like receptors (TLR), to express co-stimulatory signals that induce cognate effector T cells (Teff) to become activated and to proliferate, thereby initiating a T-cell mediated immune response to the antigen. Alternatively, DCs can present antigen to antigen-specific T cells without providing co-stimulatory signals (or while providing co-inhibitory signals), such that Teff are not properly activated. Such presentation can cause, for example, death or anergy of T cells recognizing the antigen, or can induce the generation and/or expansion of regulatory T cells (Treg). The term “dendritic cells” includes differentiated dendritic cells, immature, and mature dendritic cells. These cells can be characterized by expression of certain cell surface markers (e.g., CD11c, MHC class II, and at least low levels of CD80 and CD86), CD11b, CD304 (BDCA4)). In some embodiments, DCs express CD8, CD103, CD1d, etc. Other DCs can be identified by the absence of lineage markers such as CD3, CD14, CD19, CD56, etc. In addition, dendritic cells can be characterized functionally by their capacity to stimulate alloresponses and mixed lymphocyte reactions (MLR).

A “dendritic cell subset that possess a desired physiological characteristic” refers to a type of dendritic cell that possesses a particular physiological characteristic. Such physiological characteristics include, for example, the expression of one or more specific surface markers, the ability to present an antigen of interest in a specific context (e.g., MHC Class I or MHC Class II context), the ability to preferentially migrate to a specific organ or tissue of interest, the ability to promote anti-viral innate and/or adaptive immune responses, the ability to cross-present antigen, etc. In some embodiments, the subset is one that expresses XCR1 or CD103. In other embodiments, the term refers to a subset of plasmacytoid DCs.

There are a number of subtypes of tolerogenic dendritic cells in mammals, for example, and some aspects of this invention are based on the recognition that different types of induced tolerogenic dendritic cells can induce different types and/or levels of tolerogenic responses. As an example, XCR1+ DCs and plasmacytoid DCs (pDCs) mediate potent tolerogenic reaction upon administration to a subject, for example, a subject experiencing an undesired immune reaction to an antigen. Therefore, in some embodiments of the methods and compositions provided herein the subset of dendritic cells from which the itDCs, including antigen-specific itDCs, comprises XCR1+ DCs and pDCs. Such dendritic cells can be directly isolated from blood or differentiated in vitro as described herein.

As another example, certain subsets of DCs have trafficking or migrating properties to particular tissues, e.g., CD103+ DCs, DCs contacted with retinoic acid, as well as DCs obtained from the intestinal tract, which can preferentially migrate to the intestinal tract. CD103+ DCs that are TGF-β induced can also preferentially migrate to the intestinal tract. Generally, DCs obtained from certain tissues, including and in addition to DCs obtained from the intestinal tract, may migrate to the tissues from which they are obtained. Accordingly, a subset of DCs may be those obtained from a particular tissue. In embodiments, the subset of dendritic cells of the methods and compositions provided herein are those that migrate preferentially to a tissue of interest. One of the advantages of using specific subsets of DCs instead of mixed populations of DCs in the context of migrating to certain tissues is that the migration of the itDCs produced from such DCs to a target tissue is more efficient, thus achieving a desired tolerogenic effect at the target tissue with lower dosages of DCs. It follows, therefore, that such subsets can be used in subjects suffering from inflammatory disease, autoimmune disease, allergy or organ or tissue rejection, etc. where undesired immune processes occur at a particular organ or tissue site.

As another example, a subset of cells can be selected where certain antigen presentation is required. In embodiments, it may be desirable to induce a tolerogenic reaction to an antigen that comprises an MHC Class I-restricted epitope, but no MHC Class II-restricted epitope, and proper presentation will be restricted to DCs capable of cross-presenting the antigen in an MHC I context. In such embodiments, the subset can be dendritic cells that express the chemokine receptor XCR1. Accordingly in some embodiments of the methods and compositions provided where an antigen of interest is to be presented in an MHC Class I context, the use of a subset of XCR1+ DCs may be preferred as it can be more efficient as compared to the use of a mixed population of DCs.

Using the methods provided to produce itDCs from subsets of dendritic cells can provide dendritic cell subset-enriched itDCs. A composition comprising such itDCs are enriched for the subset of DCs, for example, for XCR1+, plasmacytoid DCs and/or CD103+ DCs, and this means that the compositions comprise more itDCs of a particular subset than expected or that the majority of the itDCs of a dendritic cell subset-enriched itDC population are of a particular subset. In some embodiments, all of the itDCs of the composition are of a particular subset. In other embodiments, when there is a heterogeneous population of itDCs, the number of itDCs of the particular subset is greater than the number of itDCs of each of the other subsets. In some embodiments, at least 10%, least 20%, least 30%, least 40%, least 50%, least 60%, least 70%, least 80%, least 90%, least 95%, least 97%, least 98%, least 99%, or least 99.9% of the total itDCs in a composition are of a particular subset.

Methods and reagents for enrichment of DCs for particular subsets are known in the art. Non-limiting examples of such methods are sorting by fluorescence-activated cell sorting (FACS) and magnetic cell sorting (MACS). Both technologies involve binding agents, for example, antibodies, that bind to a surface marker characterisitic for a population of DCs of interest, for example, CD103, CD11c or XCR1, and a separation step in which cells that bind to the binding agent are separated from cells that do not bind the binding agent. Populations of DC subsets can be isolated from various sources known to those of skill in the art, including, but not limited to, blood, e.g., peripheral blood or cord blood; lymphatic fluid; lymph nodes; bone marrow; thymus, liver or spleen.

“Derived” means prepared from a material or information related to a material but is not “obtained” from the material. Such materials may be substantially modified or processed forms of materials taken directly from a biological material. Such materials also include materials produced from information related to a biological material.

“Differentiated” cells are cells that have acquired a functional cell type and cannot or do not differentiate into another cell type. Examples of differentiated cells include, but are not limited to, β-cells, Tregs, Teffs, muscle cells, neurons, glial cells, and hepatocytes. Cells that are “pluripotent” are cells that have the potential to develop, or differentiate, into all fetal or adult cell types, but typically lack the potential to develop into placental cells. Non-limiting examples of pluripotent cells include embryonic stem cells and induced pluripotent stem (iPS) cells.

“Dosage form” means a pharmacologically and/or immunologically active material in a medium, carrier, vehicle, or device suitable for administration to a subject.

“Epitope”, also known as an antigenic determinant, is the part of an antigen that is recognized by the immune system, specifically by, for example, antibodies, B cells, or T cells. As used herein, “MHC Class I-restricted epitopes” are epitopes that are presented to immune cells by MHC class 1 molecules found on nucleated cells. “MHC Class II-restricted epitopes” are epitopes that are presented to immune cells by MHC class II molecules found on antigen presenting cells (APCs), for example, on professional antigen-presenting immune cells, such as on macrophages, B cells, and dendritic cells, or on non-hematopoietic cells, such as hepatocytes. “B cell epitopes” are molecular structures that are recognized by antibodies or B cells. In some embodiments, the epitope itself is an antigen.

A number of epitopes are known to those of skill in the art, and exemplary epitopes suitable according to some aspects of this invention include, but are not limited to those listed in the Immune Epitope Database (www.immuneepitope.org, Vita R, Zarebski L, Greenbaum J A, Emami H, Hoof I, Salimi N, Damle R, Sette A, Peters B. The immune epitope database 2.0. Nucleic Acids Res. 2010 Jan. 38(Database issue):D854-62; the entire contents of which as well as all database entries of IEDB version 2.4, August 2011, and particularly all epitopes disclosed therein, are incorporated herein by reference). Epitopes can also be identified with publicly available algorithms, for example, the algorithms described in Wang P, Sidney J, Kim Y, Sette A, Lund O, Nielsen M, Peters B. 2010. peptide binding predictions for HLA DR, DP and DQ molecules. BMC Bioinformatics 2010, 11:568; Wang P, Sidney J, Dow C, Mothé B, Sette A, Peters B. 2008. A systematic assessment of MHC class II peptide binding predictions and evaluation of a consensus approach. PLoS Comput Biol. 4(4):e1000048; Nielsen M, Lund O. 2009. NN-align. An artificial neural network-based alignment algorithm for MHC class II peptide binding prediction. BMC Bioinformatics. 10:296; Nielsen M, Lundegaard C, Lund O. 2007. Prediction of MHC class II binding affinity using SMM-align, a novel stabilization matrix alignment method. BMC Bioinformatics. 8:238; Bui H H, Sidney J, Peters B, Sathiamurthy M, Sinichi A, Purton K A, Mothé B R, Chisari F V, Watkins D I, Sette A. 2005. Immunogenetics. 57:304-314; Sturniolo T, Bono E, Ding J, Raddrizzani L, Tuereci O, Sahin U, Braxenthaler M, Gallazzi F, Protti M P, Sinigaglia F, Hammer J. 1999. Generation of tissue-specific and promiscuous HLA ligand databases using DNA microarrays and virtual HLA class II matrices. Nat. Biotechnol. 17(6):555-561; Nielsen M, Lundegaard C, Worning P, Lauemoller S L, Lamberth K, Buus S, Brunak S, Lund O. 2003. Reliable prediction of T-cell epitopes using neural networks with novel sequence representations. Protein Sci 12:1007-1017; Bui H H, Sidney J, Peters B, Sathiamurthy M, Sinichi A, Purton K A, Mothe B R, Chisari F V, Watkins D I, Sette A. 2005. Automated generation and evaluation of specific MHC binding predictive tools: ARB matrix applications. Immunogenetics 57:304-314; Peters B, Sette A. 2005. Generating quantitative models describing the sequence specificity of biological processes with the stabilized matrix method. BMC Bioinformatics 6:132; Chou P Y, Fasman G D. 1978. Prediction of the secondary structure of proteins from their amino acid sequence. Adv Enzymol Relat Areas Mol Biol 47:45-148; Emini E A, Hughes J V, Perlow D S, Boger J. 1985. Induction of hepatitis A virus-neutralizing antibody by a virus-specific synthetic peptide. J Virol 55:836-839; Karplus P A, Schulz G E. 1985. Prediction of chain flexibility in proteins. Naturwissenschaften 72:212-213; Kolaskar A S, Tongaonkar P C. 1990. A semi-empirical method for prediction of antigenic determinants on protein antigens. FEBS Lett276:172-174; Parker J M, Guo D, Hodges R S. 1986. New hydrophilicity scale derived from high-performance liquid chromatography peptide retention data: correlation of predicted surface residues with antigenicity and X-ray-derived accessible sites. Biochemistry 25:5425-5432; Larsen J E, Lund O, Nielsen M. 2006. Improved method for predicting linear B-cell epitopes. Immunome Res 2:2; Ponomarenko J V, Bourne P E. 2007. Antibody-protein interactions: benchmark datasets and prediction tools evaluation. BMC Struct Biol 7:64; Haste Andersen P, Nielsen M, Lund O. 2006. Prediction of residues in discontinuous B-cell epitopes using protein 3D structures. Protein Sci 15:2558-2567; Ponomarenko J V, Bui H, Li W, Fusseder N, Bourne P E, Sette A, Peters B. 2008. ElliPro: a new structure-based tool for the prediction of antibody epitopes. BMC Bioinformatics 9:514; Nielsen M, Lundegaard C, Blicher T, Peters B, Sette A, Justesen S, Buus S, and Lund O. 2008. PLoS Comput Biol. 4(7)e1000107. Quantitative predictions of peptide binding to any HLA-DR molecule of known sequence: NetMHCIIpan; the entire contents of each of which are incorporated herein by reference for disclosure of methods and algorithms for the identification of epitopes.

Other examples of epitopes that can be combined with or presented by the itDCs provided herein include any of the MHC Class I-restricted, MHC Class II-restricted and B cell epitopes as provided as SEQ ID NOs: 1-943. Without wishing to being bound by any particular theory, MHC Class I-restricted epitopes include those set forth in SEQ ID NOs: 1-186, MHC Class II-restricted epitopes include those set forth in SEQ ID NOs: 187-537, and B cell epitopes include those set forth in SEQ ID NOs: 538-943. These epitopes include MHC Class I-restricted autoantigens, MHC Class II-restricted epitopes of allergens and B cell epitopes of autoantigens and allergens.

“Generating” means causing an action, such as an immune response (e.g., a tolerogenic immune response) to occur, either directly oneself or indirectly, such as, but not limited to, an unrelated third party that takes an action through reliance on one's words or deeds.

“Identifying” is any action or set of actions that allows a clinician to recognize a subject as one who may benefit from the methods and compositions provided herein. Preferably, the identified subject is one who is in need of a tolerogenic immune response as provided herein. The action or set of actions may be either directly oneself or indirectly, such as, but not limited to, an unrelated third party that takes an action through reliance on one's words or deeds.

“Induced tolerogenic DCs” refers to dendritic cells capable of suppressing immune responses or generating tolerogenic immune responses, such as antigen-specific T cell-mediated immune responses, e.g., by reducing effector T cell responses to specific antigens, by effecting an increase in the number of antigen-specific regulatory T cells, etc. Induced tolerogenic DCs an be characterized by antigen specific tolerogenic immune response induction ex vivo and/or in vivo. Such induction refers to an induction of tolerogenic immune responses to one or more antigens of interest presented by the induced tolerogenic dendritic cells. In embodiments, induced tolerogenic dendritic cells have a tolerogenic phenotype that is characterized by at least one, if not all, of the following properties i) capable of converting naïve T cells to Foxp3+ T regulatory cells ex vivo and/or in vivo (e.g., inducing expression of FoxP3 in the naïve T cells); ii) capable of deleting effector T cells ex vivo and/or in vivo; iii) retain their tolerogenic phenotype upon stimulation with at least one TLR agonist ex vivo (and, in some embodiments, increase expression of costimulatory molecules in response to such stimulus); and/or iv) do not transiently increase their oxygen consumption rate upon stimulation with at least one TLR agonist ex vivo.

Starting populations of cells comprising dendritic cells and/or dendritic cell precursors may be “induced” by treatment, for example, ex vivo to become tolerogenic. In some embodiments, starting populations of dendritic cells or dendritic cell precursors are differentiated into dendritic cells prior to, as part of, or after induction, for example using methods known in the art that employ cytokines and/or maturation factors. In some embodiments, induced dendritic cells comprise fully differentiated dendritic cells. In some embodiments, induced dendritic cells comprise both immature and mature dendritic cells. In some embodiments, induced dendritic cells are enriched for mature dendritic cells.

“Inflammatory disease” means any disease, disorder or condition in which undesired inflammation occurs.

“Load” refers to the amount of antigen combined with the dendritic cells and taken up and/or presented, preferably on their surface. Dendritic cells can be loaded with antigen according to methods described herein. In some embodiments, it is desirable to assess the level of antigen-loading achieved. For example, in some embodiments, it is desirable, to confirm that loading is sufficient to achieve a tolerogenic immune response in a subject. In some embodiments, the tolerogenic immune response is a certain level of antigen-specific CD4+ T cell, CD8+ T cell or B cell proliferation and/or activity. In other embodiments, the tolerogenic immune response is a certain level of antigen-specific antibody production. In other embodiments, the tolerogenic immune response is a certainly level of regulatory cell production and/or activity. In yet other embodiments, the tolerogenic immune response is a certain level of regulatory (e.g., anti-inflammatory) cytokine production. Antigen-loading of dendritic cells can be assessed, for example, by assessing whether a population of itDCs is able to induce a tolerogenic response in vitro, for example, when contacted with non-adherent peripheral blood mononuclear cells (PBMCs). In some embodiments, the itDCs are contacted with a regulatory T cell (Treg) precursor population, or a population of cells comprising such a precursor, under conditions and for a time sufficient to induce activation and/or proliferation of the Treg cells. In some embodiments, the presence and/or the number or frequency of the Treg cells is measured after a time sufficient for induction and/or proliferation, for example, with an ELISPOT assay, which allows for single-cell detection. Alternatively, the presence or the number of Treg cells can be determined indirectly, for example, by measuring a molecule secreted by the Treg cells, or a cytokine specific for activation of Treg cells. In some embodiments, the presence of Treg cells in the cell population contacted with the itDCs indicates that antigen-loading is sufficient. In some embodiments, the number of Treg cells measured is compared to a control or reference number, for example, the number of antigen-specific Treg cells present or expected to be present in a sample not contacted with the itDCs or contacted with naïve DCs. In some embodiments, if the number of Treg cells in the cell population contacted with the itDCs is statistically significantly higher than the control or reference number, the antigen-loading of the itDCs is indicated to be sufficient. In embodiments, the load is a function of the amount of Treg cells generated as compared to one or more reference or control numbers. In other embodiment, the load is a function of the amount of antigen combined with the itDCs in addition to in addition to the activity observed and/or one or more reference or control numbers.

“Maintenance dose” refers to a dose that is administered to a subject, after an initial dose has resulted in an immunosuppressive (e.g., tolerogenic) response in a subject, to sustain a desired immunosuppressive (e.g., tolerogenic) response. A maintenance dose, for example, can be one that maintains the tolerogenic effect achieved after the initial dose, prevents an undesired immune response in the subject, or prevents the subject becoming a subject at risk of experiencing an undesired immune response, including an undesired level of an immune response. In some embodiments, the maintenance dose is one that is sufficient to sustain an appropriate level of antigen-specific T cell number and/or activity, antigen-specific antibody production, etc.

“MHC” refers to major histocompatibility complex, a large genomic region or gene family found in most vertebrates that encodes MHC molecules that display fragments or epitopes of processed proteins on the cell surface. The presentation of MHC:peptide on cell surfaces allows for surveillance by immune cells, usually a T cell. There are two general classes of MHC molecules: Class I and Class II. Generally, Class I MHC molecules are found on nucleated cells and present peptides to cytotoxic T cells. Class II MHC molecules are found on certain immune cells, chiefly macrophages, B cells and dendritic cells, collectively known as professional APCs. The best-known genes in the MHC region are the subset that encodes antigen-presenting proteins on the cell surface. In humans, these genes are referred to as human leukocyte antigen (HLA) genes.

“Obtained” means taken directly from a material and used with substantially no modification and/or processing.

“Pharmaceutically acceptable excipient” means a pharmacologically inactive material used together with the itDCs, including antigen-specific itDCs, to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Protocol” refers to any dosing regimen of one or more substances to a subject. A dosing regimen may include the amount, frequency and/or mode of administration. In some embodiments, such a protocol may be used to administer one or more compositions of the invention to one or more test subjects. Immune responses in these test subject can then be assessed to determine whether or not the protocol was effective in reducing an undesired immune response or generating a desired immune response (e.g., the promotion of a tolerogenic effect). Any other therapeutic and/or prophylactic effect may also be assessed instead of or in addition to the aforementioned immune responses. Whether or not a protocol had a desired effect can be determined using any of the methods provided herein or otherwise known in the art. For example, a population of cells may be obtained from a subject to which a composition provided herein has been administered according to a specific protocol in order to determine whether or not specific immune cells, cytokines, antibodies, etc. were reduced, generated, activated, etc. Useful methods for detecting the presence and/or number of immune cells include, but are not limited to, flow cytometric methods (e.g., FACS) and immunohistochemistry methods. Antibodies and other binding agents for specific staining of immune cell markers, are commercially available. Such kits typically include staining reagents for multiple antigens that allow for FACS-based detection, separation and/or quantitation of a desired cell population from a heterogeneous population of cells.

“Providing a subject” is any action or set of actions that causes a clinician to come in contact with a subject and administer a composition provided herein thereto or to perform a method provided herein thereupon. Preferably, the subject is one who is in need of a tolerogenic immune response as provided herein. The action or set of actions may be either directly oneself or indirectly, such as, but not limited to, an unrelated third party that takes an action through reliance on one's words or deeds.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Substantially no B cell epitopes” refers to the absence of B cell epitopes in an amount (by itself, within the context of the antigen, in conjunction with a carrier or in conjunction with an inventive composition) that stimulates substantial activation of a B cell response. In embodiments, a composition with substantially no B cell epitopes does not contain a measurable amount of B cell epitopes of an antigen. In other embodiments, such a composition may comprise a measurable amount of B cell epitopes of an antigen but said amount is not effective to generate a measurable B cell immune response (by itself, within the context of the antigen, in conjunction with a carrier or in conjunction with an inventive composition), such as antigen-specific antibody production or antigen-specific B cell proliferation and/or activity, or is not effective to generate a significant measurable B cell immune response (by itself, within the context of the antigen, in conjunction with a carrier or in conjunction with an inventive composition). In some embodiments, a significant measurable B cell immune response is one that produces or would be expected to produce an adverse clinical result in a subject. In other embodiments, a significant measurable B cell immune response is one that is greater than the level of the same type of immune response (e.g., antigen-specific antibody production or antigen-specific B cell proliferation and/or activity) produced by a control antigen (e.g., one known not to comprise B cell epitopes of the antigen or to stimulate B cell immune responses). In some embodiments, a significant measurable B cell immune response, such as a measurement of antibody titers (e.g., by ELISA) is 2-fold, 3-fold, 4-fold, 5-fold, 6-fold, 7-fold, 8-fold, 9-fold, 10-fold, 15-fold, 20-fold or more greater than the same type of response produced by a control (e.g., control antigen). In other embodiments, a composition with substantially no B cell epitopes is one that produces little to no antigen-specific antibody titers (by itself, within the context of the antigen, in conjunction with a carrier or in conjunction with an inventive composition). Such compositions include those that produce an antibody titer (as an EC50 value) of less than 500, 400, 300, 200, 100, 50, 40, 30, 20 or 10. In other embodiments, a significant measurable B cell immune response, is a measurement of the number or proliferation of B cells that is 10%, 25%, 50%, 100%, 2-fold, 3-fold, 4-fold, 5-fold, 6-fold, 7-fold, 8-fold, 9-fold, 10-fold, 15-fold, 20-fold or more greater that the same type of response produced by a control. Other methods for measuring B cell responses are known to those of ordinary skill in the art.

In embodiments, to ensure that a composition comprises substantially no B cell epitopes, antigens are selected such that they do not comprise B cell epitopes for loading onto the itDCs, or precursors thereof, as provided herein. In other embodiments, to ensure that a composition comprises substantially no B cell epitopes of an antigen, the itDCs, or precursors thereof, are produced and tested for B cell immune responses (e.g., antigen-specific antibody production, B cell proliferation and/or activity). Compositions that exhibit the desired properties may then be selected.

“T cell antigen” means a CD4+ T-cell antigen or CD8+ cell antigen. “CD4+ T-cell antigen” means any antigen that is recognized by and triggers an immune response in a CD4+ T-cell e.g., an antigen that is specifically recognized by a T-cell receptor on a CD4+ T cell via presentation of the antigen or portion thereof bound to a Class II major histocompatability complex molecule (MHC). “CD8+ T cell antigen” means any antigen that is recognized by and triggers an immune response in a CD8+ T-cell e.g., an antigen that is specifically recognized by a T-cell receptor on a CD8+ T cell via presentation of the antigen or portion thereof bound to a Class I major histocompatability complex molecule (MHC). In some embodiments, an antigen that is a T cell antigen is also a B cell antigen. In other embodiments, the T cell antigen is not also a B cell antigen. T cell antigens generally are proteins or peptides.

A “therapeutic protein” refers to any protein that may be administered to a subject and have a therapeutic effect. Such therapies include protein replacement and protein supplementation therapies. Such therapies also include the administration of exogenous or foreign protein, antibody therapies, and cell or cell-based therapies. Therapeutic proteins include enzymes, enzyme cofactors, hormones, blood clotting factors, cytokines, growth factors, monoclonal antibodies and polyclonal antibodies. Examples of other therapeutic proteins are provided elsewhere herein. Therapeutic proteins may be produced in, on or by cells and may be obtained from such cells or combined and/or administered in the form of such cells. In embodiments, the therapeutic protein is produced in, on or by mammalian cells, insect cells, yeast cells, bacteria cells, plant cells, transgenic animal cells, transgenic plant cells, etc. The therapeutic protein may be recombinantly produced in such cells. The therapeutic protein may be produced in, on or by a virally transformed cell. The therapeutic protein may also be produced in, on or by autologous cells that have been transfected, transduced or otherwise manipulated to express it. Alternatively, the therapeutic protein may be combined with the itDCs and/or administered as a nucleic acid or by introducing a nucleic acid into a virus, VLP, liposome, etc. and combining and/or administering such forms. Alternatively, the therapeutic protein may be obtained from such forms and combined and/or administered as the therapeutic protein itself. Subjects, therefore, include any subject that has received, is receiving or will receive any of the foregoing. Such subject includes subjects that have received, is receiving or will receive gene therapy, autologous cells that have been transfected, transduced or otherwise manipulated to express a therapeutic protein, polypeptide or peptide; or cells that express a therapeutic protein, polypeptide or peptide.

“Therapeutic protein antigen” means an antigen that is associated with a therapeutic protein, or a portion of which, that can be presented for recognition by cells of the immune system and that can generate an undesired immune response (e.g., the production of therapeutic protein-specific antibodies) against the therapeutic protein. Therapeutic protein antigens generally include proteins, polypeptides, peptides, lipoproteins, or are contained or expressed in, on or by cells.

“Tolerogenic immune response” means any immune response that can lead to immune suppression specific to an antigen or a cell, tissue, organ, etc. that expresses such an antigen. Such immune responses include any reduction, delay or inhibition in an undesired immune response specific to the antigen or cell, tissue, organ, etc. that expresses such antigen. Such immune responses also include any stimulation, production, induction, promotion or recruitment in a desired immune response specific to the antigen or cell, tissue, organ, etc. that expresses such antigen. Tolerogenic immune responses, therefore, include the absence of or reduction in an undesired immune response to an antigen that can be mediated by antigen reactive cells as well as the presence or promotion of suppressive cells. Tolerogenic immune responses as provided herein include immunological tolerance. To “generate a tolerogenic immune response” refers to the generation of any of the foregoing immune responses specific to an antigen or cell, tissue, organ, etc. that expresses such antigen. The tolerogenic immune response can be the result of MHC Class I-restricted presentation and/or MHC Class II-restricted presentation and/or B cell presentation and/or presentation by CD1d, etc.

Tolerogenic immune responses include any reduction, delay or inhibition in CD4+ T cell, CD8+ T cell or B cell proliferation and/or activity. Tolerogenic immune responses also include a reduction in antigen-specific antibody production. Tolerogenic immune responses can also include any response that leads to the stimulation, induction, production or recruitment of regulatory cells, such as CD4+ Treg cells, CD8+ Treg cells, Breg cells, etc. In some embodiments, the tolerogenic immune response, is one that results in the conversion to a regulatory phenotype characterized by the production, induction, stimulation or recruitment of regulatory cells.

Tolerogenic immune responses also include any response that leads to the stimulation, production or recruitment of CD4+ Treg cells and/or CD8+ Treg cells. CD4+ Treg cells can express the transcription factor FoxP3 and inhibit inflammatory responses and auto-immune inflammatory diseases (Human regulatory T cells in autoimmune diseases. Cvetanovich G L, Hafler D A. Curr Opin Immunol. 2010 December; 22(6):753-60. Regulatory T cells and autoimmunity. Vila J, Isaacs J D, Anderson A E. Curr Opin Hematol. 2009 July; 16(4):274-9). Such cells also suppress T-cell help to B-cells and induce tolerance to both self and foreign antigens (Therapeutic approaches to allergy and autoimmunity based on FoxP3+ regulatory T-cell activation and expansion. Miyara M, Wing K, Sakaguchi S. J Allergy Clin Immunol. 2009 April; 123(4):749-55). CD4+ Treg cells recognize antigen when presented by Class II proteins on APCs. CD8+ Treg cells, which recognize antigen presented by Class I (and Qa-1), can also suppress T-cell help to B-cells and result in activation of antigen-specific suppression inducing tolerance to both self and foreign antigens. Disruption of the interaction of Qa-1 with CD8+ Treg cells has been shown to dysregulate immune responses and results in the development of auto-antibody formation and an auto-immune lethal systemic-lupus-erythematosus (Kim et al., Nature. 2010 Sep. 16, 467 (7313): 328-32). CD8+ Treg cells have also been shown to inhibit models of autoimmune inflammatory diseases including rheumatoid arthritis and colitis (CD4+ CD25+ regulatory T cells in autoimmune arthritis. Oh S, Rankin A L, Caton A J. Immunol. Rev. 2010 January; 233(1):97-111. Regulatory T cells in inflammatory bowel disease. Boden E K, Snapper SB. Curr Opin Gastroenterol. 2008 November; 24(6):733-41). In some embodiments, the compositions provided can effectively result in both types of responses (CD4+ Treg and CD8+ Treg). In other embodiments, FoxP3 can be induced in other immune cells, such as macrophages, iNKT cells, etc., the compositions provided herein can result in one or more of these responses as well.

Tolerogenic immune responses also include, but are not limited to, the induction of regulatory cytokines, such as Treg cytokines; induction of inhibitory cytokines; the inhibition of inflammatory cytokines (e.g., IL-4, IL-1b, IL-5, TNF-α, IL-6, GM-CSF, IFN-γ, IL-2, IL-9, IL-12, IL-17, IL-18, IL-21, IL-22, IL-23, M-CSF, C reactive protein, acute phase protein, chemokines (e.g., MCP-1, RANTES, MIP-1α, MIP-1β, MIG, ITAC or IP-10), the production of anti-inflammatory cytokines (e.g., IL-4, IL-13, IL-10, etc.), chemokines (e.g., CCL-2, CXCL8), proteases (e.g., MMP-3, MMP-9), leukotrienes (e.g., CysLT-1, CysLT-2), prostaglandins (e.g., PGE2) or histamines; the inhibition of polarization to a Th17, Th1 or Th2 immune response; the inhibition of effector cell-specific cytokines: Th17 (e.g., IL-17, IL-25), Th1 (IFN-γ), Th2 (e.g., IL-4, IL-13); the inhibition of Th1-, Th2- or Th17-specific transcription factors; the inhibition of proliferation of effector T cells; the induction of apoptosis of effector T cells; the induction of tolerogenic dendritic cell-specific genes; the induction of FoxP3 expression; the inhibition of IgE induction or IgE-mediated immune responses; the inhibition of antibody responses (e.g., antigen-specific antibody production); the inhibition of T helper cell response; the production of TGF-β and/or IL-10; the inhibition of effector function of autoantibodies (e.g., inhibition in the depletion of cells, cell or tissue damage or complement activation); etc.

Any of the foregoing may be measured in vivo in one or more animal models or may be measured in vitro. One of ordinary skill in the art is familiar with such in vivo or in vitro measurements. Undesired immune responses or tolerogenic immune responses can be monitored using, for example, methods of assessing immune cell number and/or function, tetramer analysis, ELISPOT, flow cytometry-based analysis of cytokine expression, cytokine secretion, cytokine expression profiling, gene expression profiling, protein expression profiling, analysis of cell surface markers, PCR-based detection of immune cell receptor gene usage (see T. Clay et al., “Assays for Monitoring Cellular Immune Response to Active Immunotherapy of Cancer” Clinical Cancer Research 7:1127-1135 (2001)), etc. Undesired immune responses or tolerogenic immune responses may also be monitored using, for example, methods of assessing protein levels in plasma or serum, T cell or B cell proliferation and functional assays, etc. In some embodiments, tolerogenic immune responses can be monitored by assessing the induction of FoxP3. In addition, specific methods are described in more detail in the Examples.

Preferably, tolerogenic immune responses lead to the inhibition of the development, progression or pathology of the diseases, disorders or conditions described herein. Whether or not the inventive compositions can lead to the inhibition of the development, progression or pathology of the diseases, disorders or conditions described herein can be measured with animal models of such diseases, disorders or conditions. In some embodiments, the reduction of an undesired immune response or generation of a tolerogenic immune response may be assessed by determining clinical endpoints, clinical efficacy, clinical symptoms, disease biomarkers and/or clinical scores. Undesired immune responses or tolerogenic immune responses can also be assessed with diagnostic tests to assess the presence or absence of a disease, disorder or condition as provided herein. Undesired immune responses can further be assessed by methods of measuring therapeutic proteins levels and/or function in a subject. In embodiments, methods for monitoring or assessing undesired allergic responses include assessing an allergic response in a subject by skin reactivity and/or allergen-specific antibody production.

In some embodiments, monitoring or assessing the generation of an undesired immune response or a tolerogenic immune response in a subject can be prior to the administration of a composition of itDCs, including antigen-specific itDCs, provided herein and/or prior to administration of a therapeutic protein or transplantable graft or exposure to an allergen. In other embodiments, assessing the generation of an undesired immune response or tolerogenic immune response can be after administration of a composition of itDCs provided herein and/or and after administration of a therapeutic protein or transplantable graft or exposure to an allergen. In some embodiments, the assessment is done after administration of the composition of itDCs, but prior to administration of the therapeutic protein or transplantable graft or exposure to an allergen. In other embodiments, the assessment is done after administration of the therapeutic protein or transplantable graft or exposure to an allergen, but prior to administration of the composition. In still other embodiments, the assessment is performed prior to both the administration of the itDCs and the therapeutic protein or transplantable graft or exposure to an allergen, while in yet other embodiments the assessment is performed after administration of both the itDCs and the therapeutic protein or transplantable graft or exposure to an allergen. In further embodiments, the assessment is performed both prior to and after the administration of the itDCs and/or the therapeutic protein or transplantable graft or exposure to an allergen. In still other embodiments, the assessment is performed more than once on the subject to determine that a desirable immune state is maintained in the subject, such as a subject that has or is at risk of having an inflammatory disease, an autoimmune disease, an allergy, organ or tissue rejection or graft versus host disease. Other subjects include those that have undergone or will undergo transplantation as well as those that have received, are receiving or will receive a therapeutic protein against which they have experienced, are experiencing or are expected to experience an undesired immune response.

An antibody response can be assessed by determining one or more antibody titers. “Antibody titer” means a measurable level of antibody production. Methods for measuring antibody titers are known in the art and include Enzyme-linked Immunosorbent Assay (ELISA). In embodiments, the antibody response can be quantitated, for example, as the number of antibodies, concentration of antibodies or titer. The values can be absolute or they can be relative. Assays for quantifying an antibody response include antibody capture assays, enzyme-linked immunosorbent assays (ELISAs), inhibition liquid phase absorption assays (ILPAAs), rocket immunoelectrophoresis (RIE) assays and line immunoelectrophoresis (LIE) assays. When an antibody response is compared to another antibody response the same type of quantitative value (e.g., titer) and method of measurement (e.g., ELISA) is preferably used to make the comparison.

An ELISA method for measuring an antibody titer, for example, a typical sandwich ELISA, may consist of the following steps (i) preparing an ELISA-plate coating material such that the antibody target of interest is coupled to a substrate polymer or other suitable material (ii) preparing the coating material in an aqueous solution (such as PBS) and delivering the coating material solution to the wells of a multiwell plate for overnight deposition of the coating onto the multiwell plate (iii) thoroughly washing the multiwell plate with wash buffer (such as 0.05% Tween-20 in PBS) to remove excess coating material (iv) blocking the plate for nonspecific binding by applying a diluent solution (such as 10% fetal bovine serum in PBS), (v) washing the blocking/diluent solution from the plate with wash buffer (vi) diluting the serum sample(s) containing antibodies and appropriate standards (positive controls) with diluent as required to obtain a concentration that suitably saturates the ELISA response (vii) serially diluting the plasma samples on the multiwell plate such to cover a range of concentrations suitable for generating an ELISA response curve (viii) incubating the plate to provide for antibody-target binding (ix) washing the plate with wash buffer to remove antibodies not bound to antigen (x) adding an appropriate concentration of a secondary detection antibody in same diluent such as a biotin-coupled detection antibody capable of binding the primary antibody (xi) incubating the plate with the applied detection antibody, followed by washing with wash buffer (xii) adding an enzyme such as streptavidin-HRP (horse radish peroxidase) that will bind to biotin found on biotinylated antibodies and incubating (xiii) washing the multiwell plate (xiv) adding substrate(s) (such as TMB solution) to the plate (xv) applying a stop solution (such as 2N sulfuric acid) when color development is complete (xvi) reading optical density of the plate wells at a specific wavelength for the substrate (450 nm with subtraction of readings at 570 nm) (xvi) applying a suitable multiparameter curve fit to the data and defining half-maximal effective concentration (EC50) as the concentration on the curve at which half the maximum OD value for the plate standards is achieved.

A “transplantable graft” refers to a biological material, such as cells, tissues and organs (in whole or in part) that can be administered to a subject. Transplantable grafts may be autografts, allografts, or xenografts of, for example, a biological material such as an organ, tissue, skin, bone, nerves, tendon, neurons, blood vessels, fat, cornea, pluripotent cells, differentiated cells (obtained or derived in vivo or in vitro), etc. In some embodiments, a transplantable graft is formed, for example, from cartilage, bone, extracellular matrix, or collagen matrices. Transplantable grafts may also be single cells, suspensions of cells and cells in tissues and organs that can be transplanted. Transplantable cells typically have a therapeutic function, for example, a function that is lacking or diminished in a recipient subject. Some non-limiting examples of transplantable cells are β-cells, hepatocytes, hematopoietic stem cells, neuronal stem cells, neurons, glial cells, or myelinating cells. Transplantable cells can be cells that are unmodified, for example, cells obtained from a donor subject and usable in transplantation without any genetic or epigenetic modifications. In other embodiments, transplantable cells can be modified cells, for example, cells obtained from a subject having a genetic defect, in which the genetic defect has been corrected, or cells that are derived from reprogrammed cells, for example, differentiated cells derived from cells obtained from a subject.

“Transplantation” refers to the process of transferring (moving) a transplantable graft into a recipient subject (e.g., from a donor subject, from an in vitro source (e.g., differentiated autologous or heterologous native or induced pluripotent cells)) and/or from one bodily location to another bodily location in the same subject.

“Undesired immune response” refers to any undesired immune response that results from exposure to an antigen, promotes or exacerbates a disease, disorder or condition provided herein (or a symptom thereof), or is symptomatic of a disease, disorder or condition provided herein, etc. Such immune responses generally have a negative impact on a subject's health or is symptomatic of a negative impact on a subject's health.

### C. INVENTIVE COMPOSITIONS

Provided herein are methods and compositions and dosage forms related to induced tolerogenic dendritic cells produced from cells of a dendritic cell subset. Producing itDCs from such subsets allows itDCs to be produced that are enriched in the cells of a particular subset and, accordingly, the physiological characteristics provided by the cells of a particular subset. Thus, tolerogenic responses can be fine-tuned by using cells of a particular subset in the production of itDCs, which can subsequently be administered to a subject. Such itDCs are useful for the suppression, inhibition, prevention, or delay of the onset of an undesired immune response in a subject, as described in more detail elsewhere herein. Such subjects include those that have or are at risk of having an inflammatory disease, an autoimmune disease, an allergy, organ or tissue rejection or graft versus host disease. Such subjects also include those that have been, are being or will be administered a therapeutic protein against which the subject has experienced or is expected to experience an undesired immune response. Such subjects also include those that have undergone or will undergo transplantation.

The induced tolerogenic dendritic cells for use in the compositions and methods provided have a tolerogenic phenotype that is characterized by, for example, at least one of the following properties i) capable of converting naïve T cells to Foxp3+ T regulatory cells ex vivo and in vivo; ii) capable of deleting effector T cells ex vivo and in vivo; iii) retain their tolerogenic phenotype upon stimulation with at least one TLR agonist ex vivo (and in some embodiments, increase expression of costimulatory molecules with the same stimulus); and/or iv) do not transiently increase their oxygen consumption rate upon stimulation with at least one TLR agonist ex vivo. In some embodiments, the itDCs have at least 2 of the above properties. In some embodiments, the itDCs have at least 3 of the above properties. In yet some embodiments, the itDCs have all 4 of the above properties. Induced tolerogenic DCs that convert naïve T cells to Foxp3+ T regulatory cells are itDCs that induce expression of the transcription factor Foxp3 in naïve T cells, e.g., in the absence of cell division, such that naïve T cells that did not previously express Foxp3 are induced to express Foxp3 and become T reg cells. In addition to expression of Foxp3, T regulatory cells (Treg cells) express CD25 and are capable of sustained suppression of effector T cell responses.

It is known in the art that stimulation of Toll-like receptors (TLR) on the surface of DCs promotes DC activation, allowing DCs to induce proliferation of effector T cells. However, the itDCs described herein for use in the compositions and methods provided maintain their tolerogenic phenotype (are tolerogenically locked) even after being contacted with a maturation stimulus ex vivo, e.g., after stimulation with at least one TLR agonist. The presence of the tolerogenic phenotype of the cells can be demonstrated functionally, e.g., by confirming that cells treated with a maturation stimulus retain their functional tolerogenic phenotype as described herein. In some embodiments, induced tolerogenic dendritic cells treated with a maturation stimulus increase expression of costimulatory molecules (as compared to the level of expression of costimulatory molecules prior to stimulation), but retain their tolerogenic phenotype. Exemplary costimulatory molecules include one or more of CD80, CD86, and ICOS ligand. In some embodiments, induced tolerogenic dendritic cells treated with a maturation stimulus increase their expression of class II molecules and/or migratory capacities (as compared to the level of expression of class II molecules prior to stimulation), but retain their tolerogenic phenotype. Tolerogenically locked itDCs may be produced by a tolerogenic locking protocol in which dendritic cells or dendritic cell precursors are treated in an ex vivo environment with a tolerogenic locking agent which renders them capable of, for example, at least one of: i) converting naïve T cells to Foxp3+ T regulatory cells ex vivo and ii) deleting effector T cells ex vivo. Further methods of producing tolerogenically locked itDCs are described in more detail below.

In embodiments, the antigens that are presented by the antigen-specific itDCs are combined with the itDCs, or precursors thereof, in the presence of an agent that enhances the uptake, processing or presentation of antigens. Preferably, the loading of an antigen on the itDCs of the compositions and methods provided will lead to a tolerogenic immune response against the antigen and/or the cells in, by or on which the antigen is expressed. The antigens include any of the antigens provided herein. Such antigens include antigens associated with an inflammatory disease, autoimmune disease, allergy, organ or tissue rejection, graft versus host disease, a transplantable graft and a therapeutic protein or portion thereof.

Therapeutic proteins include, but are not limited to, infusible therapeutic proteins, enzymes, enzyme cofactors, hormones, blood clotting factors, cytokines and interferons, growth factors, monoclonal antibodies, and polyclonal antibodies (e.g., that are administered to a subject as a replacement therapy), and proteins associated with Pompe's disease (e.g., alglucosidase alfa, rhGAA (e.g., Myozyme and Lumizyme (Genzyme)). Therapeutic proteins also include proteins involved in the blood coagulation cascade. Therapeutic proteins include, but are not limited to, Factor VIII, Factor VII, Factor IX, Factor V, von Willebrand Factor, von Heldebrant Factor, tissue plasminogen activator, insulin, growth hormone, erythropoietin alfa, VEGF, thrombopoietin, lysozyme, antithrombin and the like. Therapeutic proteins also include adipokines, such as leptin and adiponectin. Other examples of therapeutic proteins are as described below and elsewhere herein. Also included are fragments or derivatives of any of the therapeutic proteins provided as the epitope, or protein, polypeptide or peptide that comprises the epitope.

Examples of therapeutic proteins used in enzyme replacement therapy of subjects having a lysosomal storage disorder include, but are not limited to, imiglucerase for the treatment of Gaucher's disease (e.g., CEREZYME™), a-galactosidase A (a-gal A) for the treatment of Fabry disease (e.g., agalsidase beta, FABRYZYME™), acid a-glucosidase (GAA) for the treatment of Pompe disease (e.g., alglucosidase alfa, LUMIZYME™, MYOZYME™), arylsulfatase B for the treatment of Mucopolysaccharidoses (e.g., laronidase, ALDURAZYME™, idursulfase, ELAPRASE™, arylsulfatase B, NAGLAZYME™).

Examples of enzymes include oxidoreductases, transferases, hydrolases, lyases, isomerases, and ligases.

Examples of hormones include Melatonin (N-acetyl-5-methoxytryptamine), Serotonin, Thyroxine (or tetraiodothyronine) (a thyroid hormone), Triiodothyronine (a thyroid hormone), Epinephrine (or adrenaline), Norepinephrine (or noradrenaline), Dopamine (or prolactin inhibiting hormone), Antimullerian hormone (or mullerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Calcitonin, Cholecystokinin, Corticotropin-releasing hormone, Erythropoietin, Follicle-stimulating hormone, Gastrin, Ghrelin, Glucagon, Glucagon-like peptide (GLP-1), GIP, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Luteinizing hormone, Melanocyte stimulating hormone, Orexin, Oxytocin, Parathyroid hormone, Prolactin, Relaxin, Secretin, Somatostatin, Thrombopoietin, Thyroid-stimulating hormone (or thyrotropin), Thyrotropin-releasing hormone, Cortisol, Aldosterone, Testosterone, Dehydroepiandrosterone, Androstenedione, Dihydrotestosterone, Estradiol, Estrone, Estriol, Progesterone, Calcitriol (1,25-dihydroxyvitamin D3), Calcidiol (25-hydroxyvitamin D3), Prostaglandins, Leukotrienes, Prostacyclin, Thromboxane, Prolactin releasing hormone, Lipotropin, Brain natriuretic peptide, Neuropeptide Y, Histamine, Endothelin, Pancreatic polypeptide, Renin, and Enkephalin.

Examples of blood and blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, prekallikrein (Fletcher factor), high-molecular weight kininogen (HMWK) (Fitzgerald factor), fibronectin, fibrin, thrombin, antithrombin III, heparin cofactor II, protein C, protein S, protein Z, protein Z-related protease inhibitot (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit).

Examples of cytokines include lymphokines, interleukins, and chemokines, type 1 cytokines, such as IFN-γ, TGF-β, and type 2 cytokines, such as IL-4, IL-10, and IL-13.

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumour_necrosis_factor-alpha(TNF-α), Vascular endothelial growth factor (VEGF), Wnt Signaling Pathway, placental growth factor (P1GF), [(Foetal Bovine Somatotrophin)] (FBS), IL-1, IL-2, IL-3, IL-4, IL-5, IL-6, and IL-7.

Examples of monoclonal antibodies include Abagovomab, Abciximab, Adalimumab, Adecatumumab, Afelimomab, Afutuzumab, Alacizumab pegol, ALD, Alemtuzumab, Altumomab pentetate, Anatumomab mafenatox, Anrukinzumab, Anti-thymocyte globin, Apolizumab, Arcitumomab, Aselizumab, Atlizumab (tocilizumab), Atorolimumab, Bapineuzumab, Basiliximab, Bavituximab, Bectumomab, Belimumab, Benralizumab, Bertilimumab, Besilesomab, Bevacizumab, Biciromab, Bivatuzumab mertansine, Blinatumomab, Brentuximab vedotin, Briakinumab, Canakinumab, Cantuzumab mertansine, Capromab pendetide, Catumaxomab, Cedelizumab, Certolizumab pegol, Cetuximab, Citatuzumab bogatox, Cixutumumab, Clenoliximab, Clivatuzumab tetraxetan, Conatumumab, Dacetuzumab, Daclizumab, Daratumumab, Denosumab, Detumomab, Dorlimomab aritox, Dorlixizumab, Ecromeximab, Eculizumab, Edobacomab, Edrecolomab, Efalizumab, Efungumab, Elotuzumab, Elsilimomab, Enlimomab pegol, Epitumomab cituxetan, Epratuzumab, Erlizumab, Ertumaxomab, Etaracizumab, Exbivirumab, Fanolesomab, Faralimomab, Farletuzumab, Felvizumab, Fezakinumab, Figitumumab, Fontolizumab, Foravirumab, Fresolimumab, Galiximab, Gantenerumab, Gavilimomab, Gemtuzumab ozogamicin, GC1008, Girentuximab, Glembatumumab vedotin, Golimumab, Gomiliximab, Ibalizumab, Ibritumomab tiuxetan, Igovomab, Imciromab, Infliximab, Intetumumab, Inolimomab, Inotuzumab ozogamicin, Ipilimumab, Iratumumab, Keliximab, Labetuzumab, Lebrikizumab, Lemalesomab, Lerdelimumab, Lexatumumab, Libivirumab, Lintuzumab, Lorvotuzumab mertansine, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab-CD3, Nacolomab tafenatox, Naptumomab estafenatox, Natalizumab, Nebacumab, Necitumumab, Nerelimomab, Nimotuzumab, Nofetumomab merpentan, Ocrelizumab, Odulimomab, Ofatumumab, Olaratumab, Omalizumab, Oportuzumab monatox, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, Rafivirumab, Ramucirumab, Ranibizumab, Raxibacumab, Regavirumab Reslizumab, Rilotumumab, Rituximab, Robatumumab, Rontalizumab, Rovelizumab, Ruplizumab, Satumomab pendetide, Sevirumab, Sibrotuzumab, Sifalimumab, Siltuximab, Siplizumab, Solanezumab, Sonepcizumab, Sontuzumab, Stamulumab, Sulesomab, Tacatuzumab tetraxetan, Tadocizumab, Talizumab, Tanezumab, Taplitumomab paptox, Tefibazumab, Telimomab aritox, Tenatumomab, Teneliximab, Teplizumab, Ticilimumab (tremelimumab), Tigatuzumab, Tocilizumab (atlizumab), Toralizumab, Tositumomab, Trastuzumab, Tremelimumab, Tucotuzumab celmoleukin, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab aritox.

Examples of infusion therapy or injectable therapeutic proteins include, for example, Tocilizumab (Roche/Actemra®), alpha-1 antitrypsin(Kamada/AAT), Hematide® (Affymax and Takeda, synthetic peptide), albinterferon alfa-2b (Novartis/Zalbin™), Rhucin® (Pharming Group, C1 inhibitor replacement therapy), tesamorelin (Theratechnologies/Egrifta, synthetic growth hormone-releasing factor), ocrelizumab (Genentech, Roche and Biogen), belimumab (GlaxoSmithKline/Benlysta®), pegloticase (Savient Pharmaceuticals/Krystexxa™), taliglucerase alfa (Protalix/Uplyso), agalsidase alfa (Shire/Replagal®), velaglucerase alfa (Shire).

Additional therapeutic proteins useful in accordance to aspects of this invention will be apparent to those of skill in the art, and the invention is not limited in this respect.

In some embodiments, the itDCs, including the antigen-specific itDCs, are combined with a transplantable graft or therapeutic protein, and such compositions are provided herein. In other embodiments, the itDCs are administered prior to, concomitantly with or after the administration of a transplantable graft, therapeutic protein, etc.

In some embodiments, the composition of the invention are formulated as a dosage form. Appropriate carriers or vehicles for administration (e.g., for pharmaceutical administration) of cells are compatible with cell viability and are known in the art. Such carriers may optionally include buffering agents or supplements that promote cell viability. In some embodiments, cells to be administered are formulated with one or more additional agents, e.g., survival enhancing factors or pharmaceutical agents. In some embodiments, cells are formulated with a liquid carrier which is compatible with survival of the cells.

Compositions according to the invention, therefore, may further comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, the compositions are suspended in sterile saline solution for injection together with a preservative.

Typical inventive compositions may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

In some embodiments, a cell, antigen, etc., may be isolated. Isolated refers to the element being separated from its native environment and present in sufficient quantities to permit its identification or use. This means, for example, the element may be (i) selectively produced by expression cloning or (ii) purified as by chromatography or electrophoresis. Isolated elements may be, but need not be, substantially pure. Because an isolated element may be admixed with a pharmaceutically acceptable excipient in a pharmaceutical preparation, the element may comprise only a small percentage by weight of the preparation. The element is nonetheless isolated in that it has been separated from the substances with which it may be associated in living systems, i.e., isolated from other lipids or proteins. Any of the elements provided herein may be isolated. Any of the antigens provided herein can be included in the compositions in isolated form.

### D. METHODS OF MAKING AND USING THE INVENTIVE COMPOSITIONS

Some aspects of this invention provide methods of generating itDCs, including antigen-specific itDCs, and related compositions, and some aspects provide methods of using the itDCs provided herein. The itDCs, including the antigen-specific itDCs, may be produced by the methods provided herein. The antigen-specific itDCs may also be produced according to the methods provided in the PCT Publication, WO2011/109833.

In one embodiment, a protocol for producing itDCs for use in the methods provided employs one or more respirostatic agents for treatment of dendritic cells of a particular dendritic cell subset or dendritic cell precursors that may be differentiated to dendritic cells of a particular subset ex vivo to produce induced tolerogenic DCs capable of antigen specific tolerance induction by, for example, i) converting naïve T cells into FoxpP3+ CD4+ regulatory T cells, and/or ii) deleting effector T cells. In another embodiment, a protocol employs at least one agent which tolerogenically locks dendritic cells of a particular dendritic cell subset or dendritic cell precursors that may be differentiated to dendritic cells of a particular subset ex vivo to produce induced tolerogenic DCs capable of antigen specific tolerance induction by, for example, i) converting naïve T cells into FoxpP3+ CD4+ regulatory T cells, and/or ii) deleting effector T cells.

In some embodiments, itDCs are generated by treating a starting population of cells comprising dendritic cells of a particular dendritic cell subset and/or dendritic cell precursors that may be differentiated to dendritic cells of a particular subset with a tolerogenic stimulus. To obtain starting cell populations, samples of cells, tissues, or organs comprising dendritic cells of a particular dendritic cell subset and/or dendritic cell precursors that may be differentiated to dendritic cells of a particular subset are isolated from a subject, e.g., a human subject, using methods known in the art.

In some embodiments, a starting population which comprises dendritic cells of a particular dendritic cell subset and/or dendritic cell precursors that may be differentiated to dendritic cells of a particular subset is derived from splenic tissue. In some embodiments, a starting cell population which comprises dendritic cells of a particular dendritic cell subset and/or dendritic cell precursors that may be differentiated to dendritic cells of a particular subset is derived from thymic tissue. In some embodiments, a starting cell population is derived from bone marrow. In some embodiments, a starting cell population is derived from peripheral blood, e.g., from whole blood or from a sub-population obtained from blood, for example, via leukopheresis.

In some embodiments, a starting population of cells comprises dendritic cell precursors that may be differentiated to dendritic cells of a particular subset. In some embodiments, a population of cells comprising dendritic cell precursors can be harvested from the peripheral blood using standard mononuclear cell leukopheresis, a technique that is well known in the art. Dendritic cell precursors can then be collected, e.g., using sequential buoyant density centrifugation steps. For example, the leukopheresis product can be layered over a buoyant density solution (specific gravity=1.077 g/mL) and centrifuged at 1,000 g for 20 minutes to deplete erythrocytes and granulocytes. The interface cells are collected, washed, layered over a second buoyant density solution (specific gravity=1.065 g/mL), and centrifuged at 805 g for 30 minutes to deplete platelets and low-density monocytes and lymphocytes. The resulting cell pellet is enriched for dendritic cell precursors. Alternatively, a kit, such as EasySep Human Myeloid DC Enrichment Kit, designed to isolate dendritic cells from fresh blood or ammonium chloride-lysed leukophoresis by negative selection may also be used.

In some embodiments, a starting population of cells comprising dendritic cells can be obtained using methods known in the art. Such a population may comprise myeloid dendritic cells (mDC), plasmacytoid dendritic cells (pDC), and/or dendritic cells generated in culture from monocytes (e.g., MO-DC, MDDC). In some embodiments, dendritic cells and/or dendritic cell precursors can also be derived from a mixed cell population containing such cells (e.g., from the circulation or from a tissue or organ) and then selecting the subset cells of interest. In certain embodiments, the mixed cell population containing DC and/or dendritic cell precursors is enriched such that cells of a particular subset or cells of interest make up greater than 50% (e.g., 55%, 60%, 65%, 70%, 75%, 80%, 85%, 90%, 95%, 98%, 99%, 99.5%, 99.9% or more) of the cell population. In some embodiments, the dendritic cells described herein are purified by separation from some or all non-dendritic cells in a cell population. In exemplary embodiments, cells can be purified such that a starting population contains at least 50% or more dendritic cells of a particular dendritic cell subset and/or dendritic cell precursors that may be differentiated to dendritic cells of a particular subset, e.g., a purity of 50%, 55%, 60%, 65%, 70%, 75%, 80%, 85%, 90%, 95%, 98%, 99%, 99.5%, 99.9% or more.

In some embodiments, the cells can be isolated using the techniques described in Current Protocols in Immunology, Wiley Interscience, Nov. 19, 2009, or in Woo et al., Transplantation, 58:484 (1994), the entire contents of which are incorporated herein by reference. Those skilled in the art are able to implement modifications to the foregoing methods of isolating cells comprising dendritic cells of a particular dendritic cell subset or dendritic cell precursors that may be differentiated to dendritic cells of a particular subset without the exercise of undue experimentation. In some embodiments, dendritic cells can be purified using fluorescence-activated cell sorting for antigens present on their surface, e.g., CD11c in the case of certain dendritic cells. In some embodiments, DCs present in a starting population of cells express CD11c. In some embodiments, DCs and/or dendritic cell precursors present in a starting population of cells express class II molecules. A starting population of cells may be monitored for expression of various cell surface markers (e.g., including CD11c) using techniques known in the art. Other markers that can be used to select the cells of interest are provided elsewhere herein or are otherwise known to those of ordinary skill in the art.

In some embodiments, a population of cells comprising dendritic cells and/or dendritic cell precursors can be obtained from pluripotential cells present in blood as PBMCs. Although most easily obtainable from blood, the pluripotential cells may also be obtained from any tissue in which they reside, including bone marrow and spleen tissue. These pluripotential cells typically express CD14, CD32, CD68 and CD115 monocyte markers with little or no expression of CD83, p55 or accessory molecules such as CD40 and CD86.

In some embodiments, dendritic cell precursors can be differentiated into dendritic cells of a particular subset using methods known in the art prior to, during, or after treatment with at least one agent in a protocol to prepare induced tolerogenic dendritic cells. For example, when cultured in the presence of cytokines such as a combination of GM-CSF and IL-4 or IL-13, the pluripotential cells give rise to the immature dendritic cells. In some embodiments, FLT3 Ligand can be used for this purpose. For example, in some embodiments, a starting population of cells comprising dendritic cells and/or dendritic cell precursors can be cultured ex vivo in the presence of one or more agents which promote differentiation of DCs. In some embodiments, one or more of GMCSF or IL-4 is used to promote the development of DCs ex vivo, e.g., by culture for 1-15 days, 2-10 days, 3-9 days, 4-8 days, or 5-6 days or such other time to obtain sufficient differentiation. In some embodiments, induced dendritic cells are fully differentiated (either prior to, during, or after induction to produce induced tolerogenic dendritic cells).

In some embodiments, a starting population of cells can be obtained from PBMCs. Methods of obtaining PBMCs from blood, using methods such as differential sedimentation through an appropriate medium, e.g. Ficoll-Hypaque [Pharmacia Biotech, Uppsala, Sweden], are well known and suitable for use in this invention. In a preferred embodiment of the invention, the pluripotential cells are obtained by depleting populations of PBMCs of platelets, and T and B lymphocytes. Various methods may be used to accomplish the depletion of the non-pluripotential cells. According to one method, immunomagnetic beads labeled with antibodies specific for cells to be removed, e.g., T and/or B lymphocytes, either directly or indirectly may be used to remove the T and B cells from the PBMC population. T cells may also be depleted from the PBMC population by rosetting with neuramimidase treated red blood cells as described by O'Dherty (1993), which is incorporated herein by reference. In some embodiments, to produce 3 million mature dendritic cells, approximately 40 mls of blood can be processed. In some embodiments, 4 to 8×10⁷ pluripotential PBMC give rise to approximately 3 million mature dendritic cells.

Cultures of immature dendritic cells may be obtained by culturing the pluripotent cells in the presence of cytokines which promote their differentiation for a time sufficient to achieve the desired level of differentiation, e.g., from 1-10 days, from 2-9 days, from 3-8 days, or from 4-7 days. As an example, a combination of GM-CSF and IL-4 at a concentration of each at between about 200 to about 2000 U/ml, between about 500 and 1000 U/ml, or about 800 U/ml (GM-CSF) and 1000 U/ml (IL-4) produces significant quantities of the immature dendritic cells. A combination of GM-CSF (10-200 ng/ml) and IL-4 (5-50 ng/ml) can also be used. It may also be desirable to vary the concentration of cytokines at different stages of the culture such that freshly cultured cells are cultured in the presence of higher concentrations of IL-4 (1000 U/ml) than established cultures (500 U/ml IL-4 after 2 days in culture). Other cytokines such as IL-13 may be found to substitute for IL-4. In some embodiments, FLT3 ligand can be used for this purpose. Other protocols for this purpose are known in the art.

Methods for obtaining these immature dendritic cells from adherent blood mononuclear fractions are described in Romani et al. (1994); and Sallusto and Lanzavecchia, 1994) both of which are incorporated herein by reference. Briefly, lymphocyte depleted PBMCs are plated in tissue culture plates at a density of about 1 million cells/cm2 in complete culture medium containing cytokines such as GM-CSF and IL-4 at concentrations of each at between about 800 to 1000 U/ml and IL-4 is present at about 1000 U/ml.

In some embodiments, the source of immature dendritic cells is a culture of proliferating dendritic cell precursors prepared according to a method described in Steinman et al. International application PCT/US93/03141, which is incorporated herein by reference. Since the dendritic cells prepared from the CD34+ proliferating precursors mature to dendritic cells expressing mature characteristics it is likely that they also pass through a development stage where they are pluripotent.

In some embodiments, a starting population of cells can be enriched for the presence of mature dendritic cells by contacting the immature dendritic cells with a dendritic cell maturation factor. As referred to herein, the dendritic cell maturation factor may actually be one or more specific substances which act alone or with another agent to cause the maturation of the immature dendritic cells, for example, with one or more of an adjuvant, a TLR agonist, a CD40 agonist, an inflammasome activator, an inflammatory cytokine, or combinations thereof.

The tolerogenic stimuli includes substances which, alone or in combination, induce a dendritic cell of a particular dendritic cell subset or dendritic cell precursor that may be differentiated to dendritic cells of a particular subset to become tolerogenic, e.g., by inducing the dendritic cell to become capable of increasing the proportion of antigen specific Treg cells to antigen specific Teff cells in a cell population. More specifically, induced tolerogenic dendritic cells are produced by one or more agents which induce a tolerogenic phenotype in the DCs characterized by, for example, at least one of the following properties i) induced tolerogenic DCs are capable of converting naïve T cells to Foxp3+ T regulatory cells ex vivo and in vivo; ii) induced tolerogenic DCs are capable of deleting effector T cells ex vivo and in vivo; iii) induced tolerogenic DCs retain their tolerogenic phenotype upon stimulation with at least one TLR agonist ex vivo (while in some embodiments, they increase expression of costimulatory molecules); and/or iv) induced tolerogenic DCs do not transiently increase their oxygen consumption rate upon stimulation with at least one TLR agonist ex vivo.

Exemplary tolerogenic stimuli include those agents which do not increase mitochondrial activation (e.g., as measured by oxygen consumption) or which disrupt electron transport in cells. Other exemplary tolerogenic stimuli include those agents which tolerogenically lock induced DCs into a tolerogenic phenotype. Exemplary tolerogenic stimuli include agents include inhibitors of mammalian Target of Rapamycin (mTOR), agonists of TGFβ pathway signaling, statins, purinergic receptor pathway antagonists, and agents which inhibit mitochondrial electron transport, either alone or in combination. In some embodiments, a tolerogenic stimulus does not consist of rapamycin alone. In some embodiments, a tolerogenic stimulus does not consist of an mTOR inhibitor alone.

In some embodiments, after treatment with one or more tolerogenic stimuli (such as those set forth below, known in the art, or identified using the methods described herein) the cells may be removed from the agents, e.g., by centrifugation and/or by washing prior to further manipulation.

Exemplary agents that can constitute a tolerogenic stimulus include, but are not limited to mTOR inhibitors, TGFβ pathway agonists, statins, purinergic receptor pathway agonists, and certain agents disrupting electron transport. It should be appreciated that additional tolerogenic stimuli, for example, additional agents that can constitute a tolerogenic stimulus, are known to those of skill in the art, and that the invention is not limited in this respect.

For example, in some embodiments, the invention provides methods of producing a population of cells comprising induced tolerogenic DCs, wherein the method comprises contacting a starting population of cells comprising dendritic cells or dendritic cell precursors ex vivo with a tolerogenic stimulus. In some embodiments, the tolerogenic stimulus comprises at least one agent that promotes the induction of tolerogenic dendritic cells, or that results in the emergence of itDCs in the cell population. In some embodiments, the at least one agent is selected from the group consisting of: i) an mTOR inhibitor and a TGFβ agonist; ii) a statin; iii) an mTOR inhibitor and a statin; iv) an mTOR inhibitor, a TGFβ agonist, and a statin; v) a purinergic receptor antagonist; vi) a purinergic receptor antagonist and a statin; vii) a purinergic receptor antagonist and an mTOR inhibitor; viii) a purinergic receptor antagonist, an mTOR inhibitor and a TGFβ agonist; ix) a purinergic receptor antagonist, an mTOR inhibitor, a TGFβ agonist and a statin; x) an agent which disrupts mitochondrial electron transport in the DCs; xi) an agent which disrupts mitochondrial electron transport in the DCs and an mTOR inhibitor; xii) an agent which disrupts mitochondrial electron transport in the DCs and a statin; xiii) an agent which disrupts mitochondrial electron transport in the DCs, an mTOR inhibitor, and a TGFβ agonist; and xiv) an agent which disrupts mitochondrial electron transport in the DCs, an mTOR inhibitor, a TGFβ agonist, and a statin.

In some embodiments, the at least one agent is selected from the group consisting of: i) an mTOR inhibitor and a TGFβ agonist; ii) a statin; iii) an mTOR inhibitor, a TGFβ agonist, and a statin; iv) a purinergic receptor antagonist; and v) an agent which disrupts mitochondrial electron transport in the DCs.

In some embodiments, the at least one agent is a respirostatic agent or an agent that promotes respirostatic tolerance.

In some embodiments, the at least one agent comprises an mTOR inhibitor and a TGFβ agonist. In some embodiments, the mTOR inhibitor comprises rapamycin or a derivative or analog thereof. In some embodiments, the TGFβ agonist is selected from the group consisting of TGFβ1, TGFβ2, TGFβ3, and mixtures thereof. In some embodiments, the at least one agent comprises a purinergic receptor antagonist. In some embodiments, the purinergic receptor antagonist binds to a purinergic receptor selected from the group consisting of P1, P2X, P2X7, and P2Y. In some embodiments, the purinergic receptor antagonist is oxidized ATP.

In some embodiments, the starting population of cells comprising dendritic cells of a particular dendritic cell subset or dendritic cell precursors that may be differentiated to dendritic cells of a particular subset is contacted with the at least one agent for a period of time sufficient for the induction of tolerogenic dendritic cells, or the emergence of such cells in the population. In some embodiments, the starting population of cells is contacted with the at least one agent for less than 10 h. In some embodiments, the starting population of cells is contacted with the at least one agent for about 30 min, about 1 h, about 2 h, about 3 h, about 4 h, about 5 h, about 6 h, about 7 h, about 8 h, or about 9 h. In some embodiments, the starting population of cells is contacted with the at least one agent for about 1-3 h, for example, for 2 h. In some embodiments, the starting population of cells is contacted with a composition comprising at least one agent selected from the group consisting of: a purinergic receptor antagonist, an mTOR inhibitor, a TGFβ receptor antagonist, a statin, an agent which disrupts mitochondrial electron transport in the DCs for less than 10 h.

Some exemplary agents that constitute a tolerogenic stimulus are described in more detail below:

1. mTOR Inhibitors

In some exemplary embodiments, a tolerogenic stimulus for use in the instant invention comprises or consists of an mTOR inhibitor. mTOR inhibitors suitable for practicing the invention include inhibitors or antagonists of mTOR or mTOR-induced signaling. mTOR inhibitors include rapamycin and analogs, portions, or derivatives thereof, e.g., Temsirolimus (CCI-779), everolimus (RAD001) and deforolimus (AP23573). Additional rapamycin derivatives include 42- and/or 31-esters and ethers of rapamycin, which are disclosed in the following patents, all hereby incorporated by reference in their entirety: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers are disclosed in the patents listed above. 27-esters and ethers of rapamycin are disclosed in U.S. Pat. No. 5,256,790, which is hereby incorporated by reference in its entirety. Oximes, hydrazones, and hydroxylamines of rapamycin are disclosed in U.S. Pat. Nos. 5,373,014, 5,378,836, 5,023,264, and 5,563,145, which are hereby incorporated by reference in their entirety. The preparation of these oximes, hydrazones, and hydroxylamines are disclosed in the foregoing patents. The preparation of 42-oxorapamycin is disclosed in U.S. Pat. No. 5,023,263, which is hereby incorporated by reference in its entirety.

Other mTOR inhibitors include PI-103, XL765, Torin1, PP242, PP30, NVP-BEZ235, and OSI-027. Additional mTOR inhibitors include LY294002 and wortmannin. Other inhibitors of mTOR are described in U.S. Pat. Nos. 7,504,397 and 7,659,274, and in Patent Publication Nos. US20090304692A1; US20090099174A1, US20060199803A1, WO2008148074A3, the entire contents of which are incorporated herein by reference. In some embodiments, an mTOR inhibitor (e.g., rapamycin or a variant or derivative thereof) is used in combination with one or more statins. In some embodiments, an mTOR inhibitor (e.g., rapamycin or a variant or derivative thereof) is used in combination with a TGFβ pathway agonist.

2. TGFβ Pathway Agonists

In some exemplary embodiments, a tolerogenic stimulus for use in the instant invention comprises or consists of one or more TGFβ agonists. TGFβ agonists suitable for practicing the invention include substances that stimulate or potentiate responses induced by TGFβ signaling. In some embodiments, a TGFβ pathway agonist is acts by modulating TGFβ receptor-mediated signaling. In some embodiments, a TGFβ pathway agonist is a TGFβ mimetic, e.g., a small molecule having TGFβ-like activity (e.g., biaryl hydroxamates, A-161906 as described in Glaser et al. 2002. Molecular Cancer Therapeutics 1:759-768, or other histone deacetylase inhibitors (such as spiruchostatins A and B or diheteropeptin).

In exemplary embodiments, a TGFβ receptor agonist useful for practicing the invention is TGFβ, including TGFβ1, TGFβ2, TGFβ3, variants thereof, and mixtures thereof. Additional TGFβ agonists are described in Patent Publication No. US20090143394A1, the entire contents of which are incorporated herein by reference.

In particular embodiments, the foregoing TGFβ agonists are used in the presence of an mTOR inhibitor for producing induced tolerogenic DC.

3. Statins

Statins are HMG-CoA reductase inhibitors, a class of drug used to lower cholesterol levels by inhibiting the enzyme HMG-CoA reductase, which plays a central role in the production of cholesterol in the liver. Exemplary statins include atorvastatin (Lipitor and Torvast), fluvastatin (Lescol), lovastatin (Mevacor, Altocor, Altoprev), pitavastatin (Livalo, Pitava), pravastatin (Pravachol, Selektine, Lipostat), rosuvastatin (Crestor), simvastatin (Zocor, Lipex). In some embodiments, at least one statin is used alone for producing induced tolerogenic dendritic cells. In some embodiments, at least one statin is used in combination with an mTOR inhibitor.

4. Purinergic Receptor Pathway Antagonists

In some exemplary embodiments, a tolerogenic stimulus for use in the instant invention comprises or consists of one or more purinergic agonists. Purinergic receptor pathway antagonists suitable for practicing the invention include inhibitors or antagonists of purinergic receptor activity or purinergic receptor signaling. Particular purinergic receptor antagonists include compounds that inhibit the activity of or signaling through the purinergic receptors P1, P2X, P2X7, and/or P2Y. These receptors bind extracellular adenosine triphosphate (ATP). In some embodiments, a purinergic receptor antagonist useful for practicing the invention is oxidized ATP (oATP).

In some embodiments, purinergic receptor antagonists useful for practicing the invention include one or more of the compounds described in the following U.S. patents, the entire contents of which are incorporated herein by reference: U.S. Pat. No. 7,235,549, U.S. Pat. No. 7,214,677, U.S. Pat. No. 7,553,972, U.S. Pat. No. 7,241,776, U.S. Pat. No. 7,186,742, U.S. Pat. No. 7,176,202, U.S. Pat. No. 6,974,812, U.S. Pat. No. 7,071,223, and U.S. Pat. No. 7,407,956. In some embodiments, purinergic receptor antagonists useful for practicing the invention include one or more of the compounds described in the following patent publications, the entire contents of which are incorporated herein by reference: WO2010018280A1, WO2008142194A1, WO2009074519A1, WO2008138876A1, WO2008119825A3, WO2008119825A2, WO2008125600A3, WO2008125600A2, WO06083214A1, WO03047515A3, WO03047515A2, WO03042191A1, WO2008119685A3, WO2008119685A2, WO06003517A1, WO04105798A1, WO2008116814A1, WO2007056046A1, WO2009132000A1, WO2009077559A3, WO2009077559A2, WO2009074518A1, WO2008003697A1, WO2007056091A3, WO2007056091A2, WO06136004A1, WO05111003A1, WO05019182A1, WO04105796A1, WO04073704A1, WO2009077362A1, US20070032465A1, WO2009053459A1, US20080009541A1, WO2007008157A1, WO2007008155A1, US20070105842A1, WO06017406A1, US20060058302A1, US20060018904A1, WO05025571A1, WO04105797A1, WO04099146A1, WO04058731A1, WO04058270A1, US20030186981A1, WO2009057827A1, US20080171733A1, WO2007002139C1, WO2007115192A3, WO2007115192A2, WO2007002139A3, WO2007002139A2, US20070259920A1, US20070049584A1, WO06086229A1, US20060247257A1, US20060052374A1, WO05014555A1, US20090220516A1, US20090042886A1, US20080207577A1, US20070281939A1, US20070281931A1, US20070249666A1, US20070232686A1, US20070142329A1, US20070122849A1, US20070082930A1, US20070010497A1, US20060217430A1, US20060211739A1, US20060040939A1, US20060025614A1, US20050009900A1, and US20040180894A1.

In particular embodiments, purinergic receptor antagonists useful for practicing the invention include one or more of oATP, suranim, clopidogrel, prasugrel, ticlopidine, ticagrelor, A740003, A438079, pyridoxalphosphate-6-azophenyl-2′,4′-disulfonic acid (PPADS), pyridoxal 5′-phosphate (P5P), periodate-oxidized ATP, 5-(N,N-hexamethylene)amiloride (HMA), KN62 (1-[N,O-bis(5-isoquinolinesulfonyl)-N-methyl-L-tyrosyl]-4-phenylpiperazine), suramin, 2.Chloro-5-[[2-(2-hydroxy-ethylamino)-ethylamino]-methyl]-N-(tricyclo[3.3.1.13,7]dec-1-ylmethyl)-benzamide, 2.Chloro-5-[3-[(3-hydroxypropyl)amino]propyl]-N-(tricyclo[3.3.1.1]dec-1-ylmethyl)-benzamide, (R)-2-Chloro-5-[3-[(2-hydroxy-1-methylethyl)amino]propyl]-N-(tricyclo[3.3.1.13,7]dec-1-ylmethyl)-benzamide, 2.Chloro-5-[[2-[(2-hydroxyethyl)amino]ethoxy]methyl]-N-(tricyclo[3.3.1.13,7]dec-1-ylmethyl)-benzamide, 2.Chloro-5-[3-[3-(methylamino)propoxy]propyl]-N-(tricyclo[3.3.1.13,7]dec-1-ylmethyl)benzamide, 2.Chloro-5-[3-(3-hydroxy-propylamino)-propoxy]-N-(tricyclo[3.3.1.13,7]dec-1-ylmethyl)-benzamide, 2.Chloro-5-[2-(3-hydroxypropylamino)ethylamino]-N-(tricyclo[3.3.1.13,7]dec-1-ylmethyl)-benzamide, 2.Chloro-5-[2-(3-hydroxypropylsulfonyl)ethoxy]-N-(tricyclo[3.3.1.13,7]dec-1-ylmethyl)-benzamide, 2.Chloro-5-[2-[2-[(2-hydroxyethyl)amino]ethoxy]ethoxy]-N-(tricyclo[3.3.1.13,7]dec-1-ylmethyl)-benzamide, 2.Chloro-5-[[2-[[2-(1-methyl-1H-imidazol-4-yl)ethyl]amino]ethyl]amino]-N-(tricyclo[3.3.1.13,7]dec-1-ylmethyl)-benzamide, 2.Chloro-5-piperazin-1-ylmethyl-N-(tricyclo[3.3.1.1]dec-1-ylmethyl)-benzamide, 2.Chloro-5-(4-piperidinyloxy)-N-(tricyclo[3.3.1.13,7]dec-1-ylmethyl)-benzamide, 2.Chloro-5-(2,5-diazabicyclo[2.2.1]hept-2-ylmethyl)-N-(tricyclo[3.3.1.1]dec-1-ylmethyl)-benzamide, 2.Chloro-5-(piperidin-4-ylsulfinyl)-N-(tricyclo[3.3.1.13,7]dec-1-ylmethyl)-benzamide, 5.Chloro-2-[3-[(3-hydroxypropyl)amino]propyl]-N-(tricyclo[3.3.1.13,7]dec-1-ylmethyl)-4-pyridinecarboxamide, 5.Chloro-2-[3-(ethylamino)propyl]-N-(tricyclo[3.3.1.13,7]dec-1-ylmethyl)-4-pyridinecarboxamide, 5.Chloro-2-[3-[(2-hydroxyethyl)amino]propyl]-N-(tricyclo[3.3.1.13,7]dec-1-ylmethyl)-4-pyridinecarboxamide, 5.Chloro-2-[3-[[(2S)-2-hydroxypropyl]amino]propyl]-N-(tricyclo[3.3.1.13,7]dec-1-ylmethyl)-4-pyridinecarboxamide, N-[2-Methyl-5-(9-oxa-3,7-diazabicyclo[3.3.1]non-3-ylcarbonyl)phenyl]-tricyclo[3.3.1.13,7]decane-1-acetamide, or combinations thereof.

5. Agents Which Disrupt Electron Transport

In some embodiments, an agent which disrupts electron transport can be used to induce tolerogenicity in dendritic cells. Such agents include, e.g., rotenone, antimycinA, and oligomycin.

6. Combinations of Agents

In some exemplary embodiments, the tolerogenic stimulus comprises or consists of a combination of agents, e.g., a cocktail of agents, for example, more than one of the agents set forth above. Exemplary tolerogenic stimuli include at least one respirostatic or tolerogenic locking agent which can be used to produce induced tolerogenic dendritic cells. In some embodiments, the at least one agent comprises an mTOR inhibitor and a TGFβ agonist. In some embodiments, the at least one agent comprises a statin. In some embodiments, the at least one agent comprises an mTOR inhibitor and a statin. In some embodiments, the at least one agent comprises an mTOR inhibitor, a TGFβ agonist, and a statin. In some embodiments, the at least one agent comprises a purinergic receptor antagonist. In some embodiments, the at least one agent comprises a purinergic receptor antagonist and a statin. In some embodiments, the at least one agent comprises a purinergic receptor antagonist and an mTOR inhibitor. In some embodiments, the at least one agent comprises a purinergic receptor antagonist, an mTOR inhibitor and a TGFβ agonist. In some embodiments, the at least one agent comprises a purinergic receptor antagonist, an mTOR inhibitor, a TGFβ agonist and a statin. In some embodiments, the at least one agent comprises an agent which disrupts mitochondrial electron transport in the DCs. In some embodiments, the at least one agent comprises an agent which disrupts mitochondrial electron transport in the DCs and an mTOR inhibitor. In some embodiments, the at least one agent comprises an agent which disrupts mitochondrial electron transport in the DCs and a statin. In some embodiments, the at least one agent comprises an agent which disrupts mitochondrial electron transport in the DCs, an mTOR inhibitor, and a TGFβ agonist. In some embodiments, the at least one agent comprises an agent which disrupts mitochondrial electron transport in the DCs, an mTOR inhibitor, a TGFβ agonist, and a statin.

In some exemplary embodiments, the tolerogenic stimulus comprises or consists of a combination of agents selected from the group consisting of: i) an mTOR inhibitor (e.g., rapamycin or a variant or derivative thereof); a TGFβ agonist (e.g., TGFβ; ii) a statin; an mTOR inhibitor (e.g., rapamycin or a variant or derivative thereof), a TGFβ agonist (e.g., TGFβ, and a statin; iv) a purinergic receptor antagonist (e.g., oATP); and v) an agent which disrupts mitochondrial electron transport in the DCs (e.g., rotenone).

7. Concentrations of Tolerogenic Stimuli

Exemplary concentrations of tolerogenic stimuli for producing induced tolerogenic cells can be readily determined by a person of skill in the art by titration of the stimulus on a starting population of cells in culture and testing the phenotype of the induced cells ex vivo. In some embodiments, a concentration of agent is chosen which has the desired effect on oxygen consumption rate (e.g., no change in the rate or a reduction in the rate) in dendritic cells. In some embodiments, a concentration of agent is chosen which has the desired effect on the induction of Treg cells. In exemplary embodiments, tolerogenic stimuli are used at a concentrations of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In some embodiments, tolerogenic stimuli are used at concentrations of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein.

In some embodiments, an mTOR inhibitor (e.g., rapamycin or a derivative or variant thereof) is used as a tolerogenic stimulus at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In exemplary embodiments, an mTOR inhibitor e.g., rapamycin is used at a concentration of 1 μM or 10 nM. In some embodiments, an mTOR inhibitor (e.g., rapamycin or a derivative or variant thereof) is used at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 5 μg/ml, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein.

In some embodiments, one or more statins are used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In some embodiments, a statin is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In some exemplary embodiments, a statin is used at a concentration of about 10, 30, 50, 75, 100, or 300 μM.

In some embodiments, a TGFβ agonist is used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 20 ng/ml, 30 ng/ml, 50 ng/ml, 75 ng/ml, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL and ranges therein. In some embodiments, a TGFβ agonist is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM. In exemplary embodiments, TGFβ is used as a tolerogenic stimulus at a concentration of 20 ng/mL.

In some embodiments, a purinergic receptor antagonist (e.g., oATP) is used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In some embodiments, a purinergic receptor antagonist is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein In exemplary embodiments, oATP is used as a tolerogenic stimulus at a concentration of 100 uM-1 mM.

In some embodiments, an agent which disrupts mitochondrial electron transport is used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In some embodiments, an agent which disrupts mitochondrial electron transport is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein.

In some embodiments, when combinations of agents are used, the concentration of each may be reduced.

8. Timing of Exposure

In general, exposure of a starting population of cells comprising dendritic cells of a particular dendritic cell subset and/or dendritic cell precursors that may be differentiated to dendritic cells of a particular subset to at least one tolerogenic stimulus is of a time sufficient to create induced tolerogenic dendritic cells, e.g., as demonstrated by a tolerogenic phenotype. In some embodiments, cells, for example, a starting population of cells are contacted with at least one tolerogenic stimulus for at least one hour. In some embodiments, cells are contacted with at least one tolerogenic stimulus for at least two hours. In some embodiments, cells are contacted with at least one tolerogenic stimulus for at least three hours. In some embodiments, cells are contacted with at least one tolerogenic stimulus for at least four hours. In some embodiments, cells are contacted with at least one tolerogenic stimulus for at least five hours. In some embodiments, cells are contacted with at least one tolerogenic stimulus for at least six hours. In some embodiments, cells are contacted with at least one tolerogenic stimulus for at least seven hours. In some embodiments, cells are contacted with at least one tolerogenic stimulus for at least eight hours. In some embodiments, cells are contacted with at least one tolerogenic stimulus for at least nine hours. In some embodiments, cells are contacted with at least one tolerogenic stimulus for at least ten hours. In some embodiments, cells are contacted with at least one tolerogenic stimulus for at least eleven hours. In some embodiments, cells are contacted with at least one tolerogenic stimulus for at least twelve hours. In some embodiments, cells are contacted with at least one tolerogenic stimulus for at least thirteen hours. In some embodiments, cells are contacted with at least one tolerogenic stimulus for at least fourteen hours. In some embodiments, cells are contacted with at least one tolerogenic stimulus for at least fifteen hours. In some embodiments, cells are contacted with at least one tolerogenic stimulus for at least sixteen hours.

In some embodiments, cells, for example, a starting population of cells are contacted with at least one tolerogenic stimulus for from one to seventy two hours, e.g., from two to forty eight hours, from three to twenty four hours, from four to sixteen hours, from five to twelve hours, from four to ten hours, from five to eight hours.

In some embodiments, cells, for example, a starting population of cells are contacted with at least one tolerogenic stimulus for at least one hour and less than ten hours. In some embodiments, cells are contacted with at least one tolerogenic stimulus for at least two hours and less than ten hours. In some embodiments, cells are contacted with at least one tolerogenic stimulus for at least three hours and less than ten hours. In some embodiments, cells are contacted with at least one tolerogenic stimulus for at least four hours and less than ten hours. In some embodiments, cells are contacted with at least one tolerogenic stimulus for at least five hours and less than ten hours. In some embodiments, cells are contacted with at least one tolerogenic stimulus for at least six hours and less than ten hours. In some embodiments, cells are contacted with at least one tolerogenic stimulus for at least seven hours and less than ten hours. Some such embodiments, which employ shorter incubation times than previously taught or suggested in the art are described in some, but not all of the appended Examples. In some embodiments, such shorter incubation times are employed for treatment of starting populations of cells comprising or enriched for fully differentiated dendritic cells (e.g., populations of cells which have been treated to differentiate dendritic cell precursors). In some embodiments, such shorter incubation times are employed for treatment of starting populations of cells comprising dendritic cell precursors (e.g., populations of cells which have not been treated to differentiate dendritic cell precursors). In some embodiments, shorter incubation time improves yields of viable cells and can be used for treatment of cells with mTOR inhibitors (e.g., rapamycin and variants or derivatives thereof) alone. In addition, these short incubation times can be used to produce tolerogenic dendritic cells using e.g., respirostatic or tolerogenic locking agents.

In some embodiments, mitochondrial respiration of cells can be tested to ensure that treatment with an inducing agent, for example, an agent that constitutes a tolerogenic stimulus, results in an appropriate response. For example, in some embodiments, O₂ consumption (the oxygen consumption rate; OCR) by cells can be measured. For example, induced tolerogenic dendritic cells can be tested to ensure that O₂ consumption decreases or does not increase. OCR can be measured, e.g., using an analyzer such as the Seahorse XF24 flux analyzer of Clark electrode. In some embodiments, a different assay can also be used to confirm the effect of an agent on mitochondrial function. For example, in some embodiments, mRNA levels of the expression of one or more of PGC-1a, PGC-1b, PRC, or other molecules involved in mitochondrial function, such as estrogen-related receptor α, NRF-1, NRF-2, Sp1, YY1, CREB and MEF-2/E-box factors can be measured. For example, induced tolerogenic dendritic cells exposed to a tolerogenic stimulus can be tested to ensure that levels of PGC-1a mRNA do not increase or decrease. Other methods of testing mitochondrial function which are known in the art can also be used for this purpose.

For example, alternative readouts of DC metabolism can be measured. For example, glucose uptake (e.g., using derivatized glucose) can be measured, as can the presence of reactive oxygen species (e.g., using DCF-DA). In some embodiments, lactic acid production (which is elevated with increased glycolysis and/or decreased mitochondrial activity) can be measured. In some embodiments, the extracellular acidification rate (ECAR) can be measured and is reflective of lactic acid production by glycolysis or pyruvate overload. The Seahorse SF24 flux analyzer can be used for this purpose. In yet some embodiments, cellular ATP/ADP ratios may be measured (e.g., using commercially available kits or as in Nagel et al. 2010. Methods Mol. Biol. 645:123-31). Increased levels of ATP and decreased levels of ADP have been recognized in proliferating cells and are a measure of activation.

In some embodiments, whether the induced tolerogenic dendritic cells have, for example, at least one of the following properties can be tested ex vivo using methods known in the art and/or described herein i) the ability to convert naïve T cells to Foxp3+ T regulatory cells ex vivo; ii) the ability to delete effector T cells ex vivo; iii) the ability to express costimulatory molecules but retain their tolerogenic phenotype upon stimulation with at least one TLR agonist ex vivo; and/or iv) the ability to remain respirostatic upon stimulation with at least one TLR agonist ex vivo.

To make the antigen-specific itDCs, the itDCs are contacted, or “loaded,” with the antigen of interest. Alternatively, precursors, such as dendritic cells before they are induced to have the tolerogenic phenotype as provided herein, can be loaded with the antigen of interest. These dendritic cells may then be further manipulated to form itDCs. ItDCs of the invention may express an antigen of interest intrinsically (e.g., the antigen may be an intrinsic antigen such as a germline gene product such as a self protein, polypeptide, or peptide), in which case they will not need to be further modified. For example, in some embodiments, where tolerance to an alloantigen is desired, itDCs which intrinsically express the alloantigen to which tolerance is desired, will not need to be manipulated to express an antigen of interest.

In some embodiments, dendritic cells which do not already express the antigen of interest such that it can be recognized by immune cells are made to express the antigen of interest or are contacted with the antigen of interest, e.g., by being bathed or cultured with the antigen, such that the dendritic cells will display the antigen on their surface for presentation (e.g., after processing or by directly binding to MHC).

In some embodiments, itDCs can be directly contacted with (e.g., bathed in or pulsed with) antigen. In other embodiments, the cells may express the antigen or may be engineered to express an antigen by transfecting the cells with an expression vector directing the expression of the antigen of interest such that the antigen is expressed and then displayed on the surface of the DCs. The antigen of interest may be provided in the form as elsewhere described herein, e.g., by contacting the itDCs with an antigen or a cell that expresses the antigen. Accordingly, in some embodiments, prior to, during, and/or following treatment with a tolerogenic stimulus, the cells are exposed to antigen. In some embodiments, before the cells have been induced with a tolerogenic stimulus, the cells are exposed to antigen. In some embodiments, after the cells have been induced with a tolerogenic stimulus, the cells are exposed to antigen. The antigen may be provided as a population of cells, processed forms thereof, a crude preparation comprising many proteins, polypeptides, and/or peptides (e.g., a lysate or extract) or may comprise one or more purified proteins, polypeptides, or peptides. Such proteins, polypeptides, or peptides can be naturally occurring, chemically synthesized, or expressed recombinantly.

For example, in some embodiments, cells are contacted with an antigen which is heterogeneous, e.g., which comprises more than one protein, polypeptide, or peptide. In some embodiments, such a protein antigen is a cell lysate, extract or other complex mixture of proteins. In some embodiments, an antigen with which cells are contacted comprises or consists of a protein which comprises a number of different immunogenic peptides. In some embodiments, the cells are contacted with the intact antigen and the antigen is processed by the cells. In some embodiments, the cells are contacted with purified components of the antigen, e.g., a mixture of immunogenic peptides, which may be further processed or may bind directly to MHC molecules on the cells.

In some embodiments, the cells are cultured in the presence of antigen for an appropriate amount of time (e.g., for 4 hours or overnight) under certain conditions (e.g., at 37° C.). In other embodiments, the cells are sonicated with antigen or the antigen is sonicated in buffer before loading.

In some embodiments, the antigen is targeted to surface receptors on DCs, e.g., by making antigen-antibody complexes (Fanger 1996), Ag-Ig fusion proteins (You et al. 2001) or heat shock protein-peptide constructs (Suzue K 1997, Arnold-Schild 1999, Todryk 1999). In some embodiments, non-specific targeting methods such as cationic liposome association with Ag (Ignatius 2000), apoptotic bodies from tumor cells (Rubartelli 1997, Albert 1998a, Albert 1998b), or cationic fusogenic peptides (Laus 2000) can be used.

In some embodiments, the antigen comprises or consists of a polypeptide that can be endocytosed, processed, and presented by dendritic cells. In some embodiments, the antigen comprises or consists of a short peptide that can be presented by dendritic cells without the need for processing. Short peptide antigens can bind to MHC class II molecules on the surface of dendritic cells. In some embodiments, peptide antigens can displace antigens previously bound to MHC molecules on the surface of dendritic cells. Thus, the antigen may be processed by the dendritic cells and presented or may be loaded onto MHC molecules on the surface of dendritic cells without processing. Those peptide(s) that can be presented by the dendritic cell may appear on the surface in the context of MHC molecules for presentation to T cells. This can be demonstrated functionally (e.g., by measuring T cell responses to the cell) or by detecting antigen-MHC complexes using methods known in the art. This can also be demonstrated functionally by assessing the generation of one or more tolerogenic immune response by the antigen-specific itDCs (e.g., ability to activate antigen-specific T or B cells). Such methods include assessing the level and/or function of therapeutic protein in a subject. Other methods are described elsewhere herein.

In some embodiments, cells are contacted with an antigen comprising more than one protein or more than one polypeptide or more than one peptide and the antigen is not purified to remove irrelevant or unwanted proteins, polypeptides, or peptides and the cells present those antigens which are processed and displayed. In some embodiments, the antigen used to contact dendritic cells comprises or consists of a single short peptide or polypeptide or mixture of peptides or polypeptides that are substantially pure, e.g., isolated from contaminating peptides or polypeptides. Likewise, the antigen can be a single polypeptide or peptide that is substantially pure and isolated from contaminating polypeptides or peptides. Such short peptides and polypeptides can be obtained by suitable methods known in the art. For example, short peptides or polypeptides can be recombinantly expressed, purified from a complex protein antigen, or produced synthetically.

Alternatively, the antigen used to contact cells comprises or consists of a mixture of more than one short peptide or polypeptide, e.g., a mixture of two, three, four, five, six, seven, eight, nine, ten, twenty, thirty, forty, fifty, one hundred or more short peptides or polypeptides. The antigen used to contact cells can also comprise or consist of a more complex mixture of polypeptides. Use of a mixture of short peptides or polypeptides allows for the preparation of an induced dendritic cell population that is capable of, for example, modulating an antigen-specific T-cell mediated immune response to a number of distinct peptides or polypeptides. This is desirable when, for example, the immune response to be inhibited is an immune response against a complex antigen or particular cell types. In some embodiments, the antigen comprises a cell extract or cell lysate. In some embodiments, the antigen comprises a tissue extract or tissue lysate.

Other methods of loading antigen onto dendritic cells will be apparent to one of ordinary skill in the art (See, e.g., Dieckman et al. Int. Immunol. (May 2005) 17(5): 621-635).

In some embodiments, the antigen is associated with allergic responses. In such embodiments, the antigen with which the dendritic cells are contacted with can comprise one or more allergens (e.g., one or more polypeptides or peptides derived therefrom). In some embodiments, the antigen is a complex antigen, such as: a food protein (e.g., one or more proteins peptides or polypeptides derived from food, such as eggs, milk, wheat, soy, nuts, seeds, fish, shellfish, or gluten), pollen, mold, dust mites, or particular cell types or cells modified by exposure to a drug or chemical.

In some embodiments, the antigen comprises animal matter, such as one or more of animal dander, hair, urine or excrement. In some embodiments, the antigen comprises insect matter.

In some embodiments, the antigen comprises or consists of one or more peptides or polypeptides derived from food. In still some embodiments, the antigen comprises one or more peptides or polypeptides derived pollen. In some embodiments, the antigen comprises one or more peptides or polypeptides derived dust mites. In some embodiments, the antigen comprises one or more peptides or polypeptides derived gluten. In some embodiments, the antigen comprises one or more peptides or polypeptides derived myelin.

In exemplary embodiments, the antigen (or one of the antigens) with which the dendritic cells are contacted in the foregoing methods is an antigen that is targeted by the immune system of a subject with the disease, e.g., targeted by effector T cells, and such targeting contributes to disease progression. Some exemplary antigens of this kind are described herein. Additional antigens of this kind are well known to those of skill in the art, and the invention is not limited in this respect. For example, in some embodiments, the antigen is associated with celiac disease (CD). In such embodiments, the antigen with which the dendritic cells are contacted can be derived from wheat, rye, or barley. In exemplary embodiments, the antigen can comprise gluten or gliadin, or portions or mixtures thereof, for example, amino acids spanning from about amino acid 57 to amino acid 73 of A-gliadin.

In some embodiments, the antigen is associated with type I diabetes. In such embodiments, the antigen with which the dendritic cells are contacted can be one or more peptides or polypeptides derived from islet cells of the pancreas, e.g., can be a cell or tissue lysate or extract; a mixture of proteins or polypeptides or peptides; or one or more purified proteins, polypeptides or peptides.

In some embodiments, the antigen is associated with multiple sclerosis. In such embodiments, the antigen with which the dendritic cells are contacted can be one or more peptides or polypeptides derived from neural cell or tissue. For example, the antigen can be derived from axons, dendrites, neuronal cell bodies, oligodendrocytes, glia cells, microglia or Schwann cells. In particular embodiments, the antigen is myelin, or a component thereof, e.g., myelin basic protein.

In some embodiments, the antigen is associated with primary biliary cirrhosis. In such embodiments, the antigen with which the dendritic cells are contacted can be one or more peptides or polypeptides derived from bile duct cells, e.g., as a cell or tissue lysate or extract.

Other antigens that can be used with the methods of the invention can be envisioned by a person of skill in the art. For example, many autoimmune disorders have been associated with particular proteins, although specific peptide antigens important in such immune responses may not yet be known. Since proteins or mixtures of proteins can be used as antigen in the methods of the instant invention, one of skill in the art could readily determine what antigen or antigen mixture to use for loading dendritic cells to modulate immune responses to that particular antigen.

A wide range of antigen quantities can be used to contacting with the itDCs. For example, in some embodiments, cells are contacted with antigen at concentrations ranging between 1 pg/mL and 10 mg/mL. In exemplary embodiments, cells are contacted with antigen at 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 30 μg/ml, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In some embodiments, cells are contacted with 100 μg/mL of antigen. In some embodiments, cells are contacted with antigen at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein.

In some embodiments, cells can be cocultured with antigen for a time sufficient to allow display of the antigen on the surface of the cells, e.g., 1-72 hours under appropriate conditions (e.g., 37° C. in 5% CO2 atmosphere). For example, in some embodiments, cells are cocultured with antigen for about 1-72 hours, e.g., 1, 2, 3, 4, 5, 6, 7, 8, 9, 10, 12, 20, 24, 30, 35, 40, 45, 48, 50, 55, 60, 70, or 72 hours or such other time period which allows for processing and presentation or loading of antigen onto dendritic cells. Preferably, in some embodiments, the time sufficient is at least 2 hours. In other embodiments, the time sufficient is overnight. In yet other embodiment, the time sufficient is between 2 and 24 or between 2 and 12 hours. Such contacting can take place prior to induction of DCs or after induction and prior to further manipulation.

In some embodiments, the itDCs can be contacted with one or more maturation stimuli prior to administration to a subject. Treatment with a maturation stimulus can enhance the antigen presentation capacity of dendritic cells without blocking their tolerogenicity in the case of induced tolerogenic dendritic cells. Such maturation stimuli can include, but are not limited to, an adjuvant, a TLR agonist, a CD40 agonist, an inflammasome activator, or an inflammatory cytokine, and combinations thereof. Treatment of cells with maturation stimuli can be performed before, during, or following induction and/or contacting with antigen.

In some embodiments, the itDCs and/or therapeutic protein and/or transplantable graft, etc. are administered to a subject by an appropriate route. The administering of the itDCs and/or transplantable graft and/or therapeutic protein, when expressed in a cell and administered as such, may be by parenteral, intraarterial, intranasal or intravenous administration or by injection to lymph nodes or anterior chamber of the eye or by local administration to an organ or tissue of interest. The administering may also be by subcutaneous, intrathecal, intraventricular, intramuscular, intraperitoneal, intracoronary, intrapancreatic, intrahepatic or bronchial injection. Administration can be rapid or can occur over a period of time.

When not administered in cellular form, other agents may be administered by a variety of routes of administration, including but not limited to intraperitoneal, subcutaneous, intramuscular, intradermal, oral, intranasal, transmucosal, intramucosal, intravenous, sublingual, rectal, ophthalmic, pulmonary, transdermal, transcutaneous or by a combination of these routes. Routes of administration also include administration by inhalation or pulmonary aerosol. Techniques for preparing aerosol delivery systems are well known to those of skill in the art (see, for example, Sciarra and Cutie, “Aerosols,” in Remington's Pharmaceutical Sciences, 18th edition, 1990, pp. 1694-1712; incorporated by reference). Other agents can likewise be administered by such routes.

The compositions of the inventions can be administered in effective amounts, such as the effective amounts described elsewhere herein. Doses contain varying amounts of populations of itDCs and/or varying amounts of therapeutic proteins or transplantable grafts according to the invention. The amount of the cells or other agents present in the inventive dosage forms can be varied according to the nature of the cells, antigens, the therapeutic benefit to be accomplished, and other such parameters. In some embodiments, dose ranging studies can be conducted to establish optimal therapeutic amount of the population of cells and/or the other agents to be present in the dosage form. In some embodiments, itDCs and/or the other agents are present in the dosage form in an amount effective to generate a tolerogenic immune response upon administration to a subject. It may be possible to determine amounts of the cells and/or other agents effective to generate a tolerogenic immune response using conventional dose ranging studies and techniques in subjects. Inventive dosage forms may be administered at a variety of frequencies. In a preferred embodiment, at least one administration of the dosage form is sufficient to generate a pharmacologically relevant response. In more preferred embodiments, at least two administrations, at least three administrations, or at least four administrations, of the dosage form are utilized to ensure a pharmacologically relevant response.

The quantity of itDCs to be administered to a subject can be determined by one of ordinary skill in the art. In some embodiments, amounts of cells can range from about 10⁵ to about 10¹⁰ cells per dose. In exemplary embodiments, induced dendritic cells are administered in a quantity of about 10⁵, 10⁶, 10⁷, 10⁸, 10⁹, or 10¹⁰ cells per dose. In other exemplary embodiments, intermediate quantities of cells are employed, e.g., 5×10⁵, 5×10⁶, 5×10⁷, 5×10⁸, 5×10⁹, or 5×10¹⁰ cells. In some embodiments, subjects receive a single dose. In some embodiments, subjects receive multiple doses. Multiple doses may be administered at the same time, or they may be spaced at intervals over a number of days. For example, after receiving a first dose, a subject may receive subsequent doses of itDCs at intervals of 1, 2, 3, 4, 5, 6, 7, 8, 9, 10, 11, 12, 13, 14, 21, 28, 30, 45, 60, or more days. As will be apparent to one of skill in the art, the quantity of cells and the appropriate times for administration may vary from subject to subject depending on factors including the duration and severity of disease, disorder or condition. To determine the appropriate dosage and time for administration, skilled artisans may employ conventional clinical and laboratory means for monitoring the outcome of administration, e.g., on progression of a disorder in the subject or on humoral immune responses, Treg cell, Breg cell, B cell and/or T cell effector number and/or function, etc. Such means include known biochemical and immunological tests for monitoring and assessing, for example, cytokine production, antibody production, inflammation, T-effector cell activity, organ or tissue rejection, allergic response, therapeutic protein level and/or function, etc.

In some embodiments, a maintenance dose is administered to a subject after an initial administration has resulted in a tolerogenic response in the subject, for example to maintain the tolerogenic effect achieved after the initial dose, to prevent an undesired immune reaction in the subject, or to prevent the subject becoming a subject at risk of experiencing an undesired immune response or an undesired level of an immune response. In some embodiments, the maintenance dose is the same dose as the initial dose the subject received. In some embodiments, the maintenance dose is a lower dose than the initial dose. For example, in some embodiments, the maintenance dose is about ¾, about ⅔, about ½, about ⅓, about ¼, about ⅛, about 1/10, about 1/20, about 1/25, about 1/50, about 1/100, about 1/1,000, about 1/10,000, about 1/100,000, or about 1/1,000,000(weight/weight) of the initial dose.

Prophylactic administration of induced dendritic cells can be initiated prior to the onset of disease, disorder or condition or therapeutic administration can be initiated after a disorder, disorder or condition is established.

In some embodiments, administration of itDCs is undertaken e.g., prior to administration of a therapeutic protein or transplantable graft or exposure to an allergen. In exemplary embodiments, induced tolerogenic dendritic cells are administered at one or more times including, but not limited to, 30, 25, 20, 15, 14, 13, 12, 11, 10, 9, 8, 7, 6, 5, 4, 3, 2, 1, or 0 days prior to administration of a therapeutic protein or transplantable graft or exposure to an allergen. In addition or alternatively, itDCs can be administered to an subject concomitantly with or following administration of a therapeutic protein or transplantable graft or exposure to an allergen. In exemplary embodiments, itDCs are administered at one or more times including, but not limited to, 1, 2, 3, 4, 5, 6, 7, 8, 9, 10, 11, 12, 13, 14, 15, 20, 25, 30, etc. days following administration of a therapeutic protein or transplantable graft or exposure to an allergen.

In some embodiments, the use of itDCs will allow for administration of lower doses than that of immunosuppressants of the current standard of care, thereby reducing side effects.

It is to be understood that the cell populations, for example, compositions, and dosage forms of the invention can be made in any suitable manner, and the invention is in no way limited to compositions that can be produced using the methods described herein. Selection of an appropriate method may require attention to the properties of the particular cell populations, compositions, and dosage forms, for example, with regard to their intended use.

For example, in some embodiments, inventive compositions are manufactured under sterile conditions or are generated using sterilized reagents. This can ensure that resulting composition are sterile or non-infectious, thus improving safety when compared to non-sterile compositions. This provides a valuable safety measure, especially when a subject receiving a cell population, composition, or dosage form provided herein has a defective or suppressed immune system, is suffering from infection, and/or is susceptible to infection.

The compositions and methods described herein can be used to induce or enhance a tolerogenic immune response and/or to suppress, modulate, direct or redirect an immune response for the purpose of immune suppression. The compositions and methods described herein can be used in the diagnosis, prophylaxis and/or treatment of diseases, disorders or conditions in which immune suppression or tolerance would confer a treatment benefit. Such diseases, disorders or conditions include inflammatory diseases, autoimmune diseases, allergies, organ or tissue rejection and graft versus host disease. The compositions and methods described herein can also be used in subjects who have undergone or will undergo transplantation. The compositions and methods described herein can also be used in subjects who have received, are receiving or will receive a therapeutic protein against which they have generated or are expected to generate an undesired immune response.

Autoimmune diseases include, but are not limited to, rheumatoid arthritis, multiple sclerosis, immune-mediated or Type I diabetes mellitus, inflammatory bowel disease (e.g., Crohn's disease or ulcerative colitis), systemic lupus erythematosus, psoriasis, scleroderma, autoimmune thyroid disease, alopecia greata, Grave's disease, Guillain-Barré syndrome, celiac disease, Sjögren's syndrome, rheumatic fever, gastritis, autoimmune atrophic gastritis, autoimmune hepatitis, insulitis, oophoritis, orchitis, uveitis, phacogenic uveitis, myasthenia gravis, primary myxoedema, pernicious anemia, autoimmune haemolytic anemia, Addison's disease, scleroderma, Goodpasture's syndrome, nephritis, for example, glomerulonephritis, psoriasis, pemphigus vulgaris, pemphigoid, sympathetic opthalmia, idiopathic thrombocylopenic purpura, idiopathic feucopenia, Wegener's granulomatosis and poly/dermatomyositis.

Some additional exemplary autoimmune diseases, associated autoantigens, and autoantibodies, which are contemplated for use in the invention, are described in Table 1 below:

| Autoantibody Type | Autoantibody | Autoantigen | Autoimmune disease or disorder |
|---|---|---|---|
| Antinuclear | Anti-SSA/Ro | ribonucleoproteins | Systemic lupus erythematosus, neonatal |
| antibodies | autoantibodies | | heart block, primary Sjögren's syndrome |
| | Anti-La/SS-B | ribonucleoproteins | Primary Sjögren's syndrome |
| | autoantibodies | | |
| | Anti-centromere | centromere | CREST syndrome |
| | antibodies | | |
| | Anti-neuronal | Ri[disambiguation | Opsoclonus |
| | nuclear antibody-2 | needed] | |
| | Anti-dsDNA | double-stranded | SLE |
| | | DNA | |
| | Anti-Jo1 | histidine-tRNA | Inflammatory myopathy |
| | | ligase | |
| | Anti-Smith | snRNP core proteins | SLE |
| | Anti- | Type I | Systemic sclerosis (anti-Scl-70 antibodies) |
| | topoisomerase | topoisomerase | |
| | antibodies | | |
| | Anti-histone | histones | SLE and Drug-induced LE[2] |
| | antibodies | | |
| | Anti-p62 | nucleoporin 62 | Primary biliary cirrhosis[3][4][5] |
| | antibodies [3] | | |
| | Anti-sp100 | Sp100 nuclear | |
| | antibodies [4] | antigen | |
| | Anti-glycoprotein- | nucleoporin 210kDa | |
| | 210 antibodies[5] | | |
| Anti- | Anti-tTG | | Coeliac disease |
| transglutaminase | Anti-eTG | | Dermatitis herpetiformis |
| antibodies | | | |
| Anti-ganglioside | | ganglioside GQ1B | Miller-Fisher Syndrome |
| antibodies | | ganglioside GD3 | Acute motor axonal neuropathy (AMAN) |
| | | ganglioside GM1 | Multifocal motor neuropathy with |
| | | | conduction block (MMN) |
| Anti-actin | | actin | Coeliac disease anti-actin antibodies |
| antibodies | | | correlated with the level of intestinal |
| | | | damage [6][7] |
| Liver kidney | | | Autoimmune hepatitis.[8] |
| microsomal type 1 | | | |
| antibody | | | |
| Lupus anticoagulant | Anti-thrombin | thrombin | Systemic lupus erythematosus |
| | antibodies | | |
| Anti-neutrophil | | phospholipid | Antiphospholipid syndrome |
| cytoplasmic | c-ANCA | proteins in | Wegener's granulomatosis |
| antibody | | neutrophil | |
| | | cytoplasm | |
| | p-ANCA | neutrophil | Microscopic polyangiitis, Churg-Strauss |
| | | perinuclear | syndrome, systemic vasculitides (non- |
| | | | specific) |
| Rheumatoid factor | | IgG | Rheumatoid arthritis |
| Anti-smooth muscle | | smooth muscle | Chronic autoimmune hepatitis |
| antibody | | | |
| Anti-mitochondrial | | mitochondria | Primary biliary cirrhosis[9] |
| antibody | | | |
| Anti-SRP | | signal recognition | Polymyositis[10] |
| | | particle | |
| | | exosome complex | Scleromyositis |
| | | nicotinic | Myasthenia gravis |
| | | acetylcholine | |
| | | receptor | |
| | | muscle-specific | Myasthenia gravis |
| | | kinase (MUSK) | |
| Anti-VGCC | | voltage-gated | Lambert-Eaton myasthenic syndrome |
| | | calcium channel | |
| | | (P/Q-type) | |
| | | thyroid peroxidase | Hashimoto's thyroiditis |
| | | (microsomal) | |
| | | TSH receptor | Graves' disease |
| | | Hu | Paraneoplastic cerebellar syndrome |
| | | Yo (cerebellar | Paraneoplastic cerebellar syndrome |
| | | Purkinje Cells) | |
| | | amphiphysin | Stiff person syndrome, paraneoplastic |
| | | | cerebellar syndrome |
| Anti-VGKC | | voltage-gated | Limbic encephalitis, Isaac's Syndrome |
| | | potassium channel | (autoimmune neuromyotonia) |
| | | (VGKC) | |
| | | basal ganglia | Sydenham's chorea, paediatric autoimmune |
| | | neurons | neuropsychiatric disease associated with |
| | | | Streptococcus (PANDAS) |
| | | N-methyl-D- | Encephalitis |
| | | aspartate receptor | |
| | | (NMDA) | |
| | | glutamic acid | Diabetes mellitus type 1, stiff person |
| | | decarboxylase | syndrome |
| | | (GAD) | |
| | | aquaporin-4 | Neuromyelitis optica (Devic's syndrome) |

Inflammatory diseases include, but are not limited to, Alzheimer's, Ankylosing spondylitis, arthritis, asthma, atherosclerosis, Behcet's disease, chronic inflammatory demyelinating polyradiculoneuropathy, Crohn's disease, colitis, cystic fibrosis, dermatitis, diverticulitis, hepatitis, irritable bowel syndrome (IBS), lupus erythematous, muscular dystrophy, nephritis, Parkinson's, shingles and ulcerative colitis. Inflammatory diseases also include, for example, cardiovascular disease, chronic obstructive pulmonary disease (COPD), bronchiectasis, chronic cholecystitis, tuberculosis, Hashimoto's thyroiditis, sepsis, sarcoidosis, silicosis and other pneumoconioses, and an implanted foreign body in a wound, but are not so limited. As used herein, the term “sepsis” refers to a well-recognized clinical syndrome associated with a host's systemic inflammatory response to microbial invasion. The term “sepsis” as used herein refers to a condition that is typically signaled by fever or hypothermia, tachycardia, and tachypnea, and in severe instances can progress to hypotension, organ dysfunction, and even death.

In some embodiments, the inflammatory disease is non-autoimmune inflammatory bowel disease, post-surgical adhesions, coronary artery disease, hepatic fibrosis, acute respiratory distress syndrome, acute inflammatory pancreatitis, endoscopic retrograde cholangiopancreatography-induced pancreatitis, burns, atherogenesis of coronary, cerebral and peripheral arteries, appendicitis, cholecystitis, diverticulitis, visceral fibrotic disorders, wound healing, skin scarring disorders (keloids, hidradenitis suppurativa), granulomatous disorders (sarcoidosis, primary biliary cirrhosis), asthma, pyoderma gandrenosum, Sweet's syndrome, Behcet's disease, primary sclerosing cholangitis or an abscess. In some preferred embodiment the inflammatory disease is inflammatory bowel disease (e.g., Crohn's disease or ulcerative colitis).

In other embodiments, the inflammatory disease is an autoimmune disease. The autoimmune disease in some embodiments is rheumatoid arthritis, rheumatic fever, ulcerative colitis, Crohn's disease, autoimmune inflammatory bowel disease, insulin-dependent diabetes mellitus, diabetes mellitus, juvenile diabetes, spontaneous autoimmune diabetes, gastritis, autoimmune atrophic gastritis, autoimmune hepatitis, thyroiditis, Hashimoto's thyroiditis, insulitis, oophoritis, orchitis, uveitis, phacogenic uveitis, multiple sclerosis, myasthenia gravis, primary myxoedema, thyrotoxicosis, pernicious anemia, autoimmune haemolytic anemia, Addison's disease, Anklosing spondylitis, sarcoidosis, scleroderma, Goodpasture's syndrome, Guillain-Barré syndrome, Graves' disease, glomerulonephritis, psoriasis, pemphigus vulgaris, pemphigoid, excema, bulous pemiphigous, sympathetic opthalmia, idiopathic thrombocylopenic purpura, idiopathic feucopenia, Sjögren's syndrome, systemic sclerosis, Wegener's granulomatosis, poly/dermatomyositis, primary biliary cirrhosis, primary sclerosing cholangitis, lupus or systemic lupus erythematosus.

Graft versus host disease (GVHD) is a complication that can occur after a pluripotent cell (e.g., stem cell) or bone marrow transplant in which the newly transplanted material results in an attack on the transplant recipient's body. In some instances, GVHD takes place after a blood transfusion. Graft-versus-host-disease can be divided into acute and chronic forms. The acute or fulminant form of the disease (aGVHD) is normally observed within the first 100 days post-transplant, and is a major challenge to transplants owing to associated morbidity and mortality. The chronic form of graft-versus-host-disease (cGVHD) normally occurs after 100 days. The appearance of moderate to severe cases of cGVHD adversely influences long-term survival.

### EXAMPLES

### Example 1

### Isolation of a Starting Population of Cells (Prophetic)

Starting populations are obtained from the bone marrow, the peripheral blood, or the spleen of a donor subject. In case of solid tissue being harvested or obtained from a subject, the tissue is digested or mechanically disrupted in order to obtain a cell suspension, for example, a single-cell suspension. In case of bone marrow or peripheral blood, the cells are separated from the non-cellular components and undesired cells, e.g., erythrocytes, B-lymphocytes and granulocytes are depleted. Bone marrow and peripheral blood cell populations are depleted of erythrocytes by hypotonic lysis. Erythroid precursors, B lymphocytes, T-lymphocytes, and granulocytes are removed by immunomagnetic bead depletion.

The obtained cell populations are enriched for dendritic cells and/or dendritic cell precursors by cell sorting for CD11c. For cell sorting, FACS or MACS are used in combination with a CD11c-antibody or CD11c immunomagnetic beads, respectively. Enriched populations of dendritic cells or dendritic cell precursors are more than 90% pure. Dendritic cell populations and dendritic precursor cell populations are cultured in a suitable culture medium until further processing, e.g., in RPMI-1640 with 10% fetal calf serum, 1-glutamine, non-essential amino acids, sodium pyruvate, penicillin-streptomycin, HEPES, 2-mercaptoethanol, 1000 U/mL recombinant human granulocyte-macrophage colony-stimulating factor, and 1000 U/mL recombinant human IL-4 at 37° C.

### Example 2

### Isolation of Subsets of Dendritic Cells (Prophetic)

Subsets of DCs are isolated from peripheral blood. For each cell isolation, about 10⁶ peripheral blood mononuclear cells (PBMCs) are used as starting material. The cellular fraction of the peripheral blood is separated from the blood plasma and serum by centrifugation and washing with PBS containing BSA. Erythrocytes are lysed by incubation of blood cells with erythrocyte lysis buffer (8.26 g/l ammonium chloride (NH4Cl); 1 g/l potassium bicarbonate (KHCO3); 0.037/1 g EDTA in H₂O). After erythrocyte lysis, intact cells are washed again. Alternatively, PBMCs are isolated by Ficoll-Hypaque density gradient centrifugation from peripheral blood. The starting populations of PBMCs contain dendritic cells and numerous unwanted cell types. The unwanted cell types are depleted by negative selection. The desired DCs of a specific DC subset, e.g., plasmacytoid DCs or XCR1+ DCs, can also be isolated based on positive selection. In embodiments, where “untouched” DCs are desired, the desired DCs are not labeled with any binding agent, but enriched for by depletion of undesired cells only. Depletion of undesired cells is achieved by labeling undesired cells with a binding agent specifically binding the undesired cells, but not the desired cells, for example, an antibody, and conjugating the undesired cells to a detectable or selectable moiety, for example, a fluorescent dye or a magnetic particle. The undesired cells so labeled can then be physically separated from the unlabeled cells of interest, for example, by fluorescent or magnetic cell sorting, e.g., by performing a standard FACS or MACS procedure, respectively.

Primary DCs (BDCA1+ DCs, XCR1(BDCA3)+DCs, and plasmacytoid DCs) are isolated from PBMCs depleted of CD3+ and CD14+ cells using CD3+ and CD14+ MACS microbeads and a standard MACS procedure (Miltenyi Biotec). The DCs so isolated are labeled and further subdivided into one or more of the following subsets by positive and/or negative selection for one or more of the markers they express: BDCA1+ DCs are negative for CD14, CD16, and CD19 and positive for HLA-DR and BDCA1; XCR1+ DCs are positive for HLA-DR and XCR1(BDCA3); and plasmacytoid DCs are positive for HLA-DR and CD123. The following antibodies are used: HLA-DR-PerCp (G46-6), CD14-PE-Cy7 (M5E2), CD16-APC-H7 (3G8; all from BD), CD19-ECD (J3-119; Beckman Coulter), BDCA1-Pacific Blue (L161; BioLegend), BDCA3-APC (AD5-14H12), and CD123-PE (AC145; both from Miltenyi Biotec).

Alternatively, CD4⁺, CD45RA⁺, CD123⁺, ILT3⁺, ILT1⁻, CD11c^{low}, lineage⁻ plasmacytoid dendritic cells are isolated from PBMCs by negative selection. Briefly, peripheral blood cells are incubated with binding agents, for example, antibodies, that specifically bind unwanted cells, for example, T cells, B cells, NK cells, myeloid dendritic cells, monocytes, granulocytes, and erythroid cells. Unwanted non-pDCs are indirectly magnetically labeled by using a cocktail of biotin-conjugated antibodies and anti-biotin-coated magnetic beads. Using a magnetic column separator in a magnetism-activated cell sorting (MACS) procedure, the unwanted labeled cells are retained in the column, while the unlabeled pDCs are collected in the flow-through. The isolated pDCs are CD303 (BDCA-2)⁺, CD304 (BDCA-4/Neuropilin-1)⁺, CD123⁺, CD4⁺, CD45RA⁺, CD141 (BDCA-3)^{dim} and CD1c (BDCA-1)⁻, CD2⁻, lack expression of lineage markers (CD3, CD14, CD16, CD19, CD20, CD56), and express neither myeloid markers such as CD13 and CD33, nor Fc receptors such as CD32, CD64, or FcεRI. Any of the listed markers are used in negative or positive selection for pDCs in some embodiments. In some embodiments, pDCs are isolated by negative or positive selection for any of the listed markers using MACS or FACS technology. In some embodiments, a commercial pDC isolation kit is used (e.g., Plasmacytoid dendritic cell isolation kit, Cat#130-092-207, Miltenyi Biotech, Bergisch Gladbach, Germany) according to the manufacturers recommendations.

In some embodiments, XCR1-expressing DCs are isolated from PBMCs prepared by standard Biocoll (Biochrom) density gradient centrifugation of peripheral blood as follows: Whole blood is subjected to erythrocyte lysis with ACK buffer (155 mM NH4C1, 10 mM KHCO3, 0.1 mM EDTA) followed by magnetic or fluorescent cell sorting (CD45+, CD15+, CD3−, CD14−, CD19−, CD141−) to a purity of >99%, as assessed, e.g., by FACS. T cells (CD3+, CD19−, CD141−), B cells (CD19+, HLA-DR+, CD3−, CD141−), NK cells (CD16+, CD56+, CD3−, CD141−, HLA-DR−), and monocytes (CD14+, CD3−, CD19−, CD141−) are depleted from the initial PBMCs to a purity of >99%. CD1c+DCs (CD1c+, CD11c+, HLA-DR+, CD16−, CD141−, lin−), CD16+ DCs (CD16+, CD11c+, HLA-DR+, CD1c−, CD141−, lin−), CD141+ DCs (CD141+, CD11c+, HLA-DR+, CD1c+, CD16−, lin−), and pDCs (CD304+, HLA-DR+, CD11c-, CD141−, lin−) are sorted by performing a MACS procedure from PBMCs after magnetic enrichment with the respective dendritic cell isolation kit from Miltenyi Biotec.

In some embodiments, pDCs are isolated from human PBMCs using positive and negative selection. First, B cells and monocytes are magnetically labeled and depleted using a cocktail of CD19 and CD14 antibodies conjugated to magnetic particles. Subsequently, the pre-enriched dendritic cells in the non-magnetic flow-through fraction are magnetically labeled and enriched using a cocktail of antibodies against the dendritic cell markers CD304 (BDCA-4/Neuropilin-1), CD141 (BDCA-3), and CD1c (BDCA-1). The highly pure enriched cell fraction comprises plasmacytoid dendritic cells, CD1c (BDCA-1)+ type-1 myeloid dendritic cells (MDC1s), and CD1c (BDCA-1)− CD141 (BDCA-3)^{bright} type-2 myeloid dendritic cells (MDC2s). B cells and monocytes are depleted in advance because a subpopulation of B cells expresses CD1c (BDCA-1), and monocytes express CD141 (BDCA-3) at low levels. In some embodiments, the Blood dendritic cell isolation kit II from Miltenyi Biotec (Cat #130-091-379) is used for the isolation of pDCs according to the manufacturer's recommendations.

### Example 3

### Induction of itDCs (Prophetic)

Starting populations of dendritic cells or dendritic precursor cells are contacted with a tolerogenic stimulus, here, with the mTOR inhibitor rapamycin and TGFβ at 10 ng/ml each for 1 h. An appropriate volume of a concentrated stock solution (e.g., 1000×) of each agent is added to the supernatant of the culture of the starting population to achieve the desired end concentration of the agent in the tissue culture medium. After the contacting time period has elapsed, cells are washed three times with PBS and transferred to culture medium not containing the tolerogenic stimulus. Respirostatic characteristics of the tolerogenic induction is monitored by assessing O₂ consumption of the cell populations.

For DC precursors, after seven days in culture, tolerogenic characteristics of the DCs is assessed by contacting a population of naïve T cells with some of the DCs generated and measuring induction of FoxP3 in the naïve T cells, wherein cell populations containing cells that induce FoxP3 contain itDCs.

### Example 4

### Antigen-Loading of itDCs (Prophetic)

Cultures of itDCs are contacted with a transplant antigen of interest by contacting the itDCs with a crude lysate of differentiated pluripotent transplantable cells expressing the antigen. The itDCs are contacted for with the crude lysate for 24 h at 37° C., and subsequently washed three times in PBS. Antigen-loaded itDCs are then cultured, or used according to methods described herein.

### Example 5

### Evaluating Tolerogenic Immune Response by T-Cell Phenotypic Analysis (Prophetic)

A composition of the invention is injected subcutaneously into female Lewis rats. A control group of rats receives 0.1-0.2 ml of PBS. Nine to ten days after the injection, spleen and lymph nodes are harvested from the rats and single cell suspensions obtained by macerating tissues through a 40 μm nylon cell strainer. Samples are stained in PBS (1% FCS) with the appropriate dilution of relevant monoclonal antibodies. Propidium iodide staining cells are excluded from analysis. Samples are acquired on an LSR2 flow cytometer (BD Biosciences, USA) and analyzed using FACS Diva software. The expression of markers CD25^{high}, CD27^{high}, CD86^{high}, CD1d^{high}, IL-10^{high}, TGF-β^{high}, CD4 and FoxP3 is analyzed on the cells. The presence of CD8+ CD25 highFoxP3+ cells suggests an induction of CD8+ Treg cells.

### Example 6

### Evaluating Tolerogenic Immune Response to Antigen In Vivo (Prophetic)

Balb/c mice are immunized with an autoantigen in incomplete Freund's adjuvant to induce antigen-specific T-cell proliferation (e.g., CD8+ T-cell), the level of which is assessed. Subsequently, a composition of the invention is administered in a dose-dependent manner. The same mice are then again exposed to the autoantigen, and the level of T-cell proliferation is again assessed. Changes in the T-cell population are then monitored with a reduction in T-cell proliferation upon subsequent challenge with the antigen indicating a tolerogenic immune response.

### Example 7

### Administration to a Subject to Suppress an Undesired Immune Response (Prophetic)

Antigen-specific itDCs are formulated into a dosage form suitable for administration (e.g., an injectable cell suspension) and an effective amount of the dosage form is administered to a subject having a disease associated with an undesired immune response, for example, type I Diabetes.

### Example 8

### Administration to a Subject to Suppress an Undesired Immune Response Against an Antigen (Prophetic)

Antigen-specific itDCs are formulated into a dosage form suitable for human administration. The composition is administered to the subject as an injectable cell suspension. Epoietin alfa-specific itDCs are generated according to methods described herein. Briefly, itDCs are generated by contacting itDCs with epoietin alfa or portion thereof. Epoietin alfa-specific itDCs are then formulated into an injectable cell suspension of about 10⁶ cells/ml in sterile, injectable saline. An effective amount of this injectable suspension, about 1 ml, is administered subcutaneously to a subject receiving epoietin alfa as part of a therapeutic schedule, and exhibiting an undesired immune response, such as an excessive epoietin alfa-specific antibody production or CD4+ T cell proliferation and/or activity. A decrease in these undesired immune responses against the therapeutic protein is expected in the subject after about one to four weeks after administration of the epoietin alfa-specific itDCs. This decrease is expected to result in an ameliaration or complete regression of epoietin alfa-specific antibody production or CD4+ T cell proliferation and/or activity. Methods of assessing the level of epoietin alfa-specific antibody production or CD4+ T cell proliferation and/or activity are provided elsewhere herein or are otherwise known to those of ordinary skill in the art.

### Example 9

### Generation of Antigen-Specific itDCs from a Specific Subset of DCs and Administration to a Subject to Suppress an Undesired Immune Response (Prophetic)

For DC subset isolation, about 10⁶-10¹⁰ peripheral blood mononuclear cells (PBMCs) obtained from a subject are used as starting material. The cellular fraction of the peripheral blood is separated from the blood plasma and serum by centrifugation and washing with PBS containing BSA. Erythrocytes are lysed by incubation of blood cells with erythrocyte lysis buffer (8.26 g/l ammonium chloride (NH4Cl); 1 g/l potassium bicarbonate (KHCO3); 0.037/1 g EDTA in H₂O). After erythrocyte lysis, intact cells are washed again.

Primary DCs (BDCA1+ DCs, XCR1(BDCA3)+DCs, and plasmacytoid DCs) are isolated from PBMCs depleted of CD3+ and CD14+ cells using CD3+ and CD14+ MACS microbeads and a standard MACS procedure (Miltenyi Biotec). From the DCs so isolated, a subset of BDCA1+ DCs is isolated. These DCs are negative for CD14, CD16, and CD19 and positive for HLA-DR and BDCA1.

The BDCA1+ DC are exposed to a tolerogenic stimulus and loaded with epoietin alpha to generate antigen-specific BDCA1+ itDCs according to methods described herein. BDCA1+ antigen-specific itDCs are then formulated into a an injectable cell suspension of about 10⁶ cells/ml in sterile, injectable saline. An effective amount of this injectable suspension, about 1 ml, is administered to a subject subcutaneously having anemia. A decrease in the level of an undesired immune reaction to epoietin alpha, or a complete suppression of the undesired immune response is expected in the subject after about one to four weeks after administration of the itDCs. For one year after administration of the initial dose of itDCs, the subject receives a bi-monthly maintenance dose of about 10⁷ BDCA1+ antigen-specific itDCs (a total of 6 maintenance doses). At the end of this treatment schedule, the subject is expected to show no or only a tolerable immune reaction to the epoietin alpha.

### Example 10

### Pluripotent Cell-Derived, Lineage Specific Cellular Antigen Source (Prophetic)

In order to induce integral tolerance to a multitude of antigens expressed by differentiated cells, plutipotent stem cells can be induced to differentiate in vitro in order to obtain significant numbers of these lineage specific cells. As an example mesenchymal stem cells (MSC) can be isolated from bone marrow or the blood and induced to differentiate into beta cells similar to those found in the pancreatic islet. Stem cells (Nestin+ or not) are treated with a series of compounds and steps including trichostatin A, 5-aza 2′ deoxycytidine nicotinamide and all-trans retinoic acid to induce this differentiation (J. Endocrinol. 2011 May; 209(2):193-201. Epub 2011 Feb. 17; World J Gastroenterol 2004; 10(20):3016-3020). At the end of the procedure genetic and phenotypic marquers are those of beta cell pancreatic, insulin producing cells. These can serve as a source of antigens by using extracts to load in induced tolerogenic dendritic cells (itDC). Briefly, in vitro differentiated beta cells can be lysed by consecutive freeze-thaw cycles and the water-soluble fraction (mainly protein) isolated by slow centrifugation (preserving the supernatant) and filtration over 40 μm cellular sieves. After dosing the content in protein, DC are loaded by adding 10 μg/ml in protein content of the lysis extract during incubation to differentiate normal DC into itDC. These are then administered to induce broad tolerance to all beta cell antigens in individuals suffering from autoimmune diabetes. Diabetes is evaluated by closely following blood sugar levels in these individuals until hyperglycemia is controlled and the autoimmune response eliminated.

### Example 11

### Induced Tolerogenic itDCs Suppress Undesired Immune Responses to Antigen

In vitro Treatment of DCs to Yield Induced Tolerigenic DCs (itDCs)

DCs were incubated for 2 hours under tissue culture conditions (37° C., 5% CO₂) in Complete Media (CM, RPMI1640+10% Fetal Bovine Serum+Penicillin Streptomycin+L-Glutamate) with Rapamycin, (100 nM) TGFβ (2 ong/ml) and Ova peptide (323-339) (1 uM). Cells were then washed 3 times in CM and counted. Cells were placed at 1-10×10⁶/200 ul in PBS and injected i.v. into experimental recipients.

### Immunization and Treatment

Group #1 of animals remained unimmunized as a control. All other groups were immunized using active immunization with OVA protein and CpG subcutaneously in the subscapular region (25 μg OVA+20 μg CpG/animal). Group #2 were immunized but not treated to help appreciate the strength of the immune response induced. Groups #3-10 were treated (200 μl DC i.v.) with different itDC products. The challenge route of administration was 20 μl/limb of OVA (10 μg) or PBS. Five animals per group.

Treatments were carried out concomitantly with immunizations starting on day 0 as follows for the denoted groups. DCs used to treat groups 2-10 were incubated with 10 ug OVA+/−100 ng/ml Rapa and 20 ng/ml TGFβ per animal.

1) Phosphate buffered saline (PBS), intravenously (i.v.),

2) Phosphate buffered saline (PBS), i.v.,

3) Dendritic cells (DCs) incubated with OVA in vitro, i.v.,

4) DCs incubated with OVA, Rapamycin (Rapa) and Tumor Growth Factor beta (TGFβ) in vitro, i.v.,

5) DCs incubated with nanoparticles containing OVA (NPOVA) in vitro, i.v.,

6) DCs incubated with NPOVA, Rapa and TGFβ in vitro, i.v.,

7) CD8 alpha positive (CD8a) DCs incubated with OVA in vitro, i.v.,

8) CD8a DCs incubated with OVA, Rapamycin (Rapa) and Tumor Growth Factor beta (TGFβ) in vitro, i.v.,

9) CD103 positive (CD103) DCs incubated with OVA in-vitro, i.v.,

10) CD103 DCs incubated with OVA, Rapamycin (Rapa) and Tumor Growth Factor beta (TGFβ) in vitro, i.v.

For each treatment day syngeneic donor mice were inoculated 10 days earlier with Fms-like tyrosine kinase 3 (FLT-3) ligand expressing melanoma cells s.s. (performed on days−10, 4, 18 in donor C57BL/6 age-matched mice). Flt3 ligand is a growth factor for DCs and allows for greater total number of DCs to be present in the spleen. This increased the number of DCs more than 10-fold and allowed for more cells to be available for in vitro treatment and in vivo administration.

### Cell Harvesting

On day 39 spleens from syngeneic donor mice were harvested. The spleen cells were mashed into a single-cell suspension and split before being labeled with either 0.5 uM or 5 uM carboxyfluorescein succinimidyl esters (CFSE), an intracellular dye that tracks cells in vivo. The population labeled with 0.5 uM CFSE was then further incubated with SIINFEKL (SEQ ID NO:944) peptide, a Major Histocompatibility Complex (MHC) class I restricted peptide from OVA. These two differentially labeled cell populations were filtered and admixed at a 1:1 ratio before being injected i.v. into every mouse within the experiment.

On day 40 all mice were sacked and spleens were harvested. They were stored in PBS before being mashed into a single cell suspension using a syringe plunger and 70 uM sieve. An RBC lyse was then performed using ammonium chloride solution and after washing a portion of the remaining splenocytes was analyzed by flow cytometry.

### Cell Sorting

On treatment days the spleens from the FLT-3 melanoma inoculated animals were harvested and digested via liberase. The resulting slurry was filtered by 70 uM nylon mesh and a series of magnetic activating cell sorting (MACS) separations was performed. First the cells were incubated with magnetic bead conjugated antibodies (Abs) specific for CD45R, DX5 and CD3. These cells were then run through a Miltenyi Biotec Automacs PRO automatic cell separator. The unlabeled cell fraction was then split into 3 groups. The first was incubated with bead conjugated Abs specific for CD11c the second was incubated with bead conjugated Abs specific for CD8a and the third was first incubated with biotin conjugated Abs specific for CD103 and then Abs conjugated to both streptavidin and beads. These cell separations were again performed on the AutoMacs PRO to yield enriched populations of CD11c+, CD8a+ and CD103+ DCs.

### Measurement of Specific Cell Killing

During manual processing of spleens a red blood cell lysis was performed. 3 mls of red blood cell lysis buffer (RBC lyse) was added to a 50 ml polypropylene centrifuge tube then a 70 um sieve was seated on top of the uncapped tube. 1 ml of RBC lyse was pipetted over the sieve and a 5 minute timer was started just before a spleen was placed on it and mashed through it using the plunger of a 3 ml syringe. Once the spleen was completely pulverized and no trace of redness was left in the remaining tissue 1 ml of RBC lyse was pipetted over the sieve to wash out the remaining cells. After 5 minutes had elapsed 5 mls of complete media (RPMI 1640+10% bovine serum v/v+L-glutamate+Penicillin Streptomycin, CM) was added to the tube. Tubes were then moved onto ice until they were spun and the cells pelleted and resuspended in PBS containing 7AAD, a marker of dead cells. Flow cytometry was then performed to determine the proportions of CFSElow versus CFSEhi cells in the experimental animals.

### Measurement of IgG

The level of IgG antibodies were measured. Blocker Casein in PBS (Thermo Fisher, Catalog #37528) was used as diluent. 0.05% Tween-20 in PBS was used as wash buffer, prepared by adding 10 ml of Tween-20 ((Sigma, Catalog #P9416-100 mL) to 2 liters of a 10×PBS stock (PBS: OmniPur® 10×PBS Liquid Concentrate, 4L, EMD Chemicals, Catalog #6505) and 18 Liters of deionized water.

OVA protein at a stock concentration of 5 mg/ml was used as a coating material. A 1:1000 dilution to 5 μg/ml was used as a working concentration. Each well of the assay plates was coated with 100 μl diluted OVA per well, plates were sealed with sealing film (VWR catalog #60941-120), and incubated overnight at 4° C. Costar9017 96-well Flat bottom plates were used as assay plates, Costar9017.

Low-binding polypropylene 96-well plate or tubes were used as set-up plates, in which samples were prepared before being transferred to the assay plate. The setup plates did not contain any antigen and, therefore, serum antibodies did not bind to the plate during the setup of the samples. Setup plates were used for sample preparation to minimize binding that might occur during preparation or pipetting of samples if an antigen-coated plate was used to prepare the samples. Before preparing samples in the setup plate, wells were covered with diluent to block any non-specific binding and the plate was sealed and incubated at 4° C. overnight.

Assay plates were washed three times with wash buffer, and wash buffer was completely aspirated out of the wells after the last wash. After washing, 300 μl diluent were added to each well of assay plate(s) to block non-specific binding and plates were incubated at least 2 hours at room temperature. Serum samples were prepared in the setup plate at appropriate starting dilutions. Starting dilutions were sometimes also prepared in 1.5 ml tubes using diluent. Appropriate starting dilutions were determined based on previous data, where available. Where no previous data was available, the lowest starting dilution was 1:40. Once diluted, 200 μl of the starting dilution of the serum sample was transferred from to the appropriate well of the setup plate.

An exemplary setup plate layout is described as follows: Columns 2 and 11 contained anti-Ovabumin monoclonal IgG2b isotype (AbCam, ab17291) standard, diluted to 1 μg/mL (1:4000 dilution). Columns 3-10 contained serum samples (at appropriate dilutions). Columns 1 and 12 were not used for samples or standards to avoid any bias of measurements due to edge effect. Instead, columns 1 and 12 contained 200 μl diluent. Normal mouse serum diluted 1:40 was used as a negative control. Anti-mouse IgG2a diluted 1:500 from 0.5 mg/mL stock (BD Bioscience) was used as an isotype control.

Once all samples were prepared in the setup plate, the plate was sealed and stored at 4° C. until blocking of the assay plates was complete. Assay plates were washed three times with wash buffer, and wash buffer was completely aspirated after the last wash. After washing, 100 μL of diluent was added to all wells in rows B-H of the assay plates. A 12-channel pipet was used to transfer samples from the setup plate to the assay plate. Samples were mixed prior to transfer by pipetting 150 μl of diluted serum up and down 3 times. After mixing, 1500 of each sample was transferred from the setup plate and added to row A of the respective assay plate.

Once the starting dilutions of each sample were transferred from the setup plate to row A of the assay plate, serial dilutions were pipetted on the assay plate as follows: 50 μl of each serum sample was removed from row A using 12-channel pipet and mixed with the 100 μl of diluent previously added to each well of row B. This step was repeated down the entire plate. After pipetting the dilution of the final row, 50 μl of fluid was removed from the wells in the final row and discarded, resulting in a final volume of 100 μl in every well of the assay plate. Once sample dilutions were prepared in the assay plates, the plates were incubated at room temperature for at least 2 hours.

After the incubation, plates were washed three times with wash buffer. Detection antibody (Goat anti-mouse anti-IgG, HRP conjugated, AbCam ab98717) was diluted 1:1500 (0.33 μg/mL) in diluent and 100 μl of the diluted antibody was added to each well. Plates were incubated for 1 hour at room temperature and then washed three times with wash buffer, with each washing step including a soak time of at least 30 seconds.

After washing, detection substrate was added to the wells. Equal parts of substrate A and substrate B (BD Biosciences TMB Substrate Reagent Set, catalog #555214) were combined immediately before addition to the assay plates, and 100 μl of the mixed substrate solution were added to each well and incubated for 10 minutes in the dark. The reaction was stopped by adding 50 μl of stop solution (2N H2SO4) to each well after the 10 minute period. The optical density (OD) of the wells was assessed immediately after adding the stop solution on a plate reader at 450 nm with subtraction at 570 nm. Data analysis was performed using Molecular Device's software SoftMax Pro v5.4. In some cases, a four-parameter logistic curve-fit graph was prepared with the dilution on the x-axis (log scale) and the OD value on the y-axis (linear scale), and the half maximum value (EC50) for each sample was determined. The plate template at the top of the layout was adjusted to reflect the dilution of each sample (1 per column).

### Results

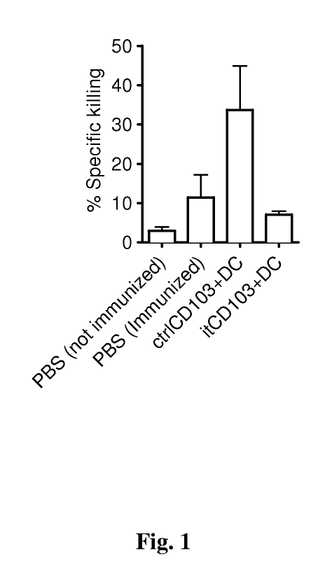

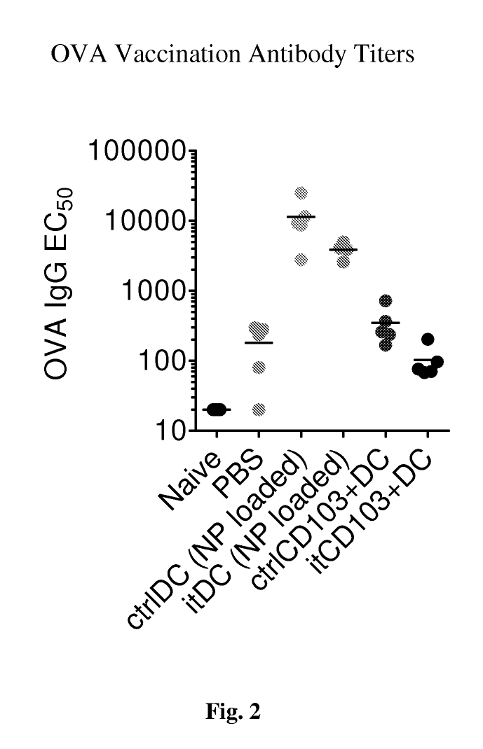

FIG. 1 demonstrates that antigen-specific itDCs, in particular the widely distributed circulating CD103+ subset, effectively reduced the percentage of specific killing of cells expressing antigen. FIG. 2 demonstrates that antigen-specific itDCs of the same subset effectively reduced the production of antigen-specific antibodies.

1. A method comprising:
isolating cells of a dendritic cell subset that possess a desired physiological characteristic, and
generating induced tolerogenic dendritic cells (itDCs) from the cells of the dendritic cell subset.

2. The method of claim 1, wherein the cells of the dendritic cell subset are XCR1+ dendritic cells.

3. The method of claim 1, wherein the cells of the dendritic cell subset are plasmacytoid dendritic cells.

4. The method of claim 1, wherein the cells of the dendritic cell subset are CD103+ dendritic cells.

5. The method of claim 1, wherein the cells of the dendritic cell subset are not enriched for XCR1+ dendritic cells and/or plasmacytoid and/or CD103+ dendritic cells.

6.-7. (canceled)

8. The method of claim 1, wherein the generating comprises contacting the cells of the dendritic cell subset ex vivo with at least one agent that promotes respirostatic tolerance.

9. The method of claim 1, wherein the generating comprises contacting the cells of the dendritic cell subset ex vivo for less than 10 h with a purinergic receptor antagonist, an mTOR inhibitor, a statin or an agent that disrupts mitochondrial electron transport.

10. The method of claim 1, wherein the generating comprises contacting the cells of the dendritic cell subset ex vivo with at least one agent that causes the cells to increase expression of costimulatory molecules while retaining a tolerogenic phenotype upon stimulation with at least one TLR agonist.

11. The method of claim 1, wherein the generating comprises contacting the cells of the dendritic cell subset ex vivo with at least one agent that causes the cells to have the characteristic of i) inducing Foxp3 expression in naïve T cells ex vivo, ii) deleting effector T cells ex vivo and/or iii) converting FoxP3− effector T cells to FoxP3+ effector T cells ex vivo.

12. The method of claim 1, wherein the generating comprises combining the cells of the dendritic cell subset with an antigen.

13. The method of claim 12, wherein the antigen comprises one or more epitopes of a therapeutic protein, a transplantable graft, an autoantigen or an allergen, or is associated with an inflammatory disease, an autoimmune disease, an allergy, organ or tissue rejection or graft versus host disease.

14.-20. (canceled)

21. A method comprising:
administering to a subject dendritic cell subset-enriched itDCs in an amount effective to reduce the generation of an undesired immune response or to generate a desired immune response in the subject.

22. A method comprising:
reducing the generation of an undesired immune response or generating a desired immune response in a subject by administering dendritic cell subset-enriched itDCs to the subject.

23. A method comprising:
administering to a subject dendritic cell subset-enriched itDCs according to a protocol that was previously shown to reduce the generation of an undesired immune response or to generate a desired immune response in one or more test subjects.

24.-37. (canceled)

38. A method comprising administering the dendritic cell subset-enriched itDCs produced by the method of claim 1.

39. A composition comprising dendritic cell subset-enriched itDCs.

40.-45. (canceled)

46. A composition comprising the dendritic cell subset-enriched itDCs produced by the method of claim 1.

47.-48. (canceled)

49. A dosage form comprising the composition of claim 39.

50. A process for producing a composition comprising dendritic cell subset-enriched itDCs comprising the steps of:
isolating cells of a dendritic cell subset that possess a desired physiological characteristic, and
generating induced tolerogenic dendritic cells (itDCs) from the cells of the dendritic cell subset.

51. (canceled)

52. Subset-enriched itDCs or a dosage form comprising subset-enriched itDCs obtainable by the method or process of claim 1.

53.-59. (canceled)
